(19) United States
(12) Patent Application Publication
(10) Pub. No.: US 2018/0312496 A1
(43) Pub. Date: Nov. 1, 2018

(54) BICYCLIC HETEROCYCLE DERIVATIVES AS BROMODOMAIN INHIBITORS

(71) Applicant: ORION CORPORATION, Espoo (FI)

(72) Inventors: Susanta SAMAJDAR, Bangalore (IN); Chandrasekhar ABBINENI, Hyderabad (IN); Tero LINNANEN, Tuusula (FI); Gerd WOHLFAHRT, Helsinki (FI)

(21) Appl. No.: 15/740,442

(22) PCT Filed: Jun. 30, 2016
(86) PCT No.: PCT/FI2016/050486

§ 371 Date: Dec. 28, 2017

(30) Foreign Application Priority Data
Jul. 2, 2015 (IN) 722/KOL/2015

(51) Int. Cl. C07D 413/14; C07D 417/14; C07D 417/04

(52) U.S. Cl. CPC C07D 413/14; C07D 417/04; C07D 417/14

(57) ABSTRACT

The invention relates to novel bicyclic heterocycle derivatives of formula (I)

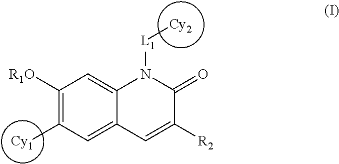

wherein Cy₁, Cy₂, R₁, R₂, and L₁ have the meaning given in the specification, and pharmaceutically acceptable salts thereof. The compounds of formula (I) are useful as bromodomain inhibitors in the treatment or prevention of diseases or disorders where bromodomain inhibition is desired.

### TECHNICAL FIELD

The present invention relates to novel bicyclic heterocycle derivatives of formula (I) which are useful as bromodomain inhibitors and to pharmaceutical compositions thereof.

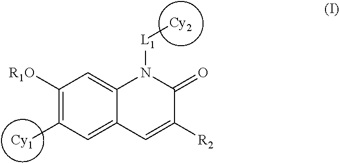

The invention relates also to the use of compounds of formula (I) for the treatment or prevention of diseases or disorders, in particular those where bromodomain inhibition is desired.

### BACKGROUND OF THE INVENTION

The acetylation of histone lysine is central for providing the dynamic regulation of chromatin-based gene transcription. The bromodomain (BRD), which is the conserved structural module in chromatin-associated proteins and histone acetyltranferases, is the sole protein domain known to recognize acetyl-lysine residues on proteins.

The BET family of bromodomain containing proteins comprises 4 proteins (BRD2, BRD3, BRD4 and BRD-t) which contain tandem bromodomains capable of binding to two acetylated lysine residues in close proximity, increasing the specificity of the interaction. BRD2 and BRD3 are reported to associate with histones along actively transcribed genes and may be involved in facilitating transcriptional elongation (Leroy et al., Mol. Cell., 2008, 30(1):51-60), while BRD4 appears to be involved in the recruitment of the pTEF-[beta] complex to inducible genes, resulting in phosphorylation of RNA polymerase and increased transcriptional output (Hargreaves et al., Cell, 2009, 138(1): 129-145). It has also been reported that BRD4 or BRD3 may fuse with NUT (nuclear protein in testis) forming novel fusion oncogenes, BRD4-NUT or BRD3-NUT, in a highly malignant form of epithelial neoplasia (French et al., Cancer Research, 2003, 63, 304-307 and French et al., Journal of Clinical Oncology, 2004, 22 (20), 4135-4139). Data suggests that BRD-NUT fusion proteins contribute to carcinogenesis (Oncogene, 2008, 27, 2237-2242). BRD-t is uniquely expressed in the testes and ovary. All family members have been reported to have some function in controlling or executing aspects of the cell cycle, and have been shown to remain in complex with chromosomes during cell division suggesting a role in the maintenance of epigenetic memory. In addition some viruses make use of these proteins to tether their genomes to the host cell chromatin, as part of the process of viral replication (You et al., Cell, 2004 1 17(3):349-60).

Japanese patent application JP 2008-156311 discloses a benzimidazole derivative which is said to be a BRD2 bromodomain binding agent which has utility with respect to virus infection/proliferation.

International patent application WO 2009/084693 discloses a series of thieno-triazolodiazepiene derivatives that are said to inhibit the binding between an acetylated histone and a bromodomain containing protein and are said to be useful as anti-cancer agents International patent application WO 2011/054846 discloses a series of quinoline derivatives that inhibit the binding of BET family bromodomains with acetylated lysine residues.

However, there remains a need for potent bromodomain inhibitors with desirable pharmaceutical properties. Certain bicyclic heterocycle derivatives have been found according to the present invention which inhibit the binding of BET family bromodomains to acetylated lysine residues. Such compounds will hereafter be referred to as “bromodomain inhibitors”.

### SUMMARY OF THE INVENTION

The present invention provides new bicyclic heterocycle derivatives which are able to inhibit the binding of BET family bromodomains to acetylated lysine residues. The present invention provides a compound of formula (I)

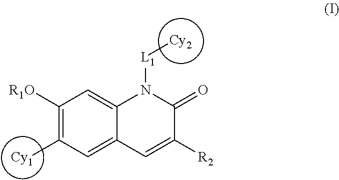

wherein

Cy₁ is an optionally substituted 5-6 membered monocyclic heterocyclyl ring containing 1-3 hetero atoms independently selected from N or O, which ring is optionally substituted by 1-3 C₁₋₇ alkyl groups;

Cy₂ is an optionally substituted aryl, optionally substituted C₃₋₁₀ cycloalkyl or optionally substituted 5-12 membered monocyclic or bicyclic heterocyclyl ring containing 1-3 hetero atoms independently selected from N, O or S; wherein the optional substitution at each occurrence is, independently, selected from 1-3 substituents selected from C₁₋₇ alkyl, C₁₋₇ alkoxy, halogen and —C(O)C₁₋₇ alkyl;

L₁ is —(CR₃R₃ₐ)ₙ;

R₁ is C₁₋₇ alkyl or halo C₁₋₇ alkyl;

R₂ is an optionally substituted aryl, optionally substituted aryl C₁₋₇ alkyl, optionally substituted heterocyclyl, optionally substituted heterocyclyl C₁₋₇ alkyl, —N(Rₐ)R_{b}, —(CH₂)ₘC(O)Rₐ₁, —(CH₂)ₘ₁C(O)ORₐ₂, —(CH₂)ₘ₂C(O)N(Rₐ₃)R_{b1}, —CH(CF₃)R_{d}, —S(O)₂N(Rₐ₄)R_{b2}, —(CRₐ₅R_{b3})ₘ₃C(O)ORₐ₆, —CH(CF₃)OR_{c}, —CH(CF₃)N(Rₐ₇)R_{b4}, or —ORₑ, wherein the optional substitution at each occurrence is, independently, selected from 1-3 substituents selected from C₁₋₇ alkyl, halo C₁₋₇ alkyl, —NHC(O)C₁₋₇ alkyl, amino, halogen, hydroxy, oxo, hydroxy C₁₋₇ alkyl, aryl, —N(H)C(O)C₁₋₇ alkyl, —(CH₂)ₘ₄C(O)OH or —(CH₂)ₘ₅C(O)NH(hydroxy C₁₋₇ alkyl);

Rₐ, Rₐ₁, Rₐ₂, Rₐ₃, Rₐ₄, Rₐ₅, Rₐ₆, Rₐ₇, R_{b}, R_{b1}, R_{b2}, R_{b3} and R_{b4} are independently selected from hydrogen, C₁₋₇ alkyl, hydroxy, C₁₋₇ alkoxy, hydroxy C₁₋₇ alkyl, halo C₁₋₇ alkyl, —S(O)₂C₁₋₇ alkyl, optionally substituted aryl, optionally substituted C₃₋₁₀ cycloalkyl, optionally substituted heterocyclyl or optionally substituted heterocyclyl C₁₋₇ alkyl; wherein the optional substitution at each occurrence is independently selected from 1-3 substituents selected from C₁₋₇ alkyl, halogen, hydroxy, hydroxy C₁₋₇ alkyl, C₁₋₇ alkoxy, cyano, halo C₁₋₇ alkyl and amino;

R_{c} is selected from C₁₋₇ alkyl or aryl wherein aryl is optionally substituted by 1-3 halogen atoms;

R_{d} is selected from optionally substituted heterocyclyl or optionally substituted aryl, wherein the optional substitution at each occurrence is independently selected from 1-3 substituents selected from C₁₋₇ alkyl and halogen;

Rₑ is selected from optionally substituted C₃₋₇ cycloalkyl or optionally substituted heterocyclyl, wherein the optional substitution at each occurrence is independently selected from 1-3 substituents selected from C₁₋₇ alkyl and halogen;

R₃ and R₃ₐ independently are selected from hydrogen, C₁₋₇ alkyl, hydroxy and halogen, or alternatively R₃ and R₃ₐ together with the carbon atom to which they are attached form a carbonyl (C═O) group;
m, m₁, m₂, m₃, m₄ and m₅ are, independently, an integer selected from 0, 1 or 2; and

n is an integer selected from 1, 2 or 3;

or a pharmaceutically acceptable salt thereof.

In a further aspect, the present invention provides a pharmaceutical composition comprising a compound of formula (I) or a pharmaceutically acceptable salt thereof.

In yet further aspect of the present invention, it provides a compound of formula (I) or a pharmaceutically acceptable thereof for use in the treatment or prevention of diseases or disorders where bromodomain inhibition is desired, in particular for the treatment or prevention of an autoimmune disease, inflammatory disease or cancer.

### DETAILED DESCRIPTION OF THE INVENTION

An embodiment of the present application provides compounds of formula (I) or pharmaceutically acceptable salts thereof which are useful as bromodomain inhibitors.

One of the embodiments of the present invention provides a compound of formula (I)

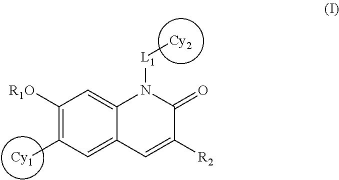

Cy₁ is an optionally substituted 5-6 membered monocyclic heterocyclyl ring containing 1-3 hetero atoms independently selected from N or O, which ring is optionally substituted by 1-3 C₁₋₇ alkyl groups;

Cy₂ is an optionally substituted aryl, optionally substituted C₃₋₁₀ cycloalkyl or optionally substituted 5-12 membered monocyclic or bicyclic heterocyclyl ring containing 1-3 hetero atoms independently selected from N, O or S; wherein the optional substitution at each occurrence is, independently, selected from 1-3 substituents selected from C₁₋₇ alkyl, C₁₋₇ alkoxy, halogen and —C(O)C₁₋₇ alkyl;

L₁ is —(CR₃R₃ₐ)ₙ;

R₁ is C₁₋₇ alkyl or halo C₁₋₇ alkyl;

R₂ is an optionally substituted aryl, optionally substituted aryl C₁₋₇ alkyl, optionally substituted heterocyclyl, optionally substituted heterocyclyl C₁₋₇ alkyl, —N(Rₐ)R_{b}, —(CH₂)ₘC(O)Rₐ₁, —(CH₂)ₘ₁C(O)ORₐ₂, —(CH₂)ₘ₂C(O)N(Rₐ₃)R_{b1}, —CH(CF₃)R_{d}, —S(O)₂N(Rₐ₄)R_{b2}, —(CRₐ₅R_{b3})ₘ₃C(O)ORₐ₆, —CH(CF₃)OR_{c}, —CH(CF₃)N(Rₐ₇)R_{b4}, or —ORₑ, wherein the optional substitution at each occurrence is, independently, selected from 1-3 substituents selected from C₁₋₇ alkyl, halo C₁₋₇ alkyl, —NHC(O)C₁₋₇ alkyl, amino, halogen, hydroxy, oxo, hydroxy C₁₋₇ alkyl, aryl, —N(H)C(O)C₁₋₇ alkyl, —(CH₂)ₘ₄C(O)OH or —(CH₂)ₘ₅C(O)NH(hydroxy C₁₋₇ alkyl); Rₐ, Rₐ₁, Rₐ₂, Rₐ₃, Rₐ₄, Rₐ₅, Rₐ₆, Rₐ₇, R_{b}, R_{b1}, R_{b2}, R_{b3} and R_{b4} are independently selected from hydrogen, C₁₋₇ alkyl, hydroxy, C₁₋₇ alkoxy, hydroxy C₁₋₇ alkyl, halo C₁₋₇ alkyl, —S(O)₂C₁₋₇ alkyl, optionally substituted aryl, optionally substituted C₃₋₁₀ cycloalkyl, optionally substituted heterocyclyl or optionally substituted heterocyclyl C₁₋₇ alkyl; wherein the optional substitution at each occurrence is independently selected from 1-3 substituents selected from C₁₋₇ alkyl, halogen, hydroxy, hydroxy C₁₋₇ alkyl, C₁₋₇ alkoxy, cyano, halo C₁₋₇ alkyl and amino;

R_{c} is selected from C₁₋₇ alkyl or aryl wherein aryl is optionally substituted by 1-3 halogen atoms;

R_{d} is selected from optionally substituted heterocyclyl or optionally substituted aryl, wherein the optional substitution at each occurrence is independently selected from 1-3 substituents selected from C₁₋₇ alkyl and halogen;

Rₑ is selected from optionally substituted C₃₋₇ cycloalkyl or optionally substituted heterocyclyl, wherein the optional substitution at each occurrence is independently selected from 1-3 substituents selected from C₁₋₇ alkyl and halogen;

R₃ and R₃ₐ independently are selected from hydrogen, C₁₋₇ alkyl, hydroxy and halogen, or alternatively R₃ and R₃ₐ together with the carbon atom to which they are attached form a carbonyl (C═O) group;

m, m₁, m₂, m₃, m₄ and m₅ are, independently, an integer selected from 0, 1 or 2; and

n is an integer selected from 1, 2 or 3;

or a pharmaceutically acceptable salt thereof.

It is to be understood that in case n is 2 or 3, each R₃ and R₃ₐ substituent in the L₁ chain can be selected independently of each other.

The embodiments below are illustrative of the present invention and are not intended to limit the claims to the specific embodiments exemplified.

According to one embodiment, specifically provided are compounds of formula (I), in which Cy₁ is 3,5-dimethylisoxazole.

According to another embodiment, specifically provided are compounds of formula (I), or according to any other embodiment or subclass referred to above, wherein R₁ is C₁₋₇ alkyl. As a subclass of this embodiment are compounds wherein R₁ is methyl.

According to another embodiment, specifically provided are compounds of formula (I), or according to any other embodiment or subclass referred to above, in which Cy₂ is a 5-12 membered monocyclic or bicyclic ring containing 0-2 hetero atoms independently selected from N and O, which ring is optionally substituted by 1-3 substituents selected from C₁₋₇ alkyl, C₁₋₇ alkoxy, halogen and —C(O)C₁₋₇ alkyl.

In a subclass of the above embodiment are compounds of formula (I), wherein Cy₂ is selected from optionally substituted pyridyl, optionally substituted phenyl, cyclohexyl, morpholinyl, optionally substituted piperazinyl or optionally substituted chromanyl; wherein the optional substitution at each occurrence is independently selected from 1-3 substituents selected from C₁₋₇ alkyl, C₁₋₇ alkoxy, halogen and —C(O)C₁₋₇ alkyl.

According to another embodiment, specifically provided are compounds of formula (I), or according to any other embodiment or subclass referred to above, wherein Cy₂ is optionally substituted by 1-2 substituents selected from C₁₋₇ alkoxy and halogen.

According to another embodiment, specifically provided are compounds of formula (I), or according to any other embodiment or subclass referred to above, wherein Cy₂ is selected from optionally substituted pyridyl or optionally substituted phenyl, wherein the optional substitution at each occurrence is independently selected from 1-2 substituents selected from C₁₋₇ alkoxy and halogen.

According to another embodiment, specifically provided are compounds of formula (I), or according to any other embodiment or subclass referred to above, wherein L₁ is —CH₂—, —(CH₂)₂—, —CH₂CH(OH)—, —CH₂CH(CH₃)— or —CH₂C(O)—, wherein the left bond is attached to the quinolin-2(1H)-one ring of formula (I).

According to another embodiment, specifically provided are compounds of formula (I), or according to any other embodiment or subclass referred to above, wherein R₂ is an optionally substituted 5-12 membered monocyclic or bicyclic ring containing 0-4 hetero atoms independently selected from N and O, which ring is optionally substituted by 1-3 substituents selected from C₁₋₇ alkyl, halogen, amino, hydroxy, —NHC(O)C₁₋₇ alkyl, halo C₁₋₇ alkyl, phenyl, oxo, hydroxy C₁₋₇ alkyl, —(CH₂)ₘ₅C(O)NH(hydroxy C₁₋₇ alkyl) or —(CH₂)ₘ₄C(O)OH.

In a subclass of the above embodiment are compounds of formula (I), wherein R₂ is phenyl, isoxazolyl, pyridinyl, pyrazolyl, imidazolyl, morpholinyl, 3,4-dihydroisoquinolinyl, 1,2,3,4-tetrahydroisoquinolinyl, 2-oxoimidazolidinyl, piperidinyl, pyrrolidinyl, indolinyl, 1,2,4-oxadiazol-5-yl or 1H-benzo[d]imidazole or azetidinyl; and the optional substituents are selected from 1-3 substituents selected from C₁₋₇ alkyl, halogen, amino, hydroxy, NHC(O)C₁₋₇ alkyl, halo C₁₋₇ alkyl, phenyl, oxo, hydroxy C₁₋₇ alkyl, —(CH₂)ₘ₅C(O)NH(hydroxy C₁₋₇ alkyl) or —(CH₂)ₘ₄C(O)OH.

According to yet another embodiment, specifically provided are compounds of formula (I), or according to any other embodiment or subclass referred to above, in which R₂ is —(CH₂)ₘC(O)Rₐ₁, in particular wherein Rₐ₁ is a 5-12 membered monocyclic or bicyclic ring containing 0-4 hetero atoms independently selected from N and O, which ring is optionally substituted by one hydroxy or halogen group, and m is 0 or 1. In a subclass of this embodiment are compounds wherein Rₐ₁ is phenyl, piperidinyl, pyrrolidinyl, azetidinyl or indolinyl which rings are optionally substituted by one hydroxy or halogen group, and m is 0 or 1.

According to yet another embodiment, specifically provided are compounds of formula (I), or according to any other embodiment or subclass referred to above, wherein R₂ is —(CH₂)ₘ₂C(O)N(Rₐ₃)R_{b1}; in particular wherein Rₐ₃ is hydrogen or C₁₋₇ alkyl, and R_{b1} is hydrogen, C₁₋₇ alkyl, hydroxy C₁₋₇ alkyl, halo C₁₋₇ alkyl, optionally substituted C₃₋₁₀ cycloalkyl, optionally substituted heterocyclyl, optionally substituted phenyl or optionally substituted heterocyclyl C₁₋₇ alkyl, wherein heterocyclyl at each occurrence means a 5-12 membered monocyclic or bicyclic ring containing 1-4 hetero atoms independently selected from N, O and S, and wherein the optional substitution at each occurrence is, independently, selected from 1-3 substituents selected from C₁₋₇ alkyl, hydroxy, halogen, halo C₁₋₇ alkyl, amino, cyano, C₁₋₇ alkoxy or oxo; and m2 is 0 or 1.

In a subclass of the above embodiment are compounds of formula (I), wherein R_{b1} is cyclohexyl, pyridinyl, piperidinyl, 1,3,4-thiadiazolyl, pyrazolyl, phenyl or imidazolyl C₁₋₇ alkyl, which groups are optionally substituted by 1-3 substituents independently selected from C₁₋₇ alkyl, hydroxy, halogen, halo C₁₋₇ alkyl, amino, cyano, C₁₋₇ alkoxy or oxo.

According to yet another embodiment, specifically provided are compounds of formula (I), or according to any other embodiment or subclass referred to above, wherein Rₐ₃ is hydrogen.

According to yet another embodiment, specifically provided are compounds of formula (I), or according to any other embodiment or subclass referred to above, wherein R₂ is —N(Rₐ)R_{b}; in particular wherein Rₐ is hydrogen and R_{b} is hydrogen, hydroxy C₁₋₇ alkyl, —SO₂-methyl or optionally substituted 5-12 membered monocyclic or bicyclic ring containing 1-4 hetero atoms independently selected from N, O and S, and wherein the optional substitution is selected from 1-3 substituents selected from C₁₋₇ alkyl, hydroxy, halogen, halo C₁₋₇ alkyl or C₁₋₇ alkoxy.

According to yet another embodiment, specifically provided are compounds of formula (I), or according to any other embodiment or subclass referred to above, wherein R₂ is —CH(CF₃)R_{d}, —CH(CF₃)OR or —CH(CF₃)N(Rₐ₇)R_{b4}; in particular those wherein R_{d} is morpholinyl, R of is 4-fluorophenyl or C₁₋₇ alkyl, Rₐ₇ is hydrogen and R_{b4} is hydroxy C₁₋₇ alkyl or 4-fluorophenyl.

According to yet another embodiment, specifically provided are compounds of formula (I), or according to any other embodiment or subclass referred to above, wherein n is 1 or 2.

According to yet another embodiment of the present invention, the compound of formula (I) is a compound of formula (IA):

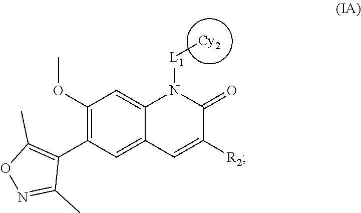

wherein R₂, L₁ and Cy₂ are same as defined in formula (I), or a pharmaceutically acceptable salt thereof.

According to yet another embodiment of the present invention, the compound of formula (I) is a compound of formula (IB):

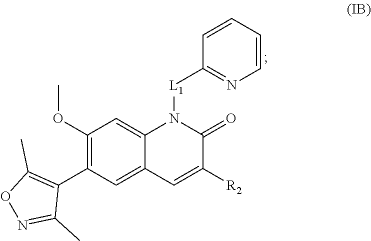

wherein, R₂ and L₁ are same as defined in formula (I), or a pharmaceutically acceptable salt thereof. In a subgroup of this embodiment are compounds wherein L₁ is —CH₂—.

According to yet another embodiment of the present invention, the compound of formula (I) is a compound of formula (IC):

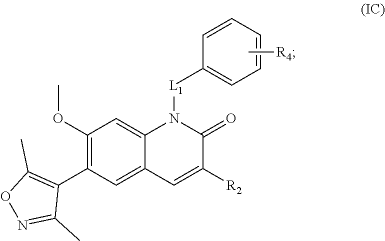

wherein R₂ and L₁ are same as defined in formula (I), and R₄ is hydrogen, C₁₋₇ alkoxy, or halogen, or a pharmaceutically acceptable salt thereof.

According to yet another embodiment, specifically provided are compounds of formula (I), or according to any other embodiment or subclass referred to above, wherein the heterocyclyl group, at each occurrence, independently, is a 5-12 membered monocyclic or bicyclic ring containing 1-4 hetero atoms independently selected from N, O and S.

According to yet another embodiment, specifically provided are compounds of formula (I), or according to any other embodiment or subclass referred to above, wherein Rₑ is cyclopropyl or tetrahydro-2H-pyran-4-yl.

In yet another particular embodiment of the present invention, the compound of formula (I) is selected from the group consisting of:

| | |
|---|---|
| 1. | 3,6-Bis(3,5-dimethylisoxazol-4-yl)-7-methoxy-1-(pyridin-2- |
| | ylmethyl)quinolin-2(1H)-one; |
| 2. | 6-(3,5-Dimethylisoxazol-4-yl)-7-methoxy-1-(pyridin-2-ylmethyl)- |
| | 3-(pyridin-4-yl)quinolin-2(1H)-one; |
| 3. | 6-(3,5-Dimethylisoxazol-4-yl)-7-methoxy-3-(naphthalen-2-yl)-1- |
| | (pyridin-2-ylmethyl)quinolin-2(1H)-one; |
| 4. | N-(3-(6-(3,5-dimethylisoxazol-4-yl)-7-methoxy-2-oxo-1-(pyridin- |
| | 2-yl-methyl)-1,2-dihydroquinolin-3-yl)phenyl)acetamide; |
| 5. | 6-(3,5-Dimethylisoxazol-4-yl)-7-methoxy-3-phenyl-1-(pyridin-2- |
| | ylmethyl)-quinolin-2(1H)-one; |
| 6. | 3-(2,4-Difluorophenyl)-6-(3,5-dimethylisoxazol-4-yl)-7-methoxy- |
| | 1-(pyridin-2-ylmethyl)quinolin-2(1H)-one; |
| 7. | 6-(3,5-Dimethylisoxazol-4-yl)-3-(4-hydroxyphenyl)-7-methoxy-1- |
| | (pyridin-2-ylmethyl)quinolin-2(1H)-one; |
| 8. | 6-(3,5-Dimethylisoxazol-4-yl)-7-methoxy-1-(pyridin-2-ylmethyl)- |
| | 3-(2-(tri-fluoromethyl)phenyl)quinolin-2(1H)-one; |
| 9. | 6-(3,5-Dimethylisoxazol-4-yl)-7-methoxy-3-(1-methyl-1H- |
| | pyrazol-4-yl)-1-(pyridin-2-ylmethyl)quinolin-2(1H)-one; |
| 10. | 6-(3,5-Dimethylisoxazol-4-yl)-7-methoxy-1-(pyridin-2-ylmethyl)- |
| | 3-(pyridin-3-yl)quinolin-2(1H)-one; |
| 11. | 6-(3,5-Dimethylisoxazol-4-yl)-3-(5-(hydroxymethyl)-3-methyl- |
| | isoxazol-4-yl)-7-methoxy-1-(pyridin-2-ylmethyl)quinolin-2(1H)- |
| | one; |
| 12. | 6-(3,5-Dimethylisoxazol-4-yl)-7-methoxy-3-(1H-pyrazol-4-yl)-1- |
| | (pyridin-2-ylmethyl)quinolin-2(1H)-one; |
| 13. | 1-(4-Chlorophenethyl)-6-(3,5-dimethylisoxazol-4-yl)-3-(4- |
| | hydroxyphenyl)-7-methoxyquinolin-2(1H)-one; |
| 14. | 1-(4-Chlorophenethyl)-6-(3,5-dimethylisoxazol-4-yl)-7-methoxy- |
| | 3-(1-methyl-1H-pyrazol-4-yl)quinolin-2(1H)-one; |
| 15. | 6-(3,5-Dimethylisoxazol-4-yl)-3-(1H-imidazol-1-yl)-7-methoxy- |
| | 1-(pyridin-2-ylmethyl)quinolin-2(1H)-one; |
| 16. | 6-(3,5-Dimethylisoxazol-4-yl)-7-methoxy-3-(1H-pyrazol-1-yl)-1- |
| | (pyridin-2-ylmethyl)quinolin-2(1H)-one; |
| 17. | 6-(3,5-Dimethylisoxazol-4-yl)-7-methoxy-3-(4-phenyl-1H- |
| | imidazol-1-yl)-1-(pyridin-2-ylmethyl)quinolin-2(1H)-one; |
| 18. | 6-(3,5-Dimethylisoxazol-4-yl)-7-methoxy-3-morpholino-1- |
| | (pyridin-2-yl-methyl)quinolin-2(1H)-one; |
| 19. | 6-(3,5-Dimethylisoxazol-4-yl)-3-((2-hydroxyethyl)amino)-7- |
| | methoxy-1-(pyridin-2-ylmethyl)quinolin-2(1H)-one; |
| 20. | N-(6-(3,5-dimethylisoxazol-4-yl)-7-methoxy-2-oxo-1-(pyridin-2- |
| | ylmethyl)-1,2-dihydroquinolin-3-yl)methanesulfonamide; |
| 21. | 3-(3,4-Dihydroisoquinolin-2(1H)-yl)-6-(3,5-dimethylisoxazol-4- |
| | yl)-7-methoxy-1-(pyridin-2-ylmethyl)quinolin-2(1H)-one; |
| 22. | 6-(3,5-Dimethylisoxazol-4-yl)-7-methoxy-3-((1-methylpiperidin- |
| | 4-yl)amino)-1-(pyridin-2-ylmethyl)quinolin-2(1H)-one; |
| 23. | 6-(3,5-Dimethylisoxazol-4-yl)-7-methoxy-3-(2-oxoimidazolidin- |
| | 1-yl)-1-(pyridin-2-ylmethyl)quinolin-2(1H)-one; |
| 24. | 1-(4-Chlorophenethyl)-6-(3,5-dimethylisoxazol-4-yl)-7-methoxy- |
| | 3-(2-oxo-imidazolidin-1-yl)quinolin-2(1H)-one; |
| 25. | 6-(3,5-Dimethylisoxazol-4-yl)-3-(4-hydroxypiperidine-1- |
| | carbonyl)-7-methoxy-1-(pyridin-2-ylmethyl)quinolin-2(1H)-one; |
| 26. | 6-(3,5-Dimethylisoxazol-4-yl)-3-(3-hydroxypyrrolidine-1- |
| | carbonyl)-7-methoxy-1-(pyridin-2-ylmethyl)quinolin-2(1H)-one; |
| 27. | 6-(3,5-Dimethylisoxazol-4-yl)-N-((1r,4r-4-hydroxycyclohexyl)- |
| | 7-methoxy-2-oxo-1-(pyridin-2-ylmethyl)-1,2-dihydroquinoline- |
| | 3-carboxamide; |
| 28. | 6-(3,5-Dimethylisoxazol-4-yl)-N-(5-(hydroxymethyl)-1,3,4- |
| | thiadiazol-2-yl)-7-methoxy-2-oxo-1-(pyridin-2-ylmethyl)-1,2- |
| | dihydroquinoline-3-carboxamide; |
| 29. | 6-(3,5-Dimethylisoxazol-4-yl)-N-ethyl-7-methoxy-2-oxo-1- |
| | (pyridin-2-yl-methyl)-1,2-dihydroquinoline-3-carboxamide; |
| 30. | 6-(3,5-Dimethylisoxazol-4-yl)-N-(3-hydroxypropyl)-7-methoxy- |
| | 2-oxo-1-(pyridin-2-ylmethyl)-1,2-dihydroquinoline-3- |
| | carboxamide; |
| 31. | 6-(3,5-Dimethylisoxazol-4-yl)-7-methoxy-N-methyl-2-oxo-1- |
| | (pyridin-2-yl-methyl)-1,2-dihydroquinoline-3-carboxamide; |
| 32. | 6-(3,5-Dimethylisoxazol-4-yl)-7-methoxy-2-oxo-1-(pyridin-2- |
| | ylmethyl)-N-(5-(trifluoromethyl)-1,3,4-thiadiazol-2-yl)-1,2- |
| | dihydroquinoline-3-carboxamide; |
| 33. | 6-(3,5-Dimethylisoxazol-4-yl)-N-(4-fluorophenyl)-7-methoxy-2- |
| | oxo-1-(pyridin-2-ylmethyl)-1,2-dihydroquinoline-3-carboxamide; |
| 34. | 6-(3,5-Dimethylisoxazol-4-yl)-7-methoxy-2-oxo-1-(pyridin-2- |
| | ylmethyl)-N-(4-(trifluoromethyl)phenyl)-1,2-dihydroquinoline-3- |
| | carboxamide; |
| 35. | 6-(3,5-Dimethylisoxazol-4-yl)-7-methoxy-N-(4-methoxyphenyl)- |
| | 2-oxo-1-(pyridin-2-ylmethyl)-1,2-dihydroquinoline-3- |
| | carboxamide; |
| 36. | 6-(3,5-Dimethylisoxazol-4-yl)-7-methoxy-2-oxo-1-(pyridin-2- |
| | ylmethyl)-N-(2,2,2-trifluoroethyl)-1,2-dihydroquinoline-3- |
| | carboxamide; |
| 37. | 3-(Azetidine-1-carbonyl)-6-(3,5-dimethylisoxazol-4-yl)-7- |
| | methoxy-1-(pyridin-2-ylmethyl)quinolin-2(1H)-one; |
| 38. | 6-(3,5-Dimethylisoxazol-4-yl)-7-methoxy-2-oxo-1-(pyridin-2- |
| | ylmethyl)-N-(pyridin-4-yl)-1,2-dihydroquinoline-3-carboxamide; |
| 39. | N-(3-(1H-imidazol-1-yl)propyl)-6-(3,5-dimethylisoxazol-4-yl)-7- |
| | methoxy-2-oxo-1-(pyridin-2-ylmethyl)-1,2-dihydroquinoline-3- |
| | carboxamide; |
| 40. | 6-(3,5-Dimethylisoxazol-4-yl)-N-(4-hydroxyphenyl)-7-methoxy- |
| | 2-oxo-1-(pyridin-2-ylmethyl)-1,2-dihydroquinoline-3- |
| | carboxamide; |
| 41. | 6-(3,5-Dimethylisoxazol-4-yl)-7-methoxy-2-oxo-N-(pyridin-2- |
| | yl)-1-(pyridin-2-ylmethyl)-1,2-dihydroquinoline-3-carboxamide; |
| 42. | 6-(3,5-Dimethylisoxazol-4-yl)-7-methoxy-2-oxo-1-(pyridin-2- |
| | ylmethyl)-N-(pyridin-3-yl)-1,2-dihydroquinoline-3-carboxamide; |
| 43. | 6-(3,5-Dimethylisoxazol-4-yl)-7-methoxy-N-(1-methyl-1H- |
| | pyrazol-3-yl)-2-oxo-1-(pyridin-2-ylmethyl)-1,2-dihydroquinoline- |
| | 3-carboxamide; |
| 44. | N-(4-Chlorophenyl)-6-(3,5-dimethylisoxazol-4-yl)-7-methoxy-2- |
| | oxo-1-(pyridin-2-ylmethyl)-1,2-dihydroquinoline-3-carboxamide; |
| 45. | N-(4-Cyanophenyl)-6-(3,5-dimethylisoxazol-4-yl)-7-methoxy-2- |
| | oxo-1-(pyridin-2-ylmethyl)-1,2-dihydroquinoline-3-carboxamide; |
| 46. | 6-(3,5-Dimethylisoxazol-4-yl)-3-(5-hydroxyindoline-1-carbonyl)- |
| | 7-methoxy-1-(pyridin-2-ylmethyl)quinolin-2(1H)-one; |
| 47. | 6-(3,5-Dimethylisoxazol-4-yl)-7-methoxy-N-(1-methylpiperidin- |
| | 4-yl)-2-oxo-1-(pyridin-2-ylmethyl)-1,2-dihydroquinoline-3- |
| | carboxamide; |
| 48. | 6-(3,5-Dimethylisoxazol-4-yl)-N-(4-hydroxy-3-methylphenyl)-7- |
| | methoxy-2-oxo-1-(pyridin-2-ylmethyl)-1,2-dihydroquinoline-3- |
| | carboxamide; |
| 49. | 6-(3,5-Dimethylisoxazol-4-yl)-3-(3-hydroxyazetidine-1-carbonyl)- |
| | 7-methoxy-1-(pyridin-2-ylmethyl)quinolin-2(1H)-one; |
| 50. | 6-(3,5-Dimethylisoxazol-4-yl)-N-(6-hydroxypyridin-3-yl)-7- |
| | methoxy-2-oxo-1-(pyridin-2-ylmethyl)-1,2-dihydroquinoline-3- |
| | carboxamide; |
| 51. | 6-(3,5-Dimethylisoxazol-4-yl)-N-(3-fluoro-4-hydroxyphenyl)-7- |
| | methoxy-2-oxo-1-(pyridin-2-ylmethyl)-1,2-dihydroquinoline-3- |
| | carboxamide; |
| 52. | 2-(6-(3,5-Dimethylisoxazol-4-yl)-7-methoxy-2-oxo-1-(pyridin-2- |
| | ylmethyl)-1,2-dihydroquinolin-3-yl)-N-(4-hydroxyphenyl) |
| | acetamide; |
| 53. | 6-(3,5-Dimethylisoxazol-4-yl)-N-(3-hydroxyphenyl)-7-methoxy- |
| | 2-oxo-1-(pyridin-2-ylmethyl)-1,2-dihydroquinoline-3- |
| | carboxamide; |
| 54. | 6-(3,5-Dimethylisoxazol-4-yl)-N-(4-hydroxyphenyl)-7-methoxy- |
| | N-methyl-2-oxo-1-(pyridin-2-ylmethyl)-1,2-dihydroquinoline-3- |
| | carboxamide; |
| 55. | 6-(3,5-Dimethylisoxazol-4-yl)-N-(4-hydroxy-3,5-dimethylphenyl)- |
| | 7-methoxy-2-oxo-1-(pyridin-2-ylmethyl)-1,2-dihydroquinoline-3- |
| | carboxamide; |
| 56. | 2-(6-(3,5-Dimethylisoxazol-4-yl)-7-methoxy-2-oxo-1-(pyridin-2- |
| | ylmethyl)-1,2-dihydroquinolin-3-yl)-N-ethylacetamide; |
| 57. | 3-(2-(Azetidin-1-yl)-2-oxoethyl)-6-(3,5-dimethylisoxazol-4-yl)-7- |
| | methoxy-1-(pyridin-2-ylmethyl)quinolin-2(1H)-one; |
| 58. | 2-(6-(3,5-Dimethylisoxazol-4-yl)-7-methoxy-2-oxo-1-(pyridin-2- |
| | ylmethyl)-1,2-dihydroquinolin-3-yl)acetamide; |
| 59. | 2-(6-(3,5-Dimethylisoxazol-4-yl)-7-methoxy-2-oxo-1-(pyridin-2- |
| | ylmethyl)-1,2-dihydroquinolin-3-yl)-N-(4-hydroxy-3,5- |
| | dimethylphenyl)acetamide; |
| 60. | 6-(3,5-Dimethylisoxazol-4-yl)-N-(2,6-dimethylpyridin-4-yl)-7- |
| | methoxy-2-oxo-1-(pyridin-2-ylmethyl)-1,2-dihydroquinoline-3- |
| | carboxamide; |
| 61. | 6-(3,5-Dimethylisoxazol-4-yl)-N,N-diethyl-7-methoxy-2-oxo-1- |
| | (pyridin-2-ylmethyl)-1,2-dihydroquinoline-3-carboxamide; |
| 62. | 6-(3,5-Dimethylisoxazol-4-yl)-N-(4,6-dimethylpyridin-2-yl)-7- |
| | methoxy-2-oxo-1-(pyridin-2-ylmethyl)-1,2-dihydroquinoline-3- |
| | carboxamide; |
| 63. | N-(6-aminopyridin-3-yl)-6-(3,5-dimethylisoxazol-4-yl)-7- |
| | methoxy-2-oxo-1-(pyridin-2-ylmethyl)-1,2-dihydroquinoline-3- |
| | carboxamide; |
| 64. | 1-(2,4-Difluorobenzyl)-6-(3,5-dimethylisoxazol-4-yl)-N-(2,6- |
| | dimethylpyridin-4-yl)-7-methoxy-2-oxo-1,2-dihydroquinoline-3- |
| | carboxamide; |
| 65. | 6-(3,5-Dimethylisoxazol-4-yl)-N-(2,6-dimethylpyridin-4-yl)-7- |
| | methoxy-1-((3-methoxypyridin-2-yl)methyl)-2-oxo-1,2- |
| | dihydroquinoline-3-carboxamide |
| 66. | 6-(3,5-Dimethylisoxazol-4-yl)-3-(1H-imidazol-2-yl)-7-methoxy- |
| | 1-(pyridin-2-ylmethyl)quinolin-2(1H)-one; |
| 67. | 6-(3,5-Dimethylisoxazol-4-yl)-7-methoxy-3-(1-phenyl-1H- |
| | imidazol-2-yl)-1-(pyridin-2-ylmethyl)quinolin-2(1H)-one; |
| 68. | 6-(3,5-Dimethylisoxazol-4-yl)-3-(hydroxydiphenylmethyl)-7- |
| | methoxy-1-(pyridin-2-ylmethyl)quinolin-2(1H)-one; |
| 69. | 6-(3,5-Dimethylisoxazol-4-yl)-7-methoxy-1-(pyridin-2-ylmethyl)- |
| | 3-(2,2,2-trifluoro-1-(4-fluorophenoxy)ethyl)quinolin-2(1H)-one; |
| 70. | 6-(3,5-Dimethylisoxazol-4-yl)-7-methoxy-1-(pyridin-2-ylmethyl)- |
| | 3-(2,2,2-trifluoro-1-morpholinoethyl)quinolin-2(1H)-one; |
| 71. | 6-(3,5-Dimethylisoxazol-4-yl)-7-methoxy-1-(pyridin-2-ylmethyl)- |
| | 3-(2,2,2-trifluoro-1-((2-hydroxyethyl)amino)ethyl)quinolin-2(1H)- |
| | one; |
| 72. | 7-Methoxy-6-(3-methylisoxazol-4-yl)-1-(pyridin-2-ylmethyl)-3- |
| | (2,2,2-trifluoro-1-((4-hydroxyphenyl)amino)ethyl)quinolin-2(1H)- |
| | one; |
| 73. | 6-(3,5-Dimethylisoxazol-4-yl)-3-(1-ethoxy-2,2,2-trifluoroethyl)- |
| | 7-methoxy-1-(pyridin-2-ylmethyl)quinolin-2(1H)-one; |
| 74. | 6-(3,5-Dimethylisoxazol-4-yl)-7-methoxy-1-(pyridin-2-ylmethyl)- |
| | 3-(2,2,2-trifluoro-1-isobutoxyethyl)quinolin-2(1H)-one; |
| 75. | 6-(3,5-Dimethylisoxazol-4-yl)-3-(3-ethyl-1,2,4-oxadiazol-5-yl)-7- |
| | methoxy-1-(pyridin-2-ylmethyl)quinolin-2(1H)-one; |
| 76. | 3-Benzoyl-6-(3,5-dimethylisoxazol-4-yl)-7-methoxy-1-(pyridin- |
| | 2-ylmethyl)-quinolin-2(1H)-one; |
| 77. | 2-(6-(3,5-Dimethylisoxazol-4-yl)-7-methoxy-2-oxo-1-(pyridin-2- |
| | ylmethyl)-1,2-dihydroquinolin-3-yl)acetic acid; |
| 78. | 2-(1-(4-Chlorophenethyl)-6-(3,5-dimethylisoxazol-4-yl)-7- |
| | methoxy-2-oxo-1,2-dihydroquinolin-3-yl)acetic acid; |
| 79. | 2-(6-(3,5-Dimethylisoxazol-4-yl)-7-methoxy-1-(4- |
| | methoxybenzyl)-2-oxo-1,2-dihydroquinolin-3-yl)acetic acid; |
| 80. | 2-(4-(6-(3,5-Dimethylisoxazol-4-yl)-7-methoxy-2-oxo-1-(pyridin- |
| | 2-yl-methyl)-1,2-dihydroquinolin-3-yl)-1H-pyrazol-1-yl)-N-(2- |
| | hydroxyethyl)-acetamide; |
| 81. | 2-(4-(6-(3,5-Dimethylisoxazol-4-yl)-7-methoxy-2-oxo-1-(pyridin- |
| | 2-yl-methyl)-1,2-dihydroquinolin-3-yl)-1H-pyrazol-1- |
| | yl)acetic acid; |
| 82. | 6-(3,5-Dimethylisoxazol-4-yl)-7-methoxy-2-oxo-N-(pyridin-2-yl)- |
| | 1-(pyridin-2-ylmethyl)-1,2-dihydroquinoline-3-sulfonamide; |
| 83. | 6-(3,5-Dimethylisoxazol-4-yl)-3-(6-hydroxy-1H-benzo[d] |
| | imidazol-2-yl)-7-methoxy-1-(pyridin-2-ylmethyl)quinolin-2(1H)- |
| | one; |
| 84. | 3-(Azetidine-1-carbonyl)-1-(4-chlorobenzyl)-6-(3,5- |
| | dimethylisoxazol-4-yl)-7-methoxyquinolin-2(1H)-one; |
| 85. | 3-(Azetidine-1-carbonyl)-6-(3,5-dimethylisoxazol-4-yl)-7- |
| | methoxy-1-(2-morpholinoethyl)quinolin-2(1H)-one; |
| 86. | 3-(Azetidine-1-carbonyl)-6-(3,5-dimethylisoxazol-4-yl)-7- |
| | methoxy-1-(2-(4-pivaloylpiperazin-1-yl)ethyl)quinolin-2(1H)- |
| | one; |
| 87. | 3-(Azetidine-1-carbonyl)-1-benzyl-6-(3,5-dimethylisoxazol-4-yl)- |
| | 7-methoxyquinolin-2(1H)-one; |
| 88. | 3-(Azetidine-1-carbonyl)-1-(4-chlorophenethyl)-6-(3,5-dimethyl- |
| | isoxazol-4-yl)-7-methoxyquinolin-2(1H)-one; |
| 89. | 3-(Azetidine-1-carbonyl)-1-(3,4-dichlorophenethyl)-6-(3,5- |
| | dimethylisoxazol-4-yl)-7-methoxyquinolin-2(1H)-one; |
| 90. | 3-(Azetidine-1-carbonyl)-6-(3,5-dimethylisoxazol-4-yl)-1-(4- |
| | fluorophen-ethyl)-7-methoxyquinolin-2(1H)-one; |
| 91. | 3-(Azetidine-1-carbonyl)-6-(3,5-dimethylisoxazol-4-yl)-7- |
| | methoxy-1-(2-(pyridin-3-yl)ethyl)quinolin-2(1H)-one; |
| 92. | 3-(Azetidine-1-carbonyl)-1-(2-(4-chlorophenyl)propyl)-6-(3,5- |
| | dimethyl-isoxazol-4-yl)-7-methoxyquinolin-2(1H)-one; |
| 93. | 3-(Azetidine-1-carbonyl)-1-(2-(4-chlorophenyl)-2-hydroxyethyl)- |
| | 6-(3,5-di-methylisoxazol-4-yl)-7-methoxyquinolin-2(1H)-one; |
| 94. | 3-(Azetidine-1-carbonyl)-1-(2-cyclohexylethyl)-6-(3,5- |
| | dimethylisoxazol-4-yl)-7-methoxyquinolin-2(1H)-one; |
| 95. | 3-(Azetidine-1-carbonyl)-1-(2-(2,2-dimethylchroman-6-yl)ethyl)- |
| | 6-(3,5-di-methylisoxazol-4-yl)-7-methoxyquinolin-2(1H)-one; |
| 96. | 3-(Azetidine-1-carbonyl)-6-(3,5-dimethylisoxazol-4-yl)-7- |
| | methoxy-1-phen-ethylquinolin-2(1H)-one; |
| 97. | 3-(Azetidine-1-carbonyl)-1-(3-chlorophenethyl)-6-(3,5- |
| | dimethylisoxazol-4-yl)-7-methoxyquinolin-2(1H)-one; |
| 98. | 6-(3,5-Dimethylisoxazol-4-yl)-N-(4-hydroxy-3,5-dimethylphenyl)- |
| | 7-methoxy-N-methyl-2-oxo-1-(pyridin-2-ylmethyl)-1,2- |
| | dihydroquinoline-3-carboxamide; |
| 99. | 6-(3,5-Dimethylisoxazol-4-yl-3-((5-hydroxydolin-1-yl)methyl-7- |
| | methoxy-1-(pyridin-2-ylmethyl)quinolin-2(1H)-one; |
| 100. | 3-((1H-tetrazol-5-yl)methyl)-6-(3,5-dimethylisoxazol-4-yl)-7- |
| | methoxy-1-(pyridin-2-ylmethyl)quinolin-2(1H)-one; |
| 101. | 2-(6-(3,5-Dimethylisoxazol-4-yl)-7-methoxy-2-oxo-1-(pyridin-2- |
| | ylmethyl)-1,2-dihydroquinolin-3-yl)-2-methylpropanoic acid; |
| 102. | 2-(1-(4-Chlorophenethyl)-6-(3,5-dimethylisoxazol-4-yl)-7- |
| | methoxy-2-oxo-1,2-dihydroquinolin-3-yl)-2-methylpropanoic |
| | acid; |
| 103. | 3-(azetidine-1-carbonyl)-1-(2-(4-chlorophenyl)-2-oxoethyl)-6- |
| | (3,5-dimethyl-isoxazol-4-yl)-7-methoxyquinolin-2(1H)-one; |
| 104. | 6-(3,5-Dimethylisoxazol-4-yl)-N-(4,6-dimethylpyridin-2-yl)-7- |
| | methoxy-2-oxo-1-(pyridin-2-ylmethyl)-1,2-dihydroquinoline-3- |
| | carboxamide hydrochloride; |
| 105. | 3-(6-Aminopyridin-3-yl)-6-(3,5-dimethylisoxazol-4-yl)-7- |
| | methoxy-1-(pyridin-2-ylmethyl)quinolin-2(1H)-one; |
| 106. | N-(6-Amino-5-methylpyridin-3-yl)-6-(3,5-dimethylisoxazol-4-yl)- |
| | 7-methoxy-2-oxo-1-(pyridin-2-ylmethyl)-1,2-dihydroquinoline-3- |
| | carboxamide; |
| 107. | 6-(3,5-Dimethylisoxazol-4-yl)-3-((2,6-dimethylpyridin-4-yl) |
| | amino)-7-methoxy-1-(pyridin-2-ylmethyl)quinolin-2(1H)-one; |
| 108. | 3-(6-Amino-5-methylpyridin-3-yl)-6-(3,5-dimethylisoxazol-4-yl)- |
| | 7-methoxy-1-(pyridin-2-ylmethyl)quinolin-2(1H)-one; |
| 109. | 3-(Azetidine-1-carbonyl)-6-(3,5-dimethylisoxazol-4-yl)-7- |
| | methoxy-1-((3-methoxypyridin-2-yl)methyl)quinolin-2(1H)-one; |
| 110. | 3-(6-Aminopyridin-3-yl)-6-(3,5-dimethylisoxazol-4-yl)-7- |
| | methoxy-1-((3-methoxypyridin-2-yl)methyl)quinolin-2(1H)-one; |
| 111. | 6-(3,5-Dimethylisoxazol-4-yl)-N-(4-hydroxy-3,5-dimethylphenyl)- |
| | 7-methoxy-1-((3-methoxypyridin-2-yl)methyl)-2-oxo-1,2- |
| | dihydroquinoline-3-carboxamide; |
| 112. | N-(6-amino-5-methylpyridin-3-yl)-6-(3,5-dimethylisoxazol-4-yl)- |
| | 7-methoxy-1-((3-methoxypyridin-2-yl)methyl)-2-oxo-1,2- |
| | dihydroquinoline-3-carboxamide |
| 113. | N-(4-amino-3,5-dimethylphenyl)-6-(3,5-dimethylisoxazol-4-yl)- |
| | 7-methoxy-2-oxo-1-(pyridin-2-ylmethyl)-1,2-dihydroquinoline- |
| | 3-carboxamide |
| 114. | N-(1,5-dimethyl-6-oxo-1,6-dihydropyridin-3-yl)-6-(3,5- |
| | dimethylisoxazol-4-yl)-7-methoxy-2-oxo-1-(pyridin-2-ylmethyl)- |
| | 1,2-dihydroquinoline-3-carboxamide |
| 115. | 6-(3,5-Dimethylisoxazol-4-yl)-3-(3-fluoropyrrolidine-1-carbonyl)- |
| | 7-methoxy-1-(pyridin-2-ylmethyl)quinolin-2(1H)-one |
| 116. | 6-(3,5-Dimethylisoxazol-4-yl)-7-methoxy-1-(pyridin-2-ylmethyl)- |
| | 3-((tetrahydro-2H-pyran-4-yl)oxy)quinolin-2(1H)-one |
| 117. | 3-Cyclopropoxy-6-(3,5-dimethylisoxazol-4-yl)-7-methoxy-1- |
| | (pyridin-2-yl-methyl)quinolin-2(1H)-one |

or a pharmaceutically acceptable salt or tautomer thereof.

In yet another embodiment according to the present patent application, it provides a pharmaceutical composition comprising a compound of formula (I), (IA), (IB) or (IC) and at least one pharmaceutically acceptable excipient (such as a pharmaceutically acceptable carrier or diluent). Preferably, the pharmaceutical composition comprises a therapeutically effective amount of at least one compound described herein.

It should be understood that the compounds of the invention including those according to formulas (I), (IA), (IB) or (IC) encompass all stereoisomers, enantiomers, diastereomers or geometrical isomers that may be contemplated from the chemical structure of the compounds.

The present compounds may also exist as tautomers or equilibrium mixtures thereof wherein a proton of a compound shifts from one atom to another. Examples of tautomers include, but are not limited to, amido-imido, keto-enol, phenol-keto, oxime-nitroso, nitro-aci, imine-enamine and the like. All tautomeric forms of the compounds are intended to be encompassed by their structural formula even though only one tautomeric form may be depicted.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “C₁₋₇ alkyl”, as employed herein as such or as part of another group, refers to a straight or branched chain saturated hydrocarbon group having 1, 2, 3, 4, 5, 6 or 7 carbon atom(s). Representative examples of C₁₋₇ alkyl include, but are not limited to, methyl, ethyl, n-propyl, iso-propyl, n-butyl, iso-butyl, sec-butyl, tert-butyl, n-pentyl, iso-pentyl and n-hexyl. The term “C₁₋₃ alkyl” refers to a preferred embodiment of “C₁₋₇ alkyl” having 1, 2 or 3 carbon atoms.

The term “C₂₋₇ alkenyl”, as employed herein as such or as part of another group, refers to an aliphatic hydrocarbon group having 2 to 7 carbon atoms and containing at least one carbon to carbon double bond. Representative examples include, but are not limited to, ethenyl, prop-1-enyl, but-1-enyl, but-2-enyl, pent-1-enyl, pent-2-enyl, hex-1-enyl and hex-2-enyl.

The term “C₃₋₁₀ cycloalkyl”, as employed herein as such or as part of another group, refers to a saturated or partially saturated, monocyclic, bicyclic or polycyclic hydrocarbon ring system having 3 to 10 carbon atoms. Representative examples of C₃₋₁₀ cycloalkyl include, but are not limited to, cyclopropyl, cyclobutyl, cyclopentyl and cyclohexyl, decahydronaphthalen-1-yl and octahydro-1H-inden-2-yl. The term “C₃₋₇ cycloalkyl”, as employed herein as such or as part of another group, refers to a saturated or partially saturated monocyclic hydrocarbon ring containing 3, 4, 5, 6 or 7 carbon atoms.

The term “C₁₋₇ alkoxy”, as employed herein as such or as part of another group, refers to C₁₋₇ alkyl, as defined herein, appended to the parent molecular moiety through an oxygen atom. Representative examples of C₁₋₇ alkoxy include, but are not limited to methoxy, ethoxy, propoxy, butoxy, isobutoxy, sec-butoxy and tert-butoxy.

The term “halo” or “halogen”, as employed herein as such or as part of another group, refers to chlorine, bromine, fluorine or iodine.

The term “amino”, as employed herein as such or as part of another group, refers to an —NH₂ group. The term “hydroxy”, as employed herein as such or as part of another group, refers to an —OH group. The term “cyano”, as employed herein as such or as part of another group, refers to a —CN group. The term “carboxy”, as employed herein as such or as part of another group, refers to —COOH group. The term “carbonyl”, as employed herein as such or as part of another group, refers to a carbon atom double-bonded to an oxygen atom (C═O). The term “oxo”, as employed herein as such or as part of another group, refers to oxygen atom linked to another atom by a double bond (═O).

The term “hydroxy C₁₋₇ alkyl”, as employed herein, refers to at least one hydroxy group, as defined herein, appended to the parent molecular moiety through a C₁₋₇ alkyl group, as defined herein. Representative examples include, but are not limited to, hydroxymethyl, 2,2-dihydroxyethyl, 1-hydroxyethyl, 3-hydroxypropyl, 1-hydroxypropyl, 1-methyl-1-hydroxyethyl and 1-methyl-1-hydroxypropyl.

The term “halo C₁₋₇ alkyl”, as employed herein, refers to at least one halogen, as defined herein, appended to the parent molecular moiety through a C₁₋₇ alkyl group, as defined herein. Representative examples include, but are not limited to, fluoromethyl, difluoromethyl, trifluoromethyl, 2-chloroethyl and 3-bromopropyl.

The term “aryl”, as employed herein, refers to a monocyclic, bicyclic or polycyclic aromatic hydrocarbon ring system of 6 to 14 carbon atoms. Examples of aryl groups include, but are not limited to phenyl, naphthyl, biphenyl, anthryl, tetrahydronaphthyl, fluorenyl, indanyl, biphenylenyl and acenaphthyl. Preferred aryl group is phenyl.

The term “aryl C₁₋₇ alkyl”, as employed herein, refers to at least one aryl group appended to the parent molecular moiety through a C₁₋₇ alkyl group, as defined herein. Examples of aryl C₁₋₇ alkyl groups include, but are not limited to benzyl, benzhydryl, 1-phenylethyl, 2-phenylethyl, 3-phenylpropyl, 2-phenylpropyl, 1-naphthylmethyl and 2-naphthylmethyl. Preferred aryl C₁₋₇ alkyl group is phenyl C₁₋₇ alkyl.

The term “heterocyclyl” includes the definitions of“heterocycloalkyl” and “heteroaryl”.

The term “heterocycloalkyl” refers to a non-aromatic, saturated or partially saturated, monocyclic or polycyclic ring system with 3 to 10 ring atoms of which at least one, preferably 1-4, is a heteroatom selected from the group consisting of O, N, and S.

One particular embodiment of “heterocycloalkyl” is a non-aromatic, saturated or partially saturated, monocyclic or polycyclic ring system with 5 to 10 ring atoms of which 1-4 are heteroatoms selected from the group consisting of N, O and S. Examples of heterocycloalkyl groups include piperidinyl, piperazinyl, pyrrolidinyl, morpholinyl, thiomorpholinyl, 1,3-dioxolanyl, tetrahydro-2H-pyran and 1,4-dioxanyl.

The term “heteroaryl” refers to a monocyclic, bicyclic, or polycyclic aromatic ring system of 5-14 ring atoms containing at least one, preferably 1 to 4, heteroatom selected from the group consisting of N, O and S. One particular embodiment of “heteroaryl” is a monocyclic, bicyclic, or polycyclic aromatic ring with 5-10 ring atoms of which 1-4 are heteroatoms selected from the group consisting of N, O and S. Examples of 5-10 membered heteroaryl groups include furan, thiophene, indole, azaindole, oxazole, thiazole, thiadiazole, isoxazole, isothiazole, imidazole, 1H-indazole, pyridine, pyrimidine, pyrazine, pyrrole, pyrazole, 1,3,4-oxadiazole, 1,2,4-triazole, 1H-tetrazole, 1,2,3,4-tetrahydroisoquinoline, benzoxazole, benzothiazole, benzofuran, benzisoxazole, benzimidazole, 3,4-dihydroisoquinolin-1(2H)-one, azabenzimidazole, indazole, quinazoline, quinoline, and isoquinoline. Examples of bicyclic heteroaryl groups include those where a phenyl, pyridine, pyrimidine or pyridazine ring is fused to a 5 or 6-membered monocyclic heterocyclyl ring having one or two nitrogen atoms in the ring, one nitrogen atom together with either one oxygen or one sulphur atom in the ring, or one O or S ring atom.

The term “heterocyclyl C₁₋₇ alkyl” refers to at least one heterocyclyl group, as defined above, appended to the parent molecular moiety through a C₁₋₇ alkyl group, as defined herein. Representative examples include, but are not limited to pyrrolidinyl-1-ethyl, pyrrolidinyl-1propyl or piperidinyl-1propyl.

The term “4-12 membered monocyclic or bicyclic ring containing 0-3 heteroatoms” refers to a monocyclic or bicyclic aromatic or non-aromatic cyclic ring having 4-12 ring member atoms of which 0-3 have been independently replaced with N or O. Representative examples of such rings include, but are not limited to phenyl, pyridine, pyrimidine, morpholine, piperidine, piperazine, imidazole, pyrazole, pyrrole, thiophene, cyclopropyl, 2,3dihydrobenzo[b][1,4]dioxine, 1,2,3,4-tetrahydroisoquinoline, quinoline, indazole, [1,2,4]triazolo[4,3-a]pyridine and tetrahydroisoquinoline. A particular embodiment of “4-12 membered monocyclic or bicyclic ring containing 0-3 heteroatoms” is a monocyclic or bicyclic aromatic or non-aromatic cyclic ring with 5-10 ring atoms of which 0-3 are heteroatoms selected from a group consisting of N or O.

The term “5-10 membered heterocyclic ring having 1-4 heteroatoms selected from O or N” refers to aromatic, saturated or partially saturated monocyclic, bicyclic or polycyclic ring which have 5 to 10 ring member atoms of which 1 to 4 are heteroatoms selected from a group consisting of O or N.

The term “optionally substituted”, if not otherwise specified, means that one, two or three hydrogen atoms of the optionally substituted group has been substituted with suitable groups as exemplified but not limited to C₁₋₇ alkyl, C₂₋₇ alkenyl, C₁₋₇ alkoxy, C₂₋₇ alkynyl, aryl, amido, amino, carboxy, cyano, C₃₋₁₀ cycloalkyl, halogen, hydroxy, nitro, halo C₁₋₇ alkyl, halo C₁₋₇ alkoxy, heterocyclyl, oxo(═O), thio(═S), —C(O)C₁₋₇ alkyl, —C(O)(aryl), —C(O)C₃₋₁₀ cycloalkyl, —C(O)(heterocyclyl), or two substituents on the same carbon atom is combined together to form an optionally substituted 3-8 member ring containing 0-3 heteroatoms independently selected form N, O and S. One particular embodiment of “optionally substituted” is 1-3 substituents selected from the group consisting of C₁₋₇ alkyl, C₃₋₇ cycloalkyl, halogen, nitro, cyano, amino, hydroxy, halo C₁₋₇ alkyl, hydroxy C₁₋₇ alkyl, C₁₋₇ alkoxy and halo C₁₋₇ alkoxy substituents.

The term “stereoisomers” refers to any enantiomers, diastereoisomers, or geometrical isomers of the compounds of the invention including those according to formula (I), (IA), (IB) or (IC), wherever they are chiral or when they bear one or more double bonds. When the compounds of the invention are chiral, they can exist in racemic or in optically active form Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, E and Z isomers as well as the appropriate mixtures thereof. Additionally, compounds may exist as tautomers, including keto-enol tautomers; all tautomeric isomers are provided by this invention.

The term “pharmaceutically acceptable salt” refers to the salts of the compounds, that is pharmaceutically acceptable and that possesses the desired pharmacological activity of the parent compound. Pharmaceutically acceptable salts of the compounds of this invention include those derived from suitable inorganic and organic acids and bases. Such salts include acid addition salts, formed with inorganic acids such as hydrochloric acid, hydrobromic acid, sulphuric acid, nitric acid, phosphoric acid, and the like; or formed with organic acids such as acetic acid, propionic acid, hexanoic acid, cyclo-pentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methane sulfonic acid, ethane sulfonic acid, 1,2-ethane-disulfonic acid, 2-hydroxyethanesulfonic acid, benzene sulfonic acid, 4-chlorobenzenesulfonic acid, 2-naphthalenesulfonic acid, 4-toluene-sulfonic acid, camphor sulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulphuric acid, gluconic acid, glutamic acid, hydroxyl naphthoic acid, salicylic acid, stearic acid, muconic acid, and the like.

In one embodiment, the compounds of the present invention are used in the treatment and/or prevention of diseases and/or disorders in which aberrant, abnormal or deregulated activity of bromodomain containing proteins contribute to the pathology and/or symptomology of such diseases and/or disorders.

In another particular embodiment, the compounds of the present invention are used in the treatment and/or prevention of diseases and/or disorders in which aberrant, abnormal or deregulated activity of BET family of bromodomain containing proteins; in particular BRD2, BRD3, BRD4 and BRD-t proteins, contribute to the pathology and/or symptomology of such diseases and/or disorders.

Bromodomain inhibitors are believed to be useful in the treatment of a variety of diseases or conditions related to systemic or tissue inflammation, inflammatory responses to infection or hypoxia, cellular activation and proliferation, lipid metabolism, fibrosis and in the prevention and treatment of viral infections.

Bromodomain inhibitors may be useful in the treatment of a wide variety of chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), asthma, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, depression, retinitis, uveitis, scleritis, hepatitis, pancreatitis, primary biliary cirrhosis, sclerosing cholangitis, Addison's disease, hypophysitis, thyroiditis, type I diabetes and acute rejection of transplanted organs.

Bromodomain inhibitors may be useful in the treatment of a wide variety of acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritisnodosa, Behcet's disease, Kawasaki disease, Takayasu's Arteritis, vasculitis with organ involvement and acute rejection of transplanted organs.

Bromodomain inhibitors may be useful in the prevention or treatment of diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus.

Bromodomain inhibitors may be useful in the prevention or treatment of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

Bromodomain inhibitors may be useful in the treatment of disorders of lipid metabolism via the regulation of APO-A1 such as hypercholesterolemia, atherosclerosis and Alzheimer's disease.

Bromodomain inhibitors may be useful in the treatment of fibrotic conditions such as idiopathic pulmonary fibrosis, renal fibrosis, post-operative stricture, keloid formation, scleroderma and cardiac fibrosis.

Bromodomain inhibitors may be useful in the prevention and treatment of viral infections such as herpes virus, human papilloma virus, adenovirus and poxvirus and other DNA viruses. Bromodomain inhibitors may be useful in the treatment of cancer, including hematological, epithelial including lung, breast and colon carcinomas, midline carcinomas, mesenchymal, hepatic, renal and neurological tumors.

In one embodiment the disease or condition for which a bromodomain inhibitor is indicated is selected from diseases associated with systemic inflammatory response syndrome, such as sepsis, burns, pancreatitis, major trauma, haemorrhage and ischaemia.

In this embodiment the bromodomain inhibitor would be administered at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality.

In another embodiment the bromodomain inhibitor would be administered prior to surgical or other procedures associated with a high risk of sepsis, haemorrhage, extensive tissue damage, SIRS or MODS (multiple organ dysfunction syndrome).

In a particular embodiment the disease or condition for which a bromodomain inhibitor is indicated is sepsis, sepsis syndrome, septic shock and endotoxaemia. In another embodiment, the bromodomain inhibitor is indicated for the treatment of acute or chronic pancreatitis. In another embodiment the bromodomain is indicated for the treatment of burns. In one embodiment the disease or condition for which a bromodomain inhibitor is indicated is selected from herpes simplex infections and reactivations, cold sores, herpes zoster infections and reactivations, chickenpox, shingles, human papilloma virus, cervical neoplasia, adenovirus infections, including acute respiratory disease, poxvirus infections such as cowpox and smallpox and African swine fever virus. In one particular embodiment a bromodomain inhibitor is indicated for the treatment of Human papilloma virus infections of skin or cervical epithelia.

In yet another embodiment, compounds of the present invention inhibit one or more of BRD2, BRD3, BRD4, BRDT, and/or another member of the bromodomain-containing proteins, or a mutant thereof.

In yet another embodiment, compounds of the present invention inhibit two or more of BRD2, BRD3, BRD4, BRDT, and/or another member of the bromodomain-containing proteins, or a mutant thereof.

In yet another embodiment, compounds of the present invention are inhibitors of one of more of the bromodomain-containing proteins, such as BRD2, BRD3, BRD4, and/or BRDT and are therefore useful for treating one or more disorders associated with activity of one or more of the bromodomain-containing proteins, such as BRD2, BRD3, BRD4, and/or BRDT. Thus, in yet another embodiment, the present invention provides a method for treating an bromodomain-containing protein-mediated disorder, such as a BET-mediated, a BRD2-mediated, a BRD3-mediated, a BRD4-mediated disorder, and/or a BRDT-mediated disorder comprising the step of inhibiting a bromodomain-containing protein, such as a BET protein, such as BRD2, BRD3, BRD4, and/or BRDT, or a mutant thereof, by administering to a patient in need thereof a provided compound, or a pharmaceutically acceptable composition thereof.

The term “diseases or disorders where bromodomain inhibition is desired”, is intended to include each of or all of the above disease states.

While it is possible that for use in therapy, a compound of formula (I) as well as pharmaceutically acceptable salts thereof may be administered as such, it is common to present the active ingredient as a pharmaceutical composition.

The compounds and pharmaceutically compositions of the present invention may be used in combination with other drugs that are used in the treatment/prevention/suppression or amelioration of the diseases or conditions for which compounds of the present invention may be useful. Such other drugs may be administered, by a route and in an amount commonly used there for, simultaneously or sequentially with a compound of the present invention. When a compound of the present invention is used simultaneously with one or more other drugs, a pharmaceutical composition containing such other drugs in addition to the compound of the present invention may also be preferred. Accordingly, the pharmaceutical compositions of the present invention include those that also contain one or more other active ingredients, in addition to a compound of the present invention.

A pharmaceutical composition of the invention may be formulated as being compatible with its intended route of administration, which may preferably be an oral administration. For example the pharmaceutical compositions of the invention may be formulated for administration by inhalation, such as aerosols or dry powders; for oral administration, such in the form of tablets, capsules, gels, syrups, suspensions, emulsions, elixirs, solutions, powders or granules; for rectal or vaginal administration, such as suppositories; or for parenteral injection (including intravenous, subcutaneous, intramuscular, intravascular, or infusion) such as a sterile solution, suspension or emulsion.

The compounds of the present invention may also be entrapped in microcapsules prepared, for example, by coacervation techniques or by interfacial polymerization, for example, hydroxymethyl cellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The novel bicyclic heterocyclic derivatives of formula (I) according to the present invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. The details of the processes according to the present invention are provided in the example section below.

In a further aspect, the compounds of the present invention can also contain un-natural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses.

Exemplary isotopes that can be incorporated in to compounds of the invention include isotopes of hydrogen, carbon, nitrogen, oxygen, phosphorous, sulphur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the Schemes and/or in the Examples herein below, by substituting an isotopically labeled reagent for a non-isotopically labeled reagent.

The abbreviations used in the entire specification may be summarized herein below with their particular meaning.

MeOH—Methanol; EtOH—Ethanol; DME—1,2-dimethoxyethane; DCM—Dichloromethane; DMF—N,N-Dimethylformamide; DMSO—Dimethylsulfoxide; CDCl₃—Deuterated chloroform; EtOAc—Ethyl acetate; ACN—Acetonitrile; THF—Tetrahydrofuran; TEA—Triethylamine; DIPEA—Diisopropylethylamine; AcOH—Acetic acid; TBS-Cl—tert-Butyldimethylsilyl chloride; TBAF—Tetrabutylammonium fluoride; TMS—Trimethylsilyl; KCN—Potassium cyanide; NBS—N-bromo succinimide; NCS—N-chlorosuccinamide; NaOMe—Sodium ethoxide; H₂SO₄—Sulfuric acid; NaHCO₃—Sodium bicarbonate; Na₂CO₃—Sodium carbonate; Cs₂CO₃—Cesium carbonate; NaBH₄—Sodium borohydride; (BOC)₂O—Di-tert-butyldicarbonate; EDC.HCl—1-Ethyl-3-(3-dimethylamino propyl)carbodiimide.HCl; HOBt—1-Hydroxy-benzotriazole; HATU—1-[Bis(dimethylamino) methylene]-1H-1,2,3-triazolo[4,5-b]-pyridinium-3-oxidhexafluorophosphate; PyBOP—(Benzotriazol-1-yloxy)tripyrrolidino-phosphoniumhexafluorophosphate; POCl₃— Phosphorous oxychloride; AcCl—Acetyl chloride; NaOH—Sodium hydroxide; HCl—Hydrochloric acid; Pd (pph₃)₄—Tetrakis(tri-phenylphosphine)palladium (0); Fe—Iron powder; Pd/C—Palladium on activated carbon; H₂O—Water; Fe—Iron powder; h—Hour; N—Normality; M—Molarity; s—Singlet; d—Doublet; t—Triplet; m—Multiplet; TLC—Thin layer chromatography; ¹HNMR—Proton nuclear magnetic resonance; HPLC—High-performance liquid chromatography; MS—Mass spectroscopy; LC—Liquid chromatography; H—Proton; MHz—Megahertz; Hz—Hertz; Ppm—Parts per million; Bs—Broad singlet; ES—Electro spray; g—Gram; mmol—Millimol; mL—Millilitre; RT—Room temperature; δ—Chemical shift expressed in ppm.

Although the invention has been illustrated by following examples, it is not to be construed as being limited thereby. Various modifications and embodiments can be made without departing from the spirit and scope thereof. The MS data provided in the examples described below were obtained as follows: Mass spectrum: LC/MS Agilent 6120 Quadrapole LC/MS. The NMR data provided in the examples described below were obtained as follows: ¹H-NMR: Varian 400 MHz. The microwave chemistry was performed on a CEM Explorer.

The procedure for the compounds of formula (I) are detailed herein below stepwise including the general synthesis of various intermediates involved in process of synthesis of the compounds according to the present invention.

### EXAMPLES

### Intermediate-1: 6-(3, 5-Dimethylisoxazol-4-yl)-7-methoxy-2-oxo-1-(pyridin-2-yl-methyl)-1, 2-dihydroquinoline-3-carboxylic Acid

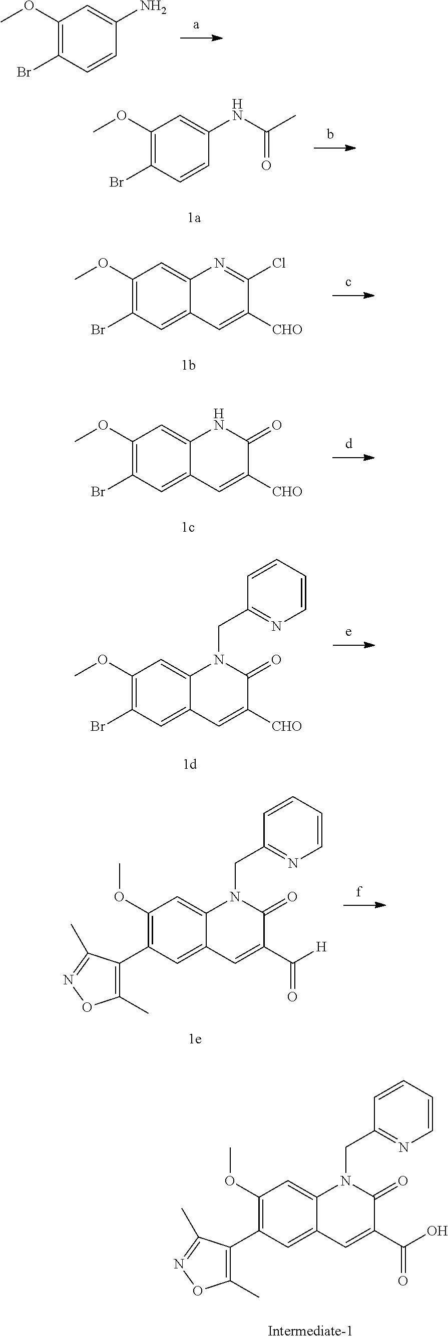

### Step-a: N-(4-bromo-3-methoxyphenyl)acetamide (1a)

To an ice-cooled solution of 4-bromo-3-methoxyaniline (2.0 g, 9.90 mmol) in DCM (25 mL) was added triethylamine (4.1 mL, 29.7 mmol) and, after being stirred for 5 min, acetylchloride (1.05 mL, 14.85 mmol). The reaction mixture was quenched by addition of aqueous NaHCO₃ solution (pH-8) followed by extraction with DCM (200 mL×2). The combined organic layers were washed with water (200 mL) and brine (200 mL), dried over sodium sulphate and concentrated under reduced pressure. The residue was directly used for next step without further purification (2.5 g). ¹H NMR (400 MHz, DMSO-d₆) δ 10.06 (s, 1H), 7.45-7.43 (m, 2H), 7.10 (dd, J₁=2.0 Hz, J₂=8.3 Hz, 1H), 3.79 (s, 3H), 2.04 (s, 3H); LC-MS: m/z 244.1 (M+H)⁺.

### Step-b: 6-Bromo-2-chloro-7-methoxyquinoline-3-carbaldehyde (1b)

POCl₃ (7.6 mL, 81.96 mmol) was added dropwise to DMF (2.5 mL, 32.78 mmol) at 0° C. followed by stirring for 5 min. Intermediate-1a (2.0 g, 8.19 mmol) was added and resulting solution was heated to 80° C. for 6 h. The mixture was cooled to RT, quenched with ice water and extracted with EtOAc (200 mL×2). The combined organic layers were washed with water (200 mL) and brine (200 mL), dried over sodium sulphate and concentrated under reduced pressure. The residue was directly used for next step without further purification (2.0 g). ¹H NMR (400 MHz, DMSO-d₆) δ 10.33 (s, 1H), 8.88 (s, 1H), 8.64 (s, 1H), 7.59 (s, 1H), 4.07 (s, 3H); LC-MS: m/z 300 (M+H)⁺.

### Step-c: 6-Bromo-7-methoxy-2-oxo-1,2-dihydroquinoline-3-carbaldehyde (1c)

A suspension of intermediate-1b (2.0 g, 6.65 mmol) in 70% acetic acid (40 mL) was heated to reflux for 6 h. Upon cooling the mixture to RT a solid product was precipitated out which was filtered and washed with water and dried under reduced pressure to afford the title compound as brown solid (1.5 g, 80%). ¹H NMR (400 MHz, DMSO-d₆) δ 12.18 (s, 1H), 10.17 (s, 1H), 8.42 (s, 1H), 8.22 (s, 1H), 6.93 (s, 1H), 3.94 (s, 3H); LC-MS: m/z 284 (M+2H)²⁺.

### Step-d: 6-Bromo-7-methoxy-2-oxo-1-(pyridin-2-ylmethyl)-1,2-dihydroquinoline-3-carbaldehyde (1d)

To a solution of intermediate-1c (9 g, 31.91 mmol) in DMF (80 mL) were added potassium carbonate (13.2 g, 95.73 mmol) followed by 2-(chloromethyl)pyridine hydrochloride (6.4 g, 35.1 mmol). The mixture was stirred at 80° C. for 16 h. The mixture was then diluted with water and extracted with EtOAc (400 mL×2). The combined organic layers were washed with water (400 mL) and brine (300 mL), dried over sodium sulphate and concentrated under reduced pressure. The residue was directly used for next step without further purification (7.5 g, 63%). ¹H NMR (400 MHz, DMSO-d₆) δ 10.25 (s, 1H), 8.51-8.48 (m, 2H), 8.31 (s, 1H), 7.78 (t, J=7.9 Hz, 1H), 7.40 (d, J=8.4 Hz, 1H), 7.31-7.28 (m, 1H), 7.13 (s, 1H), 5.70 (s, 2H), 3.86 (s, 3H); LC-MS: m/z 373.0 (M+H)+.

### Step-e: 6-(3,5-Dimethylisoxazol-4-yl)-7-methoxy-2-oxo-1-(pyridin-2-ylmethyl)-1,2-dihydro quinoline-3-carbaldehyde (1e)

To a solution of intermediate-1d (4.0 g, 10.72 mmol) in 1,4-dioxane (40 mL) and H₂O (10 mL) were added 3,5-dimethylisoxazoleboronic acid (2.30 g, 16.08 mmol), sodium carbonate (3.41 g, 32.16 mmol) followed by degassing with nitrogen purging for 20 min. Then tetrakistriphenylphosphine palladium (2.47 g, 2.14 mmol) was added and the mixture was heated at 100° C. for 8 h. The mixture was then concentrated under reduced pressure and the residue was diluted with EtOAc (200 ml), washed with water (200 mL) and brine (200 mL), dried over sodium sulphate and concentrated under reduced pressure. The residue was washed with hexane to give the title compound as yellow solid (3.2 g, 76%). ¹H NMR (400 MHz, DMSO-d₆) δ 10.28 (s, 1H), 8.54 (s, 1H), 8.52 (d, J=4.4 Hz, 1H), 7.94 (s, 1H), 7.82-7.77 (m, 1H), 7.44 (d, J=7.8 Hz, 1H), 7.33-7.29 (m, 1H), 7.17 (s, 1H), 5.72 (s, 2H), 3.81 (s, 3H), 2.27 (s, 3H), 2.08 (s, 3H); LC-MS: m/z 390.1 (M+H)+.

### Step-f: 6-(3, 5-Dimethylisoxazol-4-yl)-7-methoxy-2-oxo-1-(pyridin-2-ylmethyl)-1,2-dihydroquinoline-3-carboxylic Acid (intermediate-1)

To a solution of intermediate-1e (1.2 g, 3.08 mmol) in mixture of acetonitrile (12 mL) and H₂O (6 mL) were added sodiumdihydrogenphosphate (1.6 g), hydrogen-peroxide 30% (1 mL) and sodiumchlorite (0.85 g). The mixture was stirred at RT for 4 h. The mixture was then quenched with ice water, solids were separated, filtered, washed thoroughly with water and dried under reduced pressure to give the title compound as yellow solid (0.85 g, 75%). ¹H NMR (400 MHz, DMSO-d₆) δ 14.36 (s, 1H), 8.99 (s, 1H), 8.50 (d, J=4.4 Hz, 1H), 8.05 (s, 1H), 7.84-7.80 (m, 1H), 7.51 (d, J=7.8 Hz, 1H), 7.34-7.31 (m, 1H), 7.28 (s, 1H), 5.84 (s, 2H), 3.84 (s, 3H), 2.28 (s, 3H), 2.09 (s, 3H); LC-MS: m/z 406.2 (M+H)⁺.

### Intermediate-2: 3-Bromo-6-(3,5-dimethylisoxazol-4-yl)-7-methoxy-1-(pyridin-2-yl-methyl)quinolin-2(1H)-one

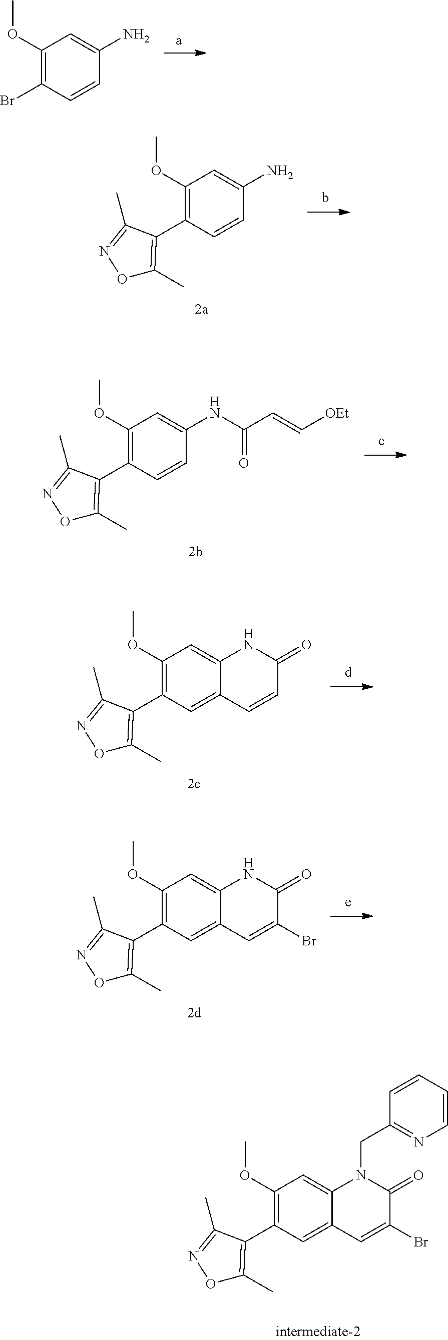

### Step-a: 4-(3,5-Dimethylisoxazol-4-yl)-3-methoxyaniline (2a)

To a solution of 4-bromo-3-methoxyaniline (1 g, 4.95 mmol) in DME (15 ml) and water (5 ml) were added Na₂CO₃ (1.6 g, 14.85 mmol) and 3,5-dimethylisoxazole-4-boronic acid (1.4 g, 9.90 mmol). The mixture was degassed with nitrogen for 15 min. Then Pd[PPh₃]₄(0.29 g, 0.24 mmol) was added followed by degassing with nitrogen for 5 min and heating to 90° C. for 16 h. The mixture was diluted with EtOAc (150 ml), washed with water (150 mL) and brine (150 mL), dried over sodium sulphate and concentrated under reduced pressure. The residue was purified on silica gel (60-120 mesh) to afford the title product as pale yellow solid (0.6 g, 60%). ¹H NMR (400 MHz, DMSO-d₆): δ 6.78 (d, J=7.8 Hz, 1H), 6.30-6.19 (m, 2H), 5.26 (s, 2H), 3.66 (s, 3H), 2.19 (s, 3H), 2.02 (s, 3H); LC-MS: m/z 219.2 (M+H)⁺

### Step-b: (E)-N-(4-(3,5-dimethylisoxazol-4-yl)-3-methoxyphenyl)-3-ethoxyacrylamide (2b)

To a solution of intermediate-2a (0.6 g, 4.12 mmol) in pyridine (5 mL) at 0° C. was added (E)-3-ethoxyacryloylchloride (0.83 g, 6.19 mmol). The mixture was allowed to stir at RT for 16 h. The mixture was quenched with ice water and diluted with EtOAc (100 ml), washed with 1N HCl (100 mL) and brine (100 mL), dried over sodium sulphate and concentrated under reduced pressure. The residue was purified by on silica gel (60-120 mesh) to afford the title product as pale yellow solid 0.4 g (46%). ¹H NMR (400 MHz, DMSO-d₆): δ 9.85 (s, 1H), 7.52-7.48 (m, 2H), 7.22-7.21 (m, 1H), 7.11-7.09 (m, 1H), 5.53 (d, J=12.2 Hz, 2H), 3.98-3.93 (m, 2H), 3.73 (s, 3H), 2.23 (s, 3H), 2.05 (s, 3H), 1.30-1.22 (m, 2H); LC-MS: m/z 317.2 (M+H)⁺.

### Step-c: 6-(3,5-Dimethylisoxazol-4-yl)-7-methoxyquinolin-2(1H)-one (2c)

A solution of intermediate-2b (0.4 g, 2.16 mmol) in H₂SO₄ (3 mL) was stirred at RT for 2 h. The mixture was quenched with ice water and the solid formed was filtered off, washed with water and dried under reduced pressure to afford the title product as off white solid (0.2 g, 59%). H NMR (400 MHz, DMSO-d₆): δ 11.76 (bs, 1H), 7.82 (d, J=9.3 Hz, 1H), 7.53 (s, 1H), 6.94 (s, 1H), 6.35 (d, J=9.8 Hz, 1H), 3.81 (s, 3H), 2.26 (s, 3H), 2.07 (s, 3H); LC-MS: m/z 271.1 (M+H)⁺.

### Step-d: 3-Bromo-6-(3,5-dimethylisoxazol-4-yl)-7-methoxyquinolin-2(1H)-one (2d)

To a cooled solution of intermediate-2c (3.5 g, 12.96 mmol) in DMF (30 mL) was added N-bromosuccinimide (2.8 g, 15.55 mmol) portion wise. The mixture was stirred at RT for 1 h. The mixture was quenched with ice water, solids were separated, filtered, washed thoroughly with water and dried under reduced pressure to afford the title compound (3.5 g, 78%); ¹H NMR (400 MHz, DMSO-d₆): δ 12.25 (s, 1H), 8.40 (s, 1H), 7.56 (s, 1H), 6.96 (s, 1H), 3.83 (s, 3H), 2.26 (s, 3H), 2.07 (s, 3H); LC-MS: m/z 351.1 (M+2H)²⁺.

### Step-e: 3-Bromo-6-(3,5-dimethylisoxazol-4-yl)-7-methoxy-1-(pyridin-2-yl-methyl)quinolin-2(1H)-one (intermediate-2)

To a solution of intermediate-2d (3.5 g, 10.02 mmol) in DMF (30 mL) were added potassium carbonate (4.2 g, 30.06 mmol) followed by 2-(chloromethyl)pyridine hydrochloride (2.5 g, 15.64 mmol). The mixture was stirred at RT for 16 h. The mixture was quenched with ice water, solids were separated, filtered, washed thoroughly with water and dried under reduced pressure to afford the title compound (3.0 g, 68%); δ 8.52 (s, 1H), 8.50 (s, 1H), 7.80-7.79 (m, 1H), 7.64 (s, 1H), 7.40 (d, J=7.8 Hz, 1H), 7.32-7.30 (m, 1H), 7.13 (s, 1H), 5.72 (s, 2H), 3.75 (s, 3H), 2.25 (s, 3H), 2.06 (s, 3H); LC-MS: m/z 440.1 (M+H)⁺.

The below intermediate was prepared according to the step-e of the above procedure by using suitable reactant and reagents in the presence of suitable reaction conditions.

| No. | Structure | LCMS: m/z |
|---|---|---|
| 3 | 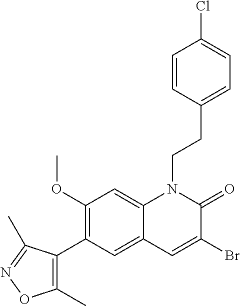 [SMILES: COc1cc2c(cc1-c1c(C)noc1C)cc(Br)c(=O)n2CCc1ccc(Cl)cc1] | ¹H NMR (400 MHz, DMSO-d₆): δ 8.44 (s, 1H), 7.62 (s, 1H), 7.39-7.30 (m, 4H), 6.98 (s, 1H), 4.60 (t, J = 7.3 Hz, 2H), 3.92 (s, 3H), 3.00 (t, J = 7.3 Hz, 2H), 2.27 (s, 3H), 2.08 (s, 3H); LCMS: m/z 487.0 (M + H)⁺. |

### Intermediate-4: (3-Methyl-4-(4,4,5,5-tetramethyl-1,3,2-dioxaborolan-2-yl) isoxazol-5-yl)methyl acetate

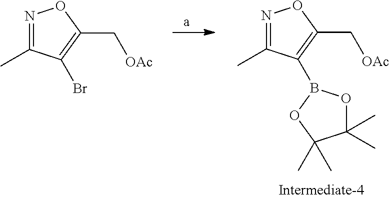

To a 200 mL flask (under N₂) was added dichlorobis(acetonitrile)palladium(II) (0.22 g, 0.85 mmol) and dicyclohexyl(2′,6′-dimethoxybiphenyl-2-yl)phosphine (1.40 g, 3.41 mmol). To the solid were sequentially added a solution of (4-bromo-3-methylisoxazol-5yl)methyl acetate (10.0 g, 42.73 mmol) in dry 1,4-dioxane (100 mL), dry Et₃N (17.82 mL, 128.20 mmol), and 4,4,5,5-tetramethyl-1,3,2-dioxaborolane (15.50 mL, 106.83 mmol). The flask was sequentially evacuated, purged under N₂, and repeated for 20 min. The mixture was heated to 110° C. (under N₂) for 16 h. The mixture was cooled to RT and filtered through celite pad followed by washing with EtOAc. The filtrate was concentrated under reduced pressure. The residue was used for further step without further purification (12.0 g). ¹H NMR (400 MHz, CDCl₃): δ 5.31 (s, 2H), 2.37 (s, 3H), 2.11 (s, 12H).

### Intermediate-5: 2-Morpholinoethylmethanesulfonate

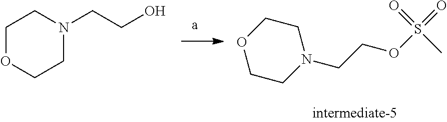

To an ice cooled solution of 2-morpholinoethan-1-ol (0.3 g, 2.28 mmol) in DCM (10 mL) were added triethylamine (1 mL, 6.86 mmol) followed by methanesulfonyl-chloride (0.21 mL, 2.74 mmol). The mixture was stirred at RT for 2 h. The mixture was diluted with DCM (50 mL), washed with water (50 mL) and brine (50 mL), dried over sodium sulphate and concentrated under reduced pressure. The residue (0.35 g) was used for further step without purification.

The below intermediates was prepared according to the above described procedure by using suitable reactant and reagents and in presence of suitable reaction conditions.

| No. | Structure | LCMS: m/z |
|---|---|---|
| 6 | 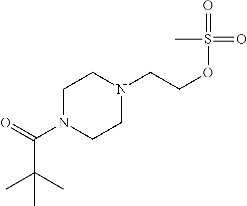 [SMILES: CC(C)(C)C(=O)N1CCN(CCOS(C)(=O)=O)CC1] | — |
| 7 | 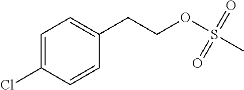 | 197.2 (M + H)⁺. |
| 8 | 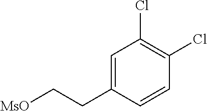 [SMILES: CS(=O)(=O)OCCc1ccc(Cl)c(Cl)c1] | ¹H NMR (400 MHz, CDCl₃): δ 7.40 (d, J = 8.4 Hz, 1H), 7.33 (d, J = 2.0 Hz, 1H), 7.09 (dd, J = 1.9 Hz, 8.3 Hz, 1H), 4.39 (t, J = 6.9 Hz, 2H), 3.02 (t, J = 6.9 Hz, 2H), 2.93 (s, 3H). |
| 9 | 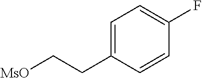 [SMILES: CS(=O)(=O)OCCc1ccc(F)cc1] | ¹H NMR (400 MHz, DMSO-d₆): δ 7.35-7.31 (m, 2H), 7.17-7.12 (m, 2H), 4.39 (t, J = 6.8 Hz, 2H), 3.11 (s, 3H), 2.94 (t, J = 6.8 Hz, 2H). |
| 10 | 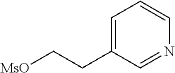 [SMILES: CS(=O)(=O)OCCc1cccnc1] | ¹H NMR (400 MHz, CDCl₃): δ 8.52 (dd, J = 1.5 Hz, 4.9 Hz, 1H), 8.51 (d, J = 2.0 Hz, 1H), 7.61-7.58 (m, 1H), 7.28 (dd, J = 4.4 Hz, 7.8 Hz, 1H), 4.43 (t, J = 6.9 Hz, 2H), 3.07 (t, J = 6.8 Hz, 2H), 2.92 (s, 3H). |
| 11 | 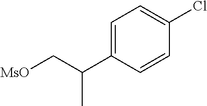 | ¹H NMR (400 MHz, CDCl₃): δ 7.32-7.29 (m, 2H), 7.18 (d, J = 8.3 Hz, 2H), 4.26-4.23 (m, 2H), 3.20- 3.14 (m, 1H), 2.87 (s, 3H), 1.34 (d, J = 7.4 Hz, 3H). |

### Intermediate-12: 6-Bromo-3-(hydroxydiphenylmethyl)-7-methoxy-1-(pyridin-2-yl-methyl)quinolin-2(1H)-one

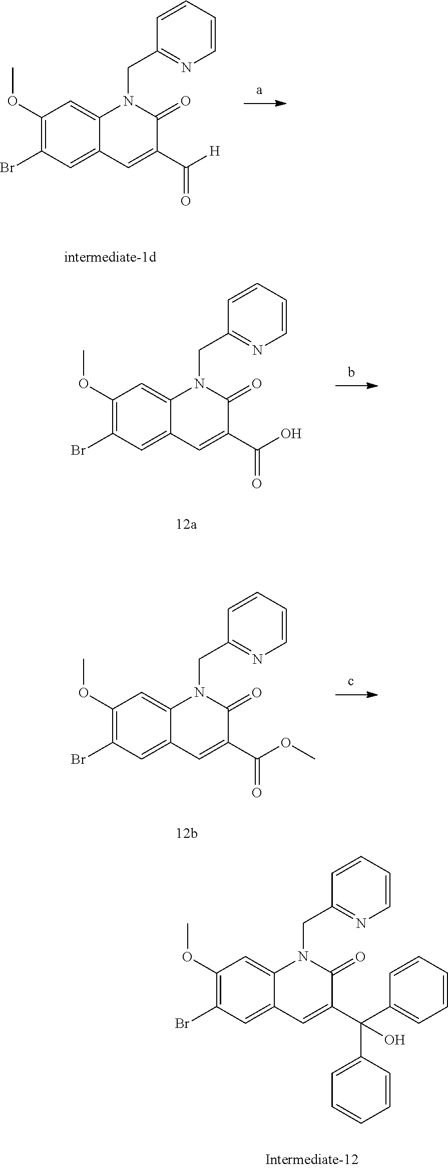 [SMILES: COc1cc2c(cc1Br)cc(C=O)c(=O)n2Cc1ccccn1 ; COc1cc2c(cc1Br)cc(C(=O)O)c(=O)n2Cc1ccccn1 ; COC(=O)c1cc2cc(Br)c(OC)cc2n(Cc2ccccn2)c1=O ; COc1cc2c(cc1Br)cc(C(O)(c1ccccc1)c1ccccc1)c(=O)n2Cc1ccccn1]

### Step-a: 6-Bromo-7-methoxy-2-oxy-2-oxo-1-(pyridin-2-ylmethyl)-1,2-dihydroquinoline-3-carboxylic Acid (12a)

To a solution of intermediate-1d (0.2 g, 0.71 mmol) in a mixture of acetonitrile (3 mL) and H₂O (1 mL) were added sodiumdihydrogenphosphate (0.28 g), hydrogen-peroxide 30% (0.2 mL) and sodiumchlorite (0.14 g). The mixture was stirred at RT for 16 h. The mixture was then quenched with ice water, solids were separated, filtered, washed thoroughly with water and vacuum dried to give the title compound as a yellow solid (0.15 g). ¹H NMR (400 MHz, DMSO-d₆) δ 14.26 (bs, 1H), 8.93 (s, 1H), 8.47 (d, J=4.4 Hz, 1H), 8.42 (s, 1H), 7.82-7.78 (m, 1H), 7.46 (d, J=7.8 Hz, 1H), 7.32-7.29 (m, 1H), 7.24 (s, 1H), 5.81 (s, 2H), 3.88 (s, 3H); LC-MS: m/z 389.2 (M+H)⁺.

### Step-b: Methyl 6-bromo-7-methoxy-2-oxo-1-(pyridin-2-ylmethyl)-1,2-dihydroquinoline-3-carboxylate (12b)

To a solution of intermediate-12a (1.0 g, 2.57 mmol) in DMF (20 mL) were added potassium carbonate (0.71 g, 5.14 mmol) followed by methyl iodide (0.31 mL, 5.14 mmol). The mixture was stirred at RT for 4 h. The mixture was diluted with water and extracted with EtOAc (200 mL×2). The combined organic layers were washed with water (200 mL) and brine (100 mL), dried over sodium sulphate and concentrated under reduced pressure. The residue was directly used for next step without further purification (0.8 g). ¹H NMR (400 MHz, DMSO-d₆) δ 8.52 (s, 1H), 8.49 (d, J=5.4 Hz, 1H), 8.48 (s, 1H), 7.79-7.74 (m, 1H), 7.34-7.27 (m, 2H), 7.09 (s, 1H), 5.65 (s, 2H), 3.83 (s, 3H), 3.81 (s, 3H); LC-MS: m/z 405.2 (M+2H)²⁺.

### Step-c: 6-Bromo-3-(hydroxydiphenylmethyl)-7-methoxy-1-(pyridin-2-ylmethyl)-quinolin-2(1H)-one (Intermediate-12)

To an ice-cooled solution of intermediate-12b (0.5 g, 1.24 mmol) in THF (15 mL) was added phenyl magnesium bromide (3.7 mL, 3.72 mmol). The reaction mixture was then allowed to stir at RT for 3 h. The mixture was quenched with aqueous ammonium chloride and extracted with EtOAc (100 mL×2). The combined organic layers were washed with water (100 mL) and brine (100 mL), dried over sodium sulphate and concentrated under reduced pressure. The residue obtained was purified by silica gel (60-120 mesh) column chromatography (elution 1% MeOH-DCM) to give the title compound (0.08 g, 12%). ¹H NMR (400 MHz, DMSO-d₆) δ 8.49 (d, J=4.9 Hz, 1H), 8.0 (s, 1H), 7.77-7.76 (m, 1H), 7.35-7.26 (m, 12H), 7.21 (d, J=7.8 Hz, 1H), 7.15 (s, 1H), 6.73 (s, 1H), 5.65 (s, 2H), 3.81 (s, 3H).

### Intermediate-13: 5-Bromo-4-methoxy-2-nitrobenzaldehyde

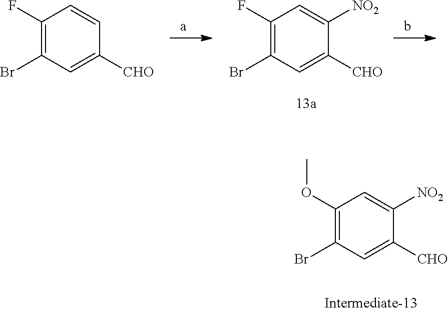

### Step-a: 5-Bromo-4-fluoro-2-nitrobenzaldehyde 13a

To a cooled solution of 3-bromo-4-fluorobenzaldehyde (15.0 g, 73.8 mmol) in H₂SO₄ (150 mL) was added HNO₃ (10 mL) dropwise followed by stirring at RT for 3 h. The mixture was poured into crushed ice. The solids were filtered, washed thoroughly with water and dried under reduced pressure to afford the title compound (11 g, 61%); ¹H NMR (400 MHz, DMSO-d₆): δ 10.15 (s, 1H), 8.32 (d, J=8.3 Hz, 1H), 8.26 (d, J=6.9 Hz, 1H).

### Step-b: 5-Bromo-4-methoxy-2-nitrobenzaldehydeintermediate (intermediate-13)

To an ice cooled solution of intermediate-13a (2.5 g, 10.08 mmol) in MeOH (30 mL) was added sodium methoxide (0.8 g, 15.1 mmol) followed by stirring at RT for 8 h. The mixture was quenched with ice water. Solids were separated, filtered, washed thoroughly with water and dried under reduced pressure. The product was further recrystallized by using 10% EtOAc-hexane to afford the title compound (1.0 g, 40%). ¹H NMR (400 MHz, DMSO-d₆): δ 10.04 (s, 1H), 8.17 (s, 1H), 7.80 (s, 1H), 4.06 (s, 3H); LC-MS: m/z 260 (M+H)⁺.

### Intermediate-14: 4-((tert-Butyldimethylsilyl)oxy)-3-fluoroaniline

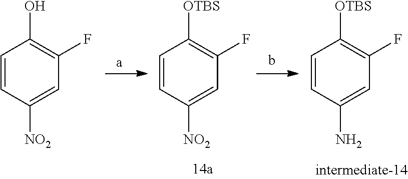 [SMILES: O=[N+]([O-])c1ccc(O)c(F)c1 ; CC(C)(C)[Si](C)(C)Oc1ccc([N+](=O)[O-])cc1F ; CC(C)(C)[Si](C)(C)Oc1ccc(N)cc1F]

### Step-a: tert-Butyl (2-fluoro-4-nitrophenoxy)dimethylsilane 14a

To a cooled solution of 2-fluoro-4-nitrophenol (1.0 g, 6.36 mmol) in DCM (15 mL) was added imidazole (1.4 g, 9.54 mmol) followed by TBS-Cl (0.9 g, 12.72 mmol). The mixture was stirred at RT for 5 h. The mixture was diluted with DCM (250 mL), washed with water (250 mL) and brine (250 mL), dried over sodium sulphate and concentrated under reduced pressure. The residue was used in next step without purification (0.5 g, 30%). ¹H NMR (400 MHz, CDCl₃): δ 8.0-7.95 (m, 2H), 7.02-6.97 (m, 1H), 1.01 (s, 9H), 0.25 (s, 6H).

### Step-b: 4-((tert-Butyldimethylsilyl) oxy)-3-fluoroaniline (Intermediate-14)

To a solution of intermediate-14a (0.4 g, 1.47 mmol) in EtOH (5 mL), THF (2.5 mL) and H₂O (1 mL) were added iron powder (0.2 g, 3.67 mmol) and NH₄Cl (0.22 g, 4.41 mmol). The mixture was heated to reflux for 1 h. The mixture was cooled to RT, filtered through celite followed by washing with EtOAc. The combined filtrate was concentrated under reduced pressure. The residue was diluted with water, extracted with EtOAc (100 mL), washed with brine (100 mL), dried over sodium sulphate and concentrated under reduced pressure to afford the title compound as brown oil (0.2 g). LC-MS: m/z 242.1 (M+H)⁺.

The below intermediates were prepared according to the procedure depicted above by using suitable reactants and reagents and appropriate reaction conditions.

| No. | Structure | LCMS: m/z |
|---|---|---|
| 15 | 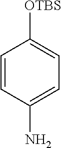 [SMILES: CC(C)(C)[Si](C)(C)Oc1ccc(N)cc1] | 224.2 (M + H)⁺ |
| 16 | 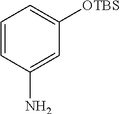 [SMILES: CC(C)(C)[Si](C)(C)Oc1cccc(N)c1] | 224.2 (M + H)⁺ |
| 17 | 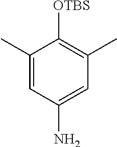 | 252.2 (M + H)⁺ |

### Intermediate-18: 3-(Azetidine-1-carbonyl)-6-bromo-7-methoxyquinolin-2(1H)-one

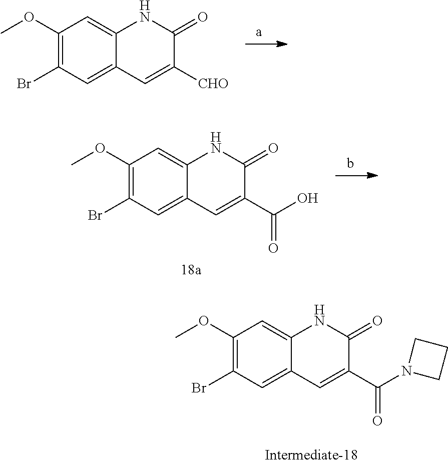 [SMILES: COc1cc2[nH]c(=O)c(C=O)cc2cc1Br ; COc1cc2[nH]c(=O)c(C(=O)O)cc2cc1Br ; COc1cc2[nH]c(=O)c(C(=O)N3CCC3)cc2cc1Br]

### Step-a: 6-Bromo-7-methoxy-2-oxo-1,2-dihydroquinoline-3-carboxylic acid 18a

The process of this step was adopted from step-f of intermediate-1. ¹H NMR (400 MHz, DMSO-d₆) δ 13.80 (bs, 2H), 8.83 (s, 1H), 8.33 (s, 1H), 7.04 (s, 1H), 3.96 (s, 3H); LC-MS: m/z 300.0 (M+2H)²⁺

### Step-b: 3-(Azetidine-1-carbonyl)-6-bromo-7-methoxyquinolin-2(1H)-one (Intermediate-18)

To a solution of intermediate-18a (3.0 g, 10.1 mmol) in DMF (30 mL) were added azetidine hydrochloride (1.42 g, 15.15 mmol), HOBt (2.7 g, 20.2 mmol), EDC.HCl (3.9 g, 20.2 mmol) and triethylamine (2.7 mL, 20.2 mmol). The mixture was stirred at RT for 16 h. The mixture was then diluted with EtOAc (100 mL), washed with water (100 mL) and brine (100 mL), dried over sodium sulphate and concentrated under reduced pressure to afford the title compound as off white solid (2.5 g). ¹H NMR (400 MHz, DMSO-d₆) δ 11.98 (s, 1H), 8.04 (d, J=2.9 Hz, 2H), 6.91 (s, 1H), 4.04 (t, J=7.4 Hz, 2H), 3.98 (t, J=7.8 Hz, 2H), 3.90 (s, 3H), 2.22 (t, J=7.8 Hz, 2H); LC-MS: m/z 339.0 (M+2H)²⁺.

### Intermediate-19: 1-(6-(3,5-Dimethylisoxazol-4-yl)-7-methoxy-2-oxo-1-(pyridin-2-ylmethyl)-1,2-dihydroquinolin-3-yl)-2,2,2-trifluoroethyl Methanesulfonate

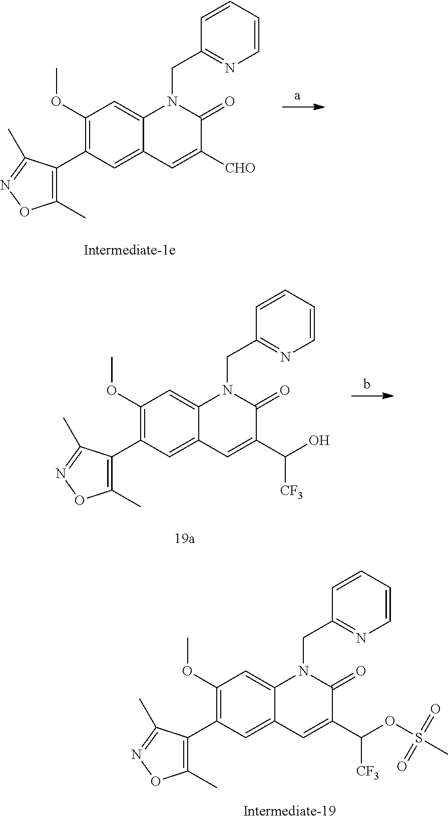 [SMILES: COc1cc2c(cc1-c1c(C)noc1C)cc(C=O)c(=O)n2Cc1ccccn1 ; COc1cc2c(cc1-c1c(C)noc1C)cc(C(O)C(F)(F)F)c(=O)n2Cc1ccccn1 ; COc1cc2c(cc1-c1c(C)noc1C)cc(C(OS(C)(=O)=O)C(F)(F)F)c(=O)n2Cc1ccccn1]

### Step-a: 6-(3,5-Dimethylisoxazol-4-yl)-7-methoxy-1-(pyridin-2-ylmethyl)-3-(2,2,2-trifluoro-1-hydroxyethyl)quinolin-2(1H)-one (19a)

To a cooled solution of intermediate-1e (0.02 g, 0.051 mmol) in THF (1 mL) were added tetrabutylammoniumfluoride 1.0 M in THF (0.015 mL, 0.015 mmol) and TMS-CF₃ (0.01 mL, 0.061 mmol) followed by stirring at 0° C. for 1 h. The mixture was quenched with saturated NH₄Cl and extracted with EtOAc (50 mL), washed with water (50 mL), dried over sodium sulphate and concentrated under reduced pressure. The residue was purified on preparative TLC to afford the title compound as an off white solid 0.01 g (43%). ¹H NMR (400 MHz, DMSO-d₆): δ 8.52 (d, J=3.9 Hz, 1H), 8.19 (s, 1H), 7.80-7.77 (m, 2H), 7.33-7.28 (m, 2H), 7.15 (s, 1H), 6.90 (d, J=7.4 Hz, 1H), 5.77-5.64 (m, 2H), 5.49-5.43 (m, 1H), 3.76 (s, 3H), 2.25 (s, 3H), 2.07 (s, 3H); LC-MS: m/z 460.2 (M+H)⁺.

### Step-b: 1-(6-(3,5-Dimethylisoxazol-4-yl)-7-methoxy-2-oxo-1-(pyridin-2-yl-methyl)-1,2-dihydro quinolin-3-yl)-2,2,2-trifluoroethyl Methanesulfonate (Intermediate-19)

The process of this step was adopted from step-a of intermediate-5. ¹H NMR (400 MHz, DMSO-d₆): δ 8.51 (d, J=4.4 Hz, 1H), 8.34 (s, 1H), 7.86 (s, 1H), 7.80-7.79 (m, 1H), 7.35-7.30 (m, 2H), 7.18 (s, 1H), 6.48-6.46 (m, 1H), 5.73 (s, 2H), 3.78 (s, 3H), 3.39 (s, 3H), 2.26 (s, 3H), 2.06 (s, 3H); LC-MS: m/z 538.1 (M+H)⁺.

### Intermediate-20: 6-(3, 5-Dimethylisoxazol-4-yl)-3-(hydroxymethyl)-7-methoxy-1-(pyridin-2-ylmethyl) quinolin-2(1H)-one

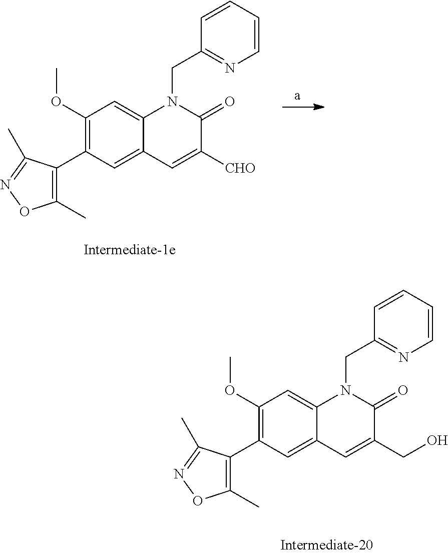 [SMILES: COc1cc2c(cc1-c1c(C)noc1C)cc(C=O)c(=O)n2Cc1ccccn1 ; COc1cc2c(cc1-c1c(C)noc1C)cc(CO)c(=O)n2Cc1ccccn1]

To an ice cooled solution of 6-(3,5-dimethylisoxazol-4-yl)-7-methoxy-2-oxo-1-(pyridin-2-ylmethyl)-1,2-dihydroquinoline-3-carbaldehyde (intermediate-1e) (0.07 g, 0.18 mmol) in MeOH (3 mL) was added NaBH₄ (0.007 g, 0.18 mmol) pinch wise followed by stirring at 0° C. for 1 h. The mixture was concentrated in vacuum. The residue was diluted with aqueous ammonium chloride and extracted with EtOAc (50 mL×2). The organic layer was washed with water (100 mL) and brine (100 mL), dried over sodium sulphate and concentrated under reduced pressure. The residue was purified on silica gel (100-200 mesh) to afford the title product as white solid 0.02 g (28%). ¹H NMR (400 MHz, DMSO-d₆): δ 8.52 (d, J=8.4 Hz, 1H), 7.91 (s, 1H), 7.77 (t, J=7.8 Hz, 1H), 7.62 (s, 1H), 7.32-7.28 (m, 2H), 7.11 (s, 1H), 5.75 (s, 2H), 5.27 (t, J=5.4 Hz, 1H), 4.46 (d, J=5.4 Hz, 2H), 3.73 (s, 3H), 2.25 (s, 3H), 2.06 (s, 3H); LC-MS: m/z 392.1 (M+H)⁺.

The below intermediates were prepared according to the procedure depicted above by using suitable reactants and reagents at appropriate reaction conditions.

| No. | Starting Compound | Intermediate formed | Characterization Data |
|---|---|---|---|
| 21 | 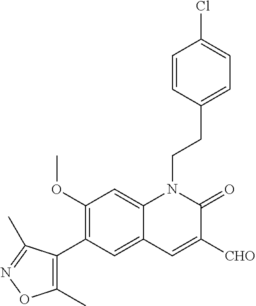 [SMILES: COc1cc2c(cc1-c1c(C)noc1C)cc(C=O)c(=O)n2CCc1ccc(Cl)cc1] | 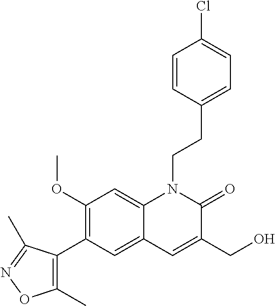 [SMILES: COc1cc2c(cc1-c1c(C)noc1C)cc(CO)c(=O)n2CCc1ccc(Cl)cc1] | ¹H NMR (400 MHz, CDCl₃) δ 7.64 (s, 1H), 7.32-7.21 (m, 5H), 6.73 (s, 1H), 4.67-4.62 (m, 2H), 4.52 (t, J = 7.8 Hz, 2H), 3.86 (s, 3H), 3.35-3.30 (m, 1H), 3.08 (t, J = 7.8 Hz, 2H), 2.31 (s, 3H), 2.16 (s, 3H); LC-MS: m/z 439.1 (M + H)⁺. |
| | Intermediate 21.1 | | |
| 21a | 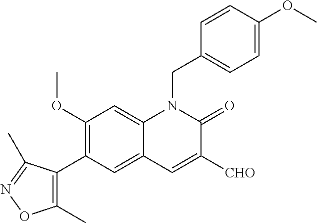 [SMILES: COc1ccc(Cn2c(=O)c(C=O)cc3cc(-c4c(C)noc4C)c(OC)cc32)cc1] | 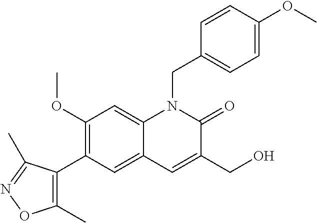 [SMILES: COc1ccc(Cn2c(=O)c(CO)cc3cc(-c4c(C)noc4C)c(OC)cc32)cc1] | ¹H NMR (400 MHz, DMSO- d₆): δ 7.89 (s, 1H), 7.67 (s, 1H), 7.30 (d, J = 8.3 Hz, 2H), 7.03 (s, 1H), 6.90 (d, J = 8.8 Hz, 2H), 5.53 (s,2H), 5.28 (t, J = 5.4 Hz, 1H), 4.87 (d, J = 5.3 Hz, 2H), 3.78 (s, 3H), 3.70 (s, 3H), 2.24 (s, 3H), 2.06 (s, 3H); LC-MS: m/z 421.2 (M + H)⁺. |
| | Intermediate-21a.1 | | |

The starting compounds 21.1 & 21a.1 were prepared according to the procedure depicted in step-d and step-e of intermediate-1 by using 4-chlorophenethylmethane-sulfonate and 1-(chloromethyl)-4-methoxybenzene as the reactant by using suitable reagents and solvents under appropriate reaction conditions. The characterization data for intermediate 21.1 & 21a.1 are given below. Intermediate-21.1: ¹H NMR (400 MHz, CDCl₃) δ 10.45 (s, 1H), 8.33 (s, 1H), 7.31 (s, 1H), 7.28-7.21 (m, 4H), 6.67 (s, 1H), 4.53 (t, J=7.9 Hz, 2H), 3.88 (s, 3H), 3.10 (t, J=7.8 Hz, 2H), 2.31 (s, 3H), 2.15 (s, 3H); LC-MS: m/z 437.1 (M+H)⁺.

Intermediate-21a.1: ¹H NMR (400 MHz, DMSO-d₆): δ 10.31 (s, 1H), 8.52 (s, 1H), 7.92 (s, 1H), 7.37 (d, J=8.8 Hz, 2H), 7.07 (s, 1H), 6.91 (d, J=8.8 Hz, 2H), 5.58 (s, 2H), 3.85 (s, 3H), 3.71 (s, 3H), 2.25 (s, 3H), 2.06 (s, 3H); LC-MS: m/z 419.2 (M+H)⁺.

### Intermediate-22: 6-(3,5-Dimethylisoxazol-4-yl)-7-methoxy-1-(pyridin-2-ylmethyl) quinolin-2(1H)-one

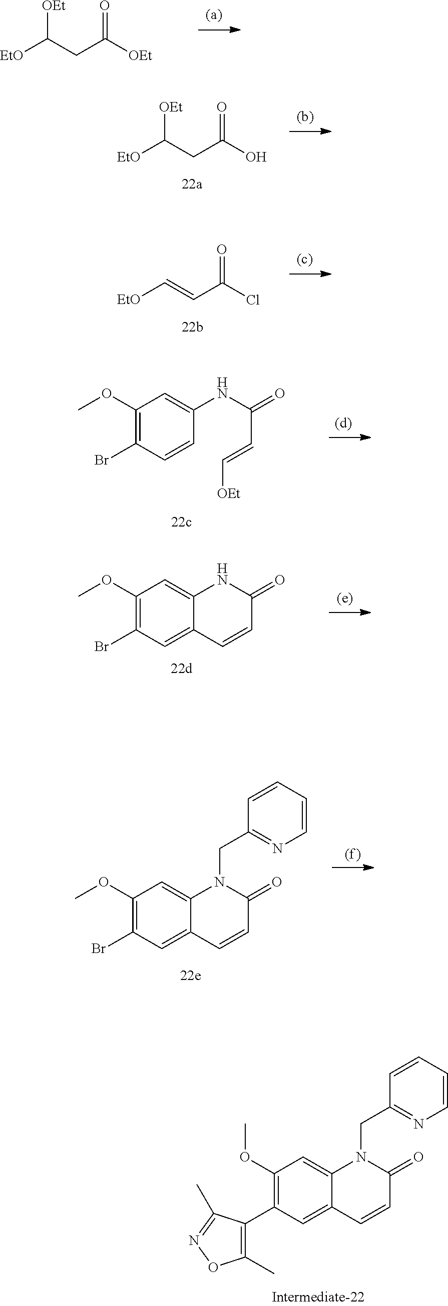

### Step-a: 3, 3-Diethoxypropanoic Acid (22a)

To a suspension of ethyl 3,3-diethoxypropanoate (15.0 g, 78.88 mmol) in water (32 mL) was added NaOH (4.10 g, 102.6 mmol) followed by stirring at 110° C. for 1.5 h. The mixture was cooled, acidified to pH ˜3 with 3 N HCl and extracted with EtOAc (500 ml×2). The organic layer was washed with water (200 mL) and brine (100 mL), dried over sodium sulphate and concentrated under reduced pressure. The residue was used for next step without further purification (11.50 g, 91%). ¹H NMR (400 MHz, DMSO-d₆): δ 12.20 (s, 1H), 4.81 (t, J=5.9 Hz, 1H), 3.58-3.59 (m, 2H), 3.48-3.40 (m, 2H), 2.60-2.40 (m, 2H), 1.09 (t, J=7.3 Hz, 6H).

### Step-b: 3-Ethoxyacryloyl Chloride (22b)

To an ice cooled compound of 3,3-diethoxypropanoic acid (5.00 g, 31.05 mmol) was added thionyl chloride (10.0 mL, 142.9 mmol) over a period of 10 min followed by stirring at 80° C. for 1.5 h. The mixture was concentrated and dried under reduced pressure to afford the title product as a clear dark brown liquid (3.0 g, 73%). ¹H NMR (400 MHz, DMSO-d₆): δ 7.50 (d, J=12.2 Hz, 1H), 5.14 (d, J=12.2 Hz, 1H), 3.94 (q, J=7.3 Hz, 2H), 1.24 (t, J=7.3 Hz, 3H).

### Step-c: (E/Z)—N-(4-bromo-3-methoxyphenyl)-3-ethoxyacrylamide (22c)

To an ice cooled solution of 4-bromo-3-methoxyaniline (3.00 g, 14.85 mmol) in pyridine (20 mL) was added (E/Z)-3-ethoxyacryloyl chloride (2.98 g, 22.27 mmol) over a period of 5 min followed by stirring at RT for 16 h. The mixture was diluted with ice cooled water and extracted with EtOAc (150 ml×2). The combined organic layer was washed with 1N HCl, water (150 mL) and brine (100 mL), dried over sodium sulphate and concentrated under reduced pressure. The residue was used for next step without further purification (3.20 g, 72%). ¹H NMR (400 MHz, DMSO-d₆): δ 9.86 (s, 1H), 7.54-7.42 (m, 3H), 7.12-7.08 (m, 1H), 5.50 (d, J=12.7 Hz, 1H), 3.95 (q, J=6.9 Hz, 2H), 3.80 (s, 3H), 1.27 (t, J=7.3 Hz, 3H); LC-MS: m/z 301.1 (M+H)⁺.

### Step-d: 6-Bromo-7-methoxyquinolin-2(1H)-one (22d)

A solution of (E/Z)—N-(4-bromo-3-methoxyphenyl)-3-ethoxyacrylamide (3.0 g, 10.0 mmol) in concentrated H₂SO₄ (30 mL) was stirred at RT for 2 h. The mixture was poured over crushed ice and the solids were filtered, washed thoroughly with water and vacuum dried. The residue was directly used for the next step without further purification (2.08 g, 82%). ¹H NMR (400 MHz, DMSO-d₆): δ 12.70 (brs, 1H), 7.94 (s, 1H), 7.80 (d, J=9.8 Hz, 1H), 6.92 (s, 1H), 6.36 (d, J=9.8 Hz, 1H), 3.88 (s, 3H); LC-MS: m/z 256.0 (M+H)⁺.

### Step-e: 6-Bromo-7-methoxy-1-(pyridin-2-ylmethyl)quinolin-2(1H)-one (22e)

To a cold solution of 6-bromo-7-methoxyquinolin-2(1H)-one (0.300 g, 1.18 mmol) in dry DMF (5 ml) at 0° C. was added NaH (0.034 g, 1.42 mmol). After 15 min, 2-(bromomethyl)-pyridine hydro bromide (0.36 g, 1.42 mmol) was added and the resulting mixture was stirred at RT for 2 h. The mixture was quenched with ice water and solids were separated, filtered, washed thoroughly with water and dried under reduced pressure to afford the title compound (0.2 g). ¹H NMR (400 MHz, DMSO-d₆): δ 8.49 (d, J=4.4 Hz, 1H), 8.01 (s, 1H), 7.87 (d, J=9.2 Hz, 3H), 7.78-7.77 (m, 1H), 7.29-7.26 (m, 2H), 7.08 (s, 1H), 6.58 (d, J=9.2 Hz, 1H), 5.62 (s, 2H), 3.79 (s, 3H). MS (ES) m/e 347.0 (M+2H)²⁺.

### Step-f: 6-(3,5-Dimethylisoxazol-4-yl)-7-methoxy-1-(pyridin-2-ylmethyl)quinolin-2(1H)-one (Intermediate-22)

To a stirred solution of 6-bromo-7-methoxy-1-(pyridin-2-ylmethyl) quinolin-2(1H)-one (0.200 g, 0.58 mmol) in 10 ml of 1,4 dioxane:water (7:3) mixture was added 3,5-dimethylisoxazol-4-yl) boronic acid (0.164 g, 1.16 mmol) and K₂CO₃ (0.240 g, 1.74 mmol). The resulting mixture was degassed with nitrogen for 15 min and Pd (PPh₃)₄ (0.033 g, 0.029 mmol) was added. The mixture was stirred at 90° C. for 2 h. The mixture was then diluted with DCM and washed with water (50 ml) and dried over Na₂SO₄. Filtration and then concentration in vacuum was followed by chromatography on silica (20% EtOAc in hexanes) to give the title product as a solid (0.142 g, 67%). ¹H NMR (400 MHz, DMSO-d₆): δ 8.52 (d, J=4.4 Hz, 1H), 7.90 (d, J=9.6 Hz, 1H), 7.79-7.75 (m, 1H), 7.62 (s, 1H), 7.33-7.28 (m, 2H), 7.12 (s, 1H), 6.58 (d, J=9.6 Hz, 1H), 5.65 (s, 2H), 3.74 (s, 3H), 2.24 (s, 3H), 2.05 (s, 3H). MS (ES) m/e 362.3 (M+H)⁺.

### Intermediate-23

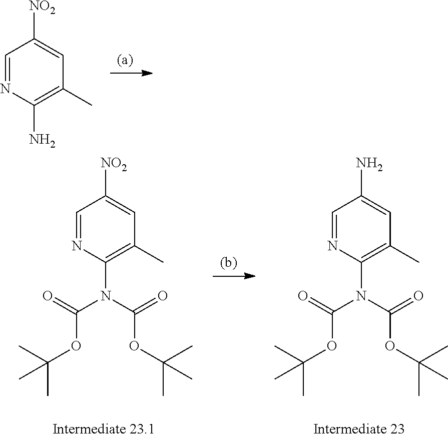 [SMILES: Cc1cc([N+](=O)[O-])cnc1N ; Cc1cc([N+](=O)[O-])cnc1N(C(=O)OC(C)(C)C)C(=O)OC(C)(C)C ; Cc1cc(N)cnc1N(C(=O)OC(C)(C)C)C(=O)OC(C)(C)C]

### Step-a: Intermediate 23.1

To a solution of 3-methyl-5-nitropyridin-2-amine (0.1 g, 0.65 mmol) in DCM (10 mL) was added di tert butyl dicarbonate (0.33 mL, 1.43 mmol) and DMAP (0.016 g, 0.13 mmol) followed by stirring at RT for 2 h. The mixture was concentrated under reduced pressure and purified by combi flash to afford intermediate 21.1 as white solid (0.23 g, 99%). ¹H NMR (400 MHz, DMSO-d₆): δ 9.15 (d, J=2.9 Hz, 1H), 8.67 (d, J=3.0 Hz, 1H), 2.29 (s, 3H), 1.37 (s, 18H).

### Step-b: Intermediate 23

To a solution of Intermediate 21.1 (2.1 g, 5.94 mmol) in MeOH (20 mL) was added 10% Pd—C (0.3 g) followed by stirring under H₂ bladder pressure at RT for 2 h. The mixture was filtered through celite followed by washing with EtOAc. The filtrate was concentrated under reduced pressure to afford the title compound as pale yellow solid (1.5 g). ¹H NMR (400 MHz, DMSO-d₆): δ 7.57 (d, J=2.9 Hz, 1H), 6.81 (d, J=2.4 Hz, 1H), 5.32 (s, 2H), 1.97 (s, 3H), 1.35 (s, 18H); LC-MS: m/z 324.3 (M+H).

### Intermediate-24: tert-Butyl (5-amino-3-methylpyridin-2-yl) carbamate

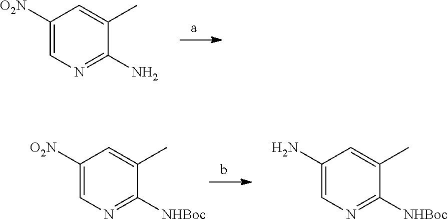 [SMILES: Cc1cc([N+](=O)[O-])cnc1N ; Cc1cc([N+](=O)[O-])cnc1NC(=O)OC(C)(C)C ; Cc1cc(N)cnc1NC(=O)OC(C)(C)C]

### Step-a: tert-Butyl (3-methyl-5-nitropyridin-2-yl) carbamate

To a solution of 3-methyl-5-nitropyridin-2-amine (1.0 g, 6.53 mmol) in DMF (30 mL) was added cesium carbonate (3.2 g, 9.79 mmol) and Boc anhydride (4.5 mL, 19.59 mmol) followed by stirring at RT for 16 h. The mixture was diluted with EtOAc and washed with water. The organic layer was dried over sodium sulphate and concentrated under reduced pressure. The residue was purified by combi-flash to afford the title compound as orange solid (0.7 g, 42%). ¹H NMR (400 MHz, DMSO-d₆): δ 9.71 (s, 1H), 8.99 (d, J=2.4 Hz, 1H), 8.41 (d, J=2.0 Hz, 1H), 2.28 (s, 3H), 1.45 (s, 9H); LC-MS: m/z 252.2 (M−H)⁻.

### Step-b: tert-Butyl (5-amino-3-methylpyridin-2-yl) carbamate

To a solution of tert-butyl (3-methyl-5-nitropyridin-2-yl) carbamate (0.7 g, 2.76 mmol) in MeOH (10 mL) was added 10% Pd—C (0.3 g) followed by stirring under H₂ bladder pressure at RT for 2 h. The mixture was filtered through celite and the bed was washed with EtOAc. The filtrate was concentrated under reduced pressure. The residue was purified by combi-flash to afford the title compound as orange solid (0.5 g, 81%). ¹H NMR (400 MHz, DMSO-d₆): δ 8.51 (s, 1H), 7.57 (d, J=3.0 Hz, 1H), 6.77 (d, J=2.4 Hz, 1H), 5.12 (s, 2H), 2.03 (s, 3H), 1.41 (s, 9H); LC-MS: m/z 224.2 (M+H)⁺.

### Intermediate-25: 5-Amino-1,3-dimethylpyridin-2(1H)-one

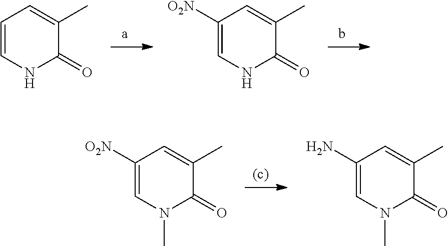

### Step-a: 3-Methyl-5-nitropyridin-2(1H)-one

To a cold solution of 3-methylpyridin-2(1H)-one (2.0 g, 18.34 mmol) in H₂SO₄ (10 mL) was added HNO₃ (1.0 mL) followed by heating to 100° C. for 4 h. The mixture was poured into ice water and extracted with EtOAc. The organic layer was dried over sodium sulphate and concentrated under reduced pressure. The residue was washed with n-pentane to afford the title compound as an off white solid (1.5 g). ¹H NMR (400 MHz, DMSO-d₆): δ 12.54-12.50 (bs, 1H), 8.55 (d, J=3.0 Hz, 1H), 8.05 (dd, J=2.9 Hz & 1.0 Hz, 1H), 2.04 (s, 3H); LC-MS: m/z 155.1 (M+H)⁺.

### Step-b: 1, 3-Dimethyl-5-nitropyridin-2(1H)-one

To a solution of 3-methyl-5-nitropyridin-2(1H)-one (1.5 g, 9.74 mmol) in DMF (10 mL) was added potassium carbonate (4.0 g, 29.22 mmol) and methyl iodide (0.91 mL, 14.61 mmol) followed by stirring at RT for 16 h. The mixture was diluted with EtOAc and washed with water. The organic layer was dried over sodium sulphate and concentrated under reduced pressure. The residue was washed with n-pentane to afford the title compound as an off white solid (1.2 g). ¹H NMR (400 MHz, DMSO-d₆): δ 9.10 (d, J=2.9 Hz, 1H), 8.07 (d, J=1.5 Hz, 1H), 3.57 (s, 3H), 2.08 (s, 3H); LC-MS: m/z 169.1 (M+H)⁺.

### Step-c: 5-Amino-1,3-dimethylpyridin-2(1H)-one

To a solution of 1, 3-dimethyl-5-nitropyridin-2(1H)-one (0.6 g, 3.57 mmol) in mixture of MeOH (5 mL) and THF (5 mL) was added 10% Pd—C (0.3 g) followed by stirring under H₂ bladder pressure at RT for 16 h. The mixture was filtered through celite and the bed was washed with EtOAc. The filtrate was concentrated under reduced pressure to afford the title compound as brown solid (0.5 g). LC-MS: m/z 139.2 (M+H)⁺.

The present invention is further exemplified, but not limited, by the following examples that illustrate the preparation of compounds according to the invention.

### Example-I: 3,6-Bis(3,5-dimethylisoxazol-4-yl)-7-methoxy-1-(pyridin-2-ylmethyl) quinolin-2(1H)-one (Compound-1)

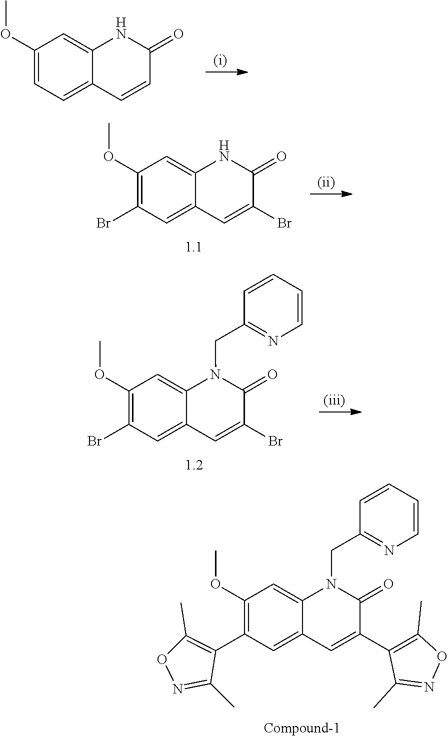

### Step-i: 3,6-dibromo-7-methoxyquinolin-2(1H)-one (1.1)

To a cooled solution of 7-methoxyquinolin-2(1H)-one (2.0 g, 11.36 mmol) in DMF (10 mL) was added N-bromosuccinimide (2.0 g, 11.93 mmol) portion wise followed by stirring at RT for 16 h. The mixture was quenched with ice water, separated, filtered, washed thoroughly with water and dried under reduced pressure to afford the title compound (2.3 g, mixture with mono bromo compound); LC-MS: m/z 334 (M+H)⁺.

### Step-ii: 3,6-Dibromo-7-methoxy-1-(pyridin-2-ylmethyl)quinolin-2(1H)-one (1.2)

The process of this step was adopted from step-e of intermediate-2. ¹H NMR (400 MHz, DMSO-d₆): δ 8.48 (s, 2H), 8.02 (s, 1H), 7.78 (t, J=7.8 Hz, 1H), 7.36 (d, J=7.8 Hz, 1H), 7.31-7.28 (m, 1H), 7.09 (s, 1H), 5.71 (s, 2H), 3.81 (s, 3H); LC-MS: m/z 425.0 (M+H)⁺.

### Step-iii: 3,6-bis (3,5-dimethylisoxazol-4-yl)-7-methoxy-1-(pyridin-2-ylmethyl)-quinolin-2(1H)-one (Compound-1)

The process of this step was adopted from step-e of intermediate-1. The desired di substituted compound was isolated by preparative HPLC from the mixture of mono and di substituted compounds. ¹H NMR (400 MHz, DMSO-d₆): δ 8.54 (d, J=4.4 Hz, 1H), 8.01 (s, 1H), 7.79 (t, J=6.4 Hz, 1H), 7.66 (s, 1H), 7.36 (d, J=7.2 Hz, 1H), 7.33-7.32 (m, 1H), 7.19 (s, 1H), 5.72 (s, 2H), 3.77 (s, 3H), 2.37 (s, 3H), 2.26 (s, 3H), 2.19 (s, 3H), 2.06 (s, 3H); LC-MS: m/z 457.2 (M+H)⁺.

### Example-II: N-(3-(6-(3,5-dimethylisoxazol-4-yl)-7-methoxy-2-oxo-1-(pyridin-2-ylmethyl),1,2-dihydroquinolin-3-yl)phenyl)acetamide (Compound-2)

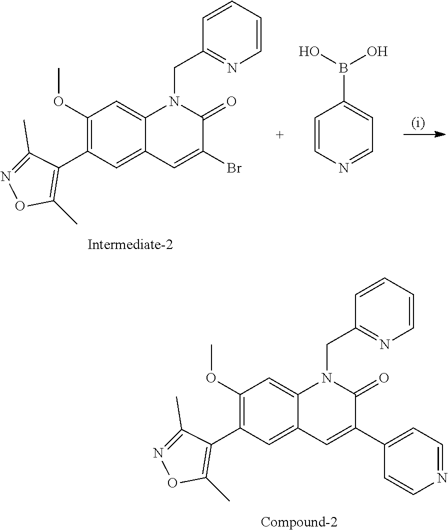 [SMILES: COc1cc2c(cc1-c1c(C)noc1C)cc(Br)c(=O)n2Cc1ccccn1 ; OB(O)c1ccncc1 ; COc1cc2c(cc1-c1c(C)noc1C)cc(-c1ccncc1)c(=O)n2Cc1ccccn1]

To a solution of 3-bromo-6-(3,5-dimethylisoxazol-4-yl)-7-methoxy-1-(pyridin-2-ylmethyl)quinolin-2(1H)-one (intermediate-2) (0.11 g, 0.23 mmol) in 1,2-DME (3 mL) and H₂O (1 mL) were added pyridin-4-ylboronic acid (0.09 g, 0.69 mmol) and sodium carbonate (0.06 g, 0.57 mmol) followed by degassing with nitrogen purging for 20 min. Then tetrakistriphenylphosphine palladium (0.03 g, 0.023 mmol) was added and the mixture was heated at 90° C. for 16 h. The mixture was then diluted with EtOAc (50 mL), washed with water (50 mL) and brine (50 mL), dried over sodium sulphate and concentrated under reduced pressure. The residue was purified by preparative TLC to afford the title compound as a pale green solid (0.03 g, 30%); ¹H NMR (400 MHz, DMSO-d₆) δ 8.65-8.63 (m, 2H), 8.53 (d, J=4.4 Hz, 1H), 7.36 (s, 1H), 7.83-7.77 (m, 3H), 7.73 (s, 1H), 7.41 (d, J=8.0 Hz, 1H), 7.32-7.29 (m, 1H), 7.71 (s, 1H), 5.75 (s, 2H), 3.78 (s, 3H), 2.27 (s, 3H), 2.08 (s, 3H); LC-MS: m/z 439.2 (M+H)+.

The below compounds were prepared by a procedure similar to the one described in Example-II by using appropriate bromo compounds and reacting with suitable boronic acids or esters in presence of suitable palladium catalyst and reagents in the presence of suitable solvents at appropriate reaction conditions. The physiochemical characteristics of the compounds are also summarized.

| | | | Characterization Data |
|---|---|---|---|
| | | | ¹H NMR (400 MHz, DMSO-d₆)/LC- |
| No | Reactant | Obtained Compound | MS: |
| 3 | 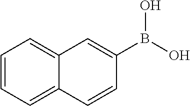 [SMILES: OB(O)c1ccc2ccccc2c1] | 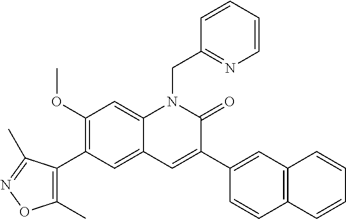 [SMILES: COc1cc2c(cc1-c1c(C)noc1C)cc(-c1ccc3ccccc3c1)c(=O)n2Cc1ccccn1] | δ 8.61 (d, J = 3.9 Hz, 1H), 8.41 (s, 1H), 8.35 (s, 1H), 8.04-7.96 (m, 4H), 7.86 (t, J = 7.8 Hz, 1H), 7.81 (s, 1H), 7.60 (m, 2H), 7.47 (d, J = 7.8 Hz, 1H), 7.39- 7.38 (m, 1H), 7.24 (s, 1H), 5.84 (s, 2H), 3.84 (s, 3H), 2.34 (s, 3H), 2.15 (s, 3H); LC-MS: m/z 488.2 (M + H)⁺. |
| 4 | 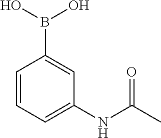 | 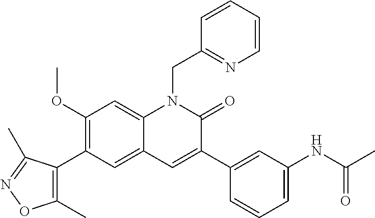 [SMILES: COc1cc2c(cc1-c1c(C)noc1C)cc(-c1cccc(NC(C)=O)c1)c(=O)n2Cc1ccccn1] | δ 10.08 (s, 1H), 8.54 (d, J = 4.4 Hz, 1H), 8.09 (s, 1H), 7.91 (s, 1H), 7.79- 7.78 (m, 1H), 7.71 (s, 1H), 7.68-7.66 (m, 1H), 7.39-7.29 (m, 4H), 7.14 (s, 1H), 5.74 (s, 2H), 3.77 (s, 3H), 2.27 (s, 3H), 2.08 (s, 3H), 2.05 (s, 3H); LC- MS: m/z 495.2 (M + H)⁺. |
| 5 | 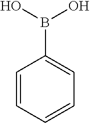 | 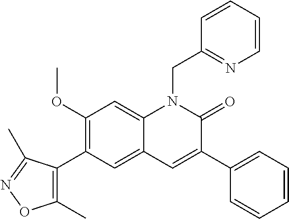 [SMILES: COc1cc2c(cc1-c1c(C)noc1C)cc(-c1ccccc1)c(=O)n2Cc1ccccn1] | δ 8.54 (d, J = 4.4 Hz, 1H), 8.13 (s, 1H), 7.79-7.74 (m, 3H), 7.70 (s, 1H), 7.46-7.42 (m, 2H), 7.38 (d, J = 7.8 Hz, 2H), 7.32-7.29 (m, 1H), 7.15 (s, 1H), 5.74 (s, 2H), 3.77 (s, 3H), 2.27 (s, 3H), 2.08 (s, 3H); LC-MS: m/z 438.2 (M + H)⁺. |
| 6 | 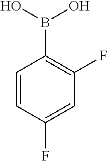 [SMILES: OB(O)c1ccc(F)cc1F] | 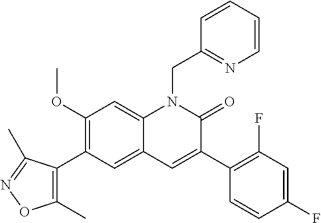 [SMILES: COc1cc2c(cc1-c1c(C)noc1C)cc(-c1ccc(F)cc1F)c(=O)n2Cc1ccccn1] | δ 8.54 (d, J = 4.4 Hz, 1H), 8.05 (s, 1H), 7.81-7.76 (m, 1H), 7.68 (s, 1H), 7.64- 7.58 (m, 1H), 7.37-7.30 (m, 3H), 7.21- 7.16 (m, 2H), 5.72 (s, 2H), 3.78 (s, 3H), 2.26 (s, 3H), 2.07 (s, 3H); LC- MS: m/z 474.1 (M + H)⁺. |
| 7 | 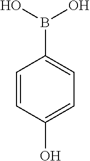 | 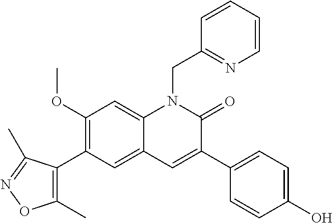 [SMILES: COc1cc2c(cc1-c1c(C)noc1C)cc(-c1ccc(O)cc1)c(=O)n2Cc1ccccn1] | δ 9.57 (s, 1H), 8.53 (d, J = 4.4 Hz. 1H), 8.02 (s, 1H), 7.78 (t, J = 7.3 Hz, 1H), 7.65 (s, 1H), 7.61 (d, J = 8.3 Hz, 2H), 7.36 (d, J = 7.8 Hz, 1H), 7.32-7.28 (m, 1H), 7.13 (s, 1H), 7.62 (d, J = 8.3 Hz, 1H), 5.72 (s, 2H), 3.76 (s, 3H), 2.26 (s, 3H), 2.07 (s, 3H); LC-MS: m/z 452.2 (M − H)⁻. |
| 8 | 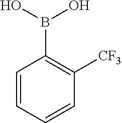 [SMILES: OB(O)c1ccccc1C(F)(F)F] | 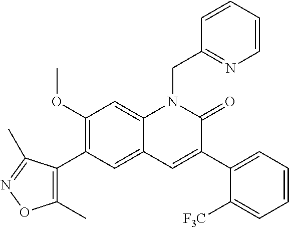 [SMILES: COc1cc2c(cc1-c1c(C)noc1C)cc(-c1ccccc1C(F)(F)F)c(=O)n2Cc1ccccn1] | δ 8.56 (d, J = 4.4 Hz, 1H), 7.88 (s, 1H), 7.84-7.72 (m, 3H), 7.67 (s, 1H), 7.66- 7.62 (m, 1H), 7.50 (d, J = 7.3 Hz, 1H), 7.33-7.26 (m, 2H), 7.20 (s, 1H), 5.71- 5.67 (m, 2H), 3.78 (s, 3H), 2.25 (s, 3H), 2.06 (s, 3H); LC-MS: m/z 506.1 (M + H)⁺. |
| 9 | 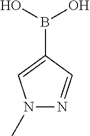 | 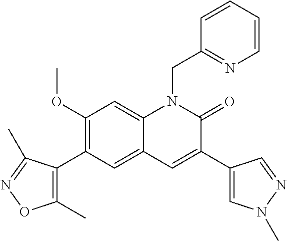 [SMILES: COc1cc2c(cc1-c1c(C)noc1C)cc(-c1cnn(C)c1)c(=O)n2Cc1ccccn1] | δ 8.60 (d, J = 4.4 Hz, 1H), 8.40 (s, 1H), 7.97 (s, 1H), 7.93 (s, 1H), 7.63-7.61 (m, 1H), 7.35 (d, J = 7.8 Hz, 1H), 7.31 (s, 1H), 7.23 (d, J = 5.9 Hz, 1H), 7.20 (s, 1H), 5.97 (bs, 2H), 3.97 (s, 3H), 3.80 (s, 3H), 2.29 (s, 3H), 2.14 (s, 3H); LC-MS: m/z 442.2 (M + H)⁺. |
| 10 | 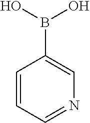 | 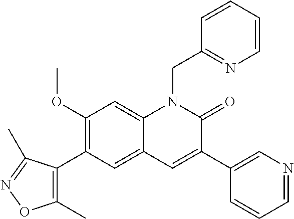 [SMILES: COc1cc2c(cc1-c1c(C)noc1C)cc(-c1cccnc1)c(=O)n2Cc1ccccn1] | δ 8.92 (d, J = 1.5 Hz, 1H), 8.54 (dd, J₁ = 3.4 Hz, J₂ = 11.2 Hz, 2H), 8.26 (s, 1H), 8.16 (d, J = 7.8 Hz, 1H), 7.79- 7.76 (m, 1H), 7.70 (s, 1H), 7.49-7.46 (m, 1H), 7.40 (d, J = 7.8 Hz, 1H), 7.32- 7.30 (m, 1H) 7.16 (s, 1H), 5.75 (s, 2H), 3.77 (s, 3H), 2.27 (s, 3H), 2.07 (s, 3H); LC-MS: m/z 439.2 (M + H)⁺. |
| 11 | 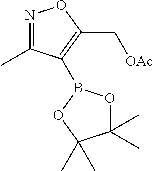 | 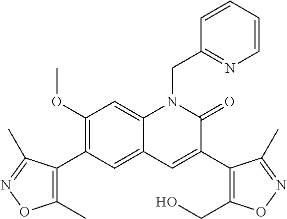 [SMILES: COc1cc2c(cc1-c1c(C)noc1C)cc(-c1c(C)noc1CO)c(=O)n2Cc1ccccn1] | δ 8.55-8.52 (m, 1H), 8.02 (s, 1H), 7.81-7.76 (m, 1H), 7.65 (s, 1H), 7.36 (d, J = 7.8 Hz, 1H), 7.33-7.29 (m, 1H), 7.19 (s, 1H), 5.72 (s, 2H), 5.52 (t, J = 5.9 Hz, 1H), 4.51 (d, J = 5.9 Hz, 2H), 3.78 (s, 3H), 2.26 (s, 3H), 2.22 (s, 3H), 2.06 (s, 3H); LC-MS: m/z 473.2 (M + H)⁺. |
| 12 | 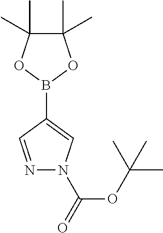 | 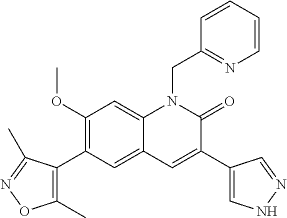 [SMILES: COc1cc2c(cc1-c1c(C)noc1C)cc(-c1cn[nH]c1)c(=O)n2Cc1ccccn1] | δ 12.95 (bs, 1H), 8.53 (d, J = 4.3 Hz, 1H), 8.53-8.10 (m, 3H), 7.79-7.53 (m, 1H), 7.59 (s, 1H), 7.36-7.29 (m, 2H), 7.13 (s, 1H), 5.76 (s, 2H), 3.75 (s, 3H), 2.27 (s, 3H), 2.08 (s, 3H); LC- MS: m/z 428.2 (M + H)⁺. |
| 13 | 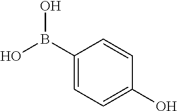 | 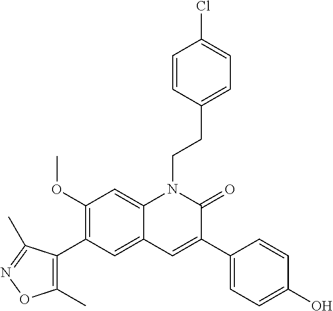 [SMILES: COc1cc2c(cc1-c1c(C)noc1C)cc(-c1ccc(O)cc1)c(=O)n2CCc1ccc(Cl)cc1] | ¹H NMR (400 MHz, DMSO-d₆): δ 9.54 (s, 1H), 7.96 (s, 1H), 7.63 (s, 1H), 7.55 (d, J = 8.8 Hz, 2H), 7.37- 7.32 (m, 4H), 7.00 (s, 1H), 6.80 (d, J = 8.3 Hz, 2H), 4.58 (t, J = 6.8 Hz, 2H), 3.93 (s, 3H), 3.02 (t, J = 7.3 Hz, 2H), 2.28 (s, 3H), 2.10 (s, 3H); LCMS: m/z 501.2 (M + H)⁺. |
| 14 | 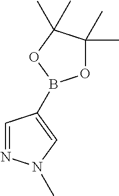 | 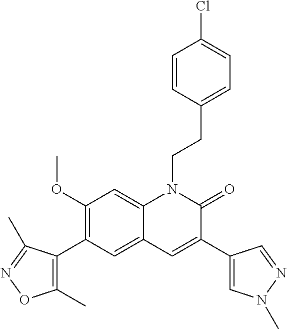 [SMILES: COc1cc2c(cc1-c1c(C)noc1C)cc(-c1cnn(C)c1)c(=O)n2CCc1ccc(Cl)cc1] | ¹H NMR (400 MHz, DMSO-d₆): δ 8.41 (s, 1H), 8.23 (s, 1H),8.02 (s, 1H), 7.57 (s, 1H), 7.36 (s, 4H), 7.00 (s, 1H), 4.61 (t, J = 7.3 Hz, 2H), 3.93 (s, 3H), 3.89 (s, 3H), 3.02 (t, J = 7.3 Hz, 2H), 2.29 (s, 3H), 2.09 (s, 3H); LCMS: m/z 489.2 (M + H)⁺. |

### Example-III: 6-(3,5-dimethylisoxazol-4-yl)-7-methoxy-3-(1H-pyrazol-1-yl)-1-(pyridin-2-ylmethyl)quinolin-2(1H)-one (Compound-15)

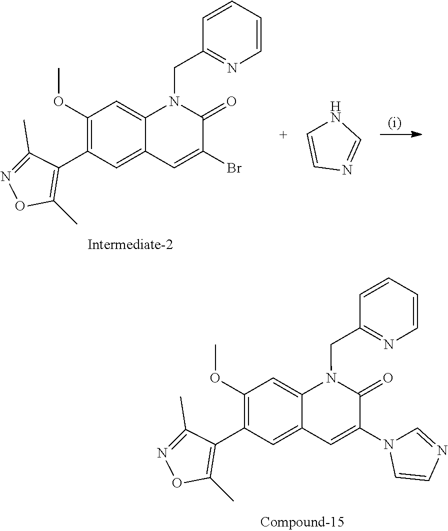 [SMILES: COc1cc2c(cc1-c1c(C)noc1C)cc(Br)c(=O)n2Cc1ccccn1 ; c1c[nH]cn1 ; COc1cc2c(cc1-c1c(C)noc1C)cc(-n1ccnc1)c(=O)n2Cc1ccccn1]

To a solution of 3-bromo-6-(3,5-dimethylisoxazol-4-yl)-7-methoxy-1-(pyridin-2-ylmethyl)quinolin-2(1H)-one (intermediate-2) (0.1 g, 0.22 mmol) in DMSO (3 mL) in a sealed tube were added imidazole (0.03 g, 0.45 mmol), L-proline (0.005 g, 0.04 mmol), copper (I) iodide (0.009 g, 0.04 mmol) and potassium carbonate (0.1 g, 0.68 mmol) followed by heating at 110° C. for 16 h. The mixture was diluted with EtOAc (50 mL), washed with water (50 mL) and brine (50 mL), dried over sodium sulphate and concentrated under reduced pressure. The residue was purified by preparative HPLC to afford the title compound as an off white solid (0.02 g, 20%). ¹H NMR (400 MHz, DMSO-d₆): δ 8.53 (d, J=4.4 Hz, 1H), 8.12 (s, 1H), 7.82-7.80 (m, 1H), 7.68 (s, 1H), 7.43 (d, J=7.8 Hz, 1H), 7.33-7.32 (m, 1H), 7.27 (s, 1H), 7.20 (s, 1H), 7.08 (s, 1H), 6.73 (s, 1H), 5.73 (s, 2H), 3.78 (s, 3H), 2.21 (s, 3H), 2.0 (s, 3H); LC-MS: m/z 428.2 (M+H)+.

The below compounds were prepared by a procedure similar to the one described in Example-III by using appropriate bromo compounds reacting with suitable reactants in the presence of suitable reagents, catalysts and solvents at appropriate reaction conditions. The physiochemical characteristics of the compounds are also summarized.

| | | | Characterization Data |
|---|---|---|---|
| | | | ¹H NMR (400 MHz, DMSO-d₆)/LC- |
| No | Reactant | Obtained Compound | MS: |
| 16 | 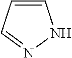 | 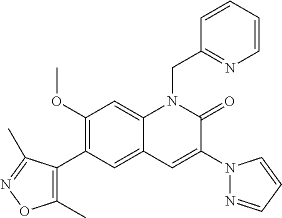 [SMILES: COc1cc2c(cc1-c1c(C)noc1C)cc(-n1cccn1)c(=O)n2Cc1ccccn1] | δ 8.54 (d, J = 3.4 Hz, 1H), 8.40 (d, J = 1.9 Hz, 1H), 7.92 (s, 1H), 7.85 (s, 1H), 7.80 (t, J = 6.9 Hz, 1H), 7.42 (d, J = 7.9 Hz, 1H), 7.33-7.30 (m, 1H), 7.22 (s, 1H), 6.75 (s, 1H), 6.63 (s, 1H), 5.73 (s, 2H), 3.78 (s, 3H), 2.22 (s, 3H), 2.03 (s, 3H); LC-MS: m/z 428.1 (M + H)⁺. |
| 17 | 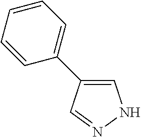 [SMILES: c1ccc(-c2cn[nH]c2)cc1] | 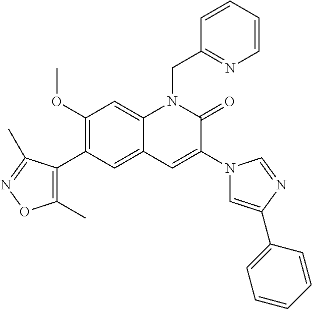 [SMILES: COc1cc2c(cc1-c1c(C)noc1C)cc(-n1cnc(-c3ccccc3)c1)c(=O)n2Cc1ccccn1] | ¹H NMR (400 MHz, CD₃OD): δ 8.53 (d, J = 4.4 Hz, 1H), 8.35 (s, 1H), 8.25 (s, 1H), 7.98 (s, 1H), 7.80-7.78 (m, 3H), 7.64 (s, 1H), 7.47 (d, J = 7.9 Hz, 1H), 7.41-7.34 (m, 3H), 7.34-7.26 (m, 1H), 7.14 (s, 1H), 5.86 (s, 2H), 3.80 (s, 3H), 2.28 (s, 3H), 2.11 (s, 3H); LC- MS: m/z 504.2 (M + H)⁺. |
| 18 | 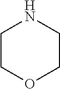 | 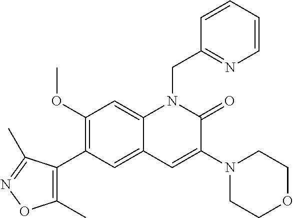 [SMILES: COc1cc2c(cc1-c1c(C)noc1C)cc(N1CCOCC1)c(=O)n2Cc1ccccn1] | δ 8.52 (d, J = 4.4 Hz, 1H), 7.74 (t, J = 7.3 Hz, 1H), 7.52 (s, 1H), 7.30-7.27 (m, 2H), 7.23 (s, 1H), 7.08 (s, 1H), 5.68 (s, 2H), 3.76 (m, 4H), 3.71 (s, 3H), 3.14 (m, 4H), 2.24 (s, 3H), 2.05 (s, 3H); LC-MS: m/z 447.4 (M + H)⁺. |
| 19 | 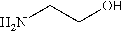 | 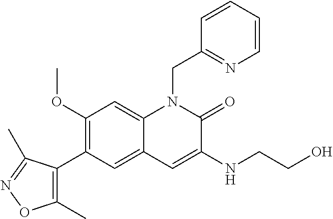 [SMILES: COc1cc2c(cc1-c1c(C)noc1C)cc(NCCO)c(=O)n2Cc1ccccn1] | δ 8.52 (d, J = 4.0 Hz, 1H), 7.77 (t, J = 4.4 Hz, 1H), 7.36 (s, 1H), 7.30 (d, J = 7.2 Hz, 2H), 7.05 (s, 1H), 6.68 (s, 1H), 5.72 (s, 2H), 5.59 (t, J = 5.6 Hz, 1H), 4.87 (t, J = 5.2 Hz, 1H), 3.67 (s, 3H), 3.66-3.64 (m, 2H), 3.18 (q, J = 5.9 Hz, 2H), 2.24 (s, 3H), 2.05 (s, 3H); LC-MS: m/z 421.2 (M + H)⁺. |
| 20 | 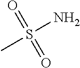 | 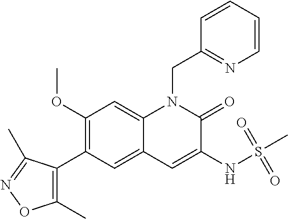 [SMILES: COc1cc2c(cc1-c1c(C)noc1C)cc(NS(C)(=O)=O)c(=O)n2Cc1ccccn1] | δ 9.03 (bs, 1H), 8.53 (bs, 1H), 7.85 (s, 1H), 7.79 (t, J = 7.2 Hz, 1H), 7.67 (s, 1H), 7.38 (d, J = 7.2 Hz, 1H), 7.32- 7.30 (m, 1H), 7.12 (s, 1H), 5.73 (s, 2H), 3.74 (s, 3H), 3.14 (s, 3H), 2.25 (s, 3H), 2.06 (s, 3H); LC-MS: m/z 455.1 (M + H)⁺. |
| 21 | 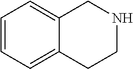 | 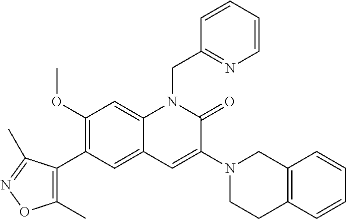 [SMILES: COc1cc2c(cc1-c1c(C)noc1C)cc(N1CCc3ccccc3C1)c(=O)n2Cc1ccccn1] | δ 8.53 (d, J = 4.4 Hz, 1H), 7.79-7.75 (m, 1H), 7.53 (s, 1H), 7.33-7.28 (m, 3H), 7.18 (s, 4H), 7.09 (s, 1H), 5.71 (s, 2H), 4.33 (s, 2H), 3.71 (s, 3H), 3.51 (t, J = 5.9 Hz, 2H), 2.97 (t, J = 5.8 Hz, 2H), 2.25 (s, 3H), 2.06 (s, 3H); LC-MS: m/z 493.2 (M + H)⁺. |
| 22 | 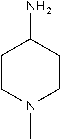 | 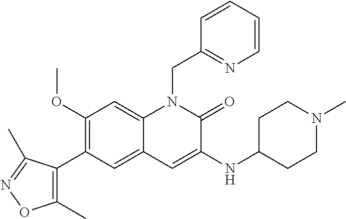 [SMILES: COc1cc2c(cc1-c1c(C)noc1C)cc(NC1CCN(C)CC1)c(=O)n2Cc1ccccn1] | δ 8.52 (d, J = 4.9 Hz, 1H), 7.79-7.75 (m, 1H), 7.38 (s, 1H), 7.31-7.27 (m, 2H), 7.05 (s, 1H), 6.72 (s, 1H), 5.71 (s, 2H), 5.28 (d, J = 7.9 Hz, 1H), 3.67 (s, 3H), 3.30-3.20 (m, 1H), 2.76-2.71 (m, 2H), 2.24 (s, 3H), 2.19 (s, 3H), 2.10-2.05 (m, 2H), 2.05 (s, 3H), 1.97- 1.93 (m, 2H), 1.56-1.51 (m, 2H); LC- MS: m/z 474.3 (M + H)⁺. |
| 23 | 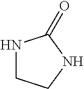 | 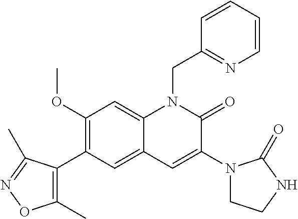 [SMILES: COc1cc2c(cc1-c1c(C)noc1C)cc(N1CCNC1=O)c(=O)n2Cc1ccccn1] | δ 8.53 (d, J = 4.4 Hz, 1H), 7.96 (s, 1H). 7.81-7.76 (m, 1H), 7.63 (s, 1H), 7.35- 7.30 (m, 2H), 7.12 (s, 1H), 6.86 (s, 1H), 5.69 (s, 2H), 3.93 (t, J = 7.4 Hz, 2H), 3.74 (s, 3H), 3.42-3.36 (m, 2H), 2.25 (s, 3H), 2.06 (s, 3H); LC-MS: m/z 446.2 (M + H)⁺. |
| 24 | 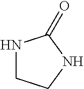 | 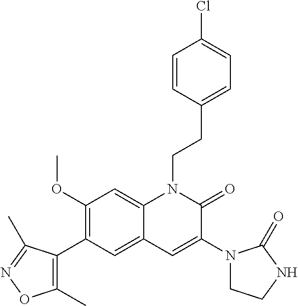 [SMILES: COc1cc2c(cc1-c1c(C)noc1C)cc(N1CCNC1=O)c(=O)n2CCc1ccc(Cl)cc1] | ¹H NMR (400 MHz, DMSO-d₆): δ 7.90 (s, 1H), 7.61 (s, 1H), 7.36-7.30 (m, 4H), 7.00 (s, 1H), 6.83 (s, 1H), 4.55 (t, J = 7.3 Hz, 2H), 3.92 (s, 3H), 3.86 (t, J = 7.4 Hz, 2H), 3.39 (t, J = 7.8 Hz, 2H), 2.99 (t, J = 7.3 Hz, 2H), 2.27 (s, 3H), 2.08 (s, 3H); LCMS: m/z 493.2 (M + H)⁺. |

### Example-IV: 6-(3,5-Dimethylisoxazol-4-yl)-3-(4-hydroxypiperidine-1-carbonyl)-7-methoxy-1-(pyridin-2-ylmethyl)quinolin-2(1H)-one (Compound-25)

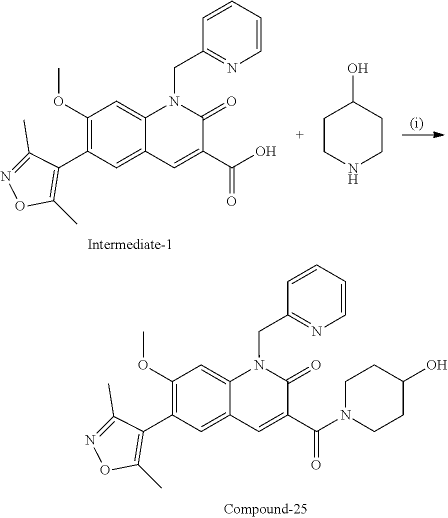 [SMILES: COc1cc2c(cc1-c1c(C)noc1C)cc(C(=O)O)c(=O)n2Cc1ccccn1 ; OC1CCNCC1 ; COc1cc2c(cc1-c1c(C)noc1C)cc(C(=O)N1CCC(O)CC1)c(=O)n2Cc1ccccn1]

To a solution of 6-(3,5-dimethylisoxazol-4-yl)-7-methoxy-2-oxo-1-(pyridin-2-yl-methyl)-1,2-dihydroquinoline-3-carboxylicacid (intermediate-1) (0.1 g, 0.25 mmol) in DMF (5 mL) were added piperidin-4-ol (0.04 g, 0.37 mmol), HOBt (0.1 g, 0.74 mmol), EDC.HCl (0.14 g, 0.74 mmol) and triethylamine (0.1 mL, 0.74 mmol) followed by stirring at RT for 4 h. The mixture was diluted with EtOAc (50 mL), washed with water (50 mL) and brine (50 mL), dried over sodium sulphate and concentrated under reduced pressure. The residue was purified by preparative TLC to afford the title compound as an off white solid (0.025 g, 20%); ¹H NMR (400 MHz, DMSO-d₆): δ 8.51 (d, J=3.9 Hz, 1H), 7.97 (s, 1H), 7.79 (t, J=7.3 Hz, 1H), 7.62 (s, 1H), 7.36-7.29 (m, 2H), 7.16 (s, 1H), 5.68 (s, 2H), 4.76 (s, 1H), 4.1-4.0 (m, 1H), 3.77 (s, 3H), 3.44-3.09 (m, 4H), 2.25 (s, 3H), 2.05 (s, 3H), 1.76-1.70 (m, 2H), 1.38-1.35 (m, 2H); LC-MS: m/z 489.2 (M+H)⁺.

The below compounds were prepared by a procedure similar to the one described in Example-IV by using intermediate-1 or compound-77 prepared according to Example-XII as starting compounds and reacting with suitable reactants in the presence of suitable reagents and solvents at appropriate reaction conditions. The physiochemical characteristics of the compounds are also summarized.

| | | | Characterization Data |
|---|---|---|---|
| | | | ¹H NMR (400 MHz, DMSO-d₆)/LC- |
| No | Reactant | Obtained Compound | MS |
| 26 | 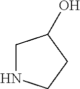 | 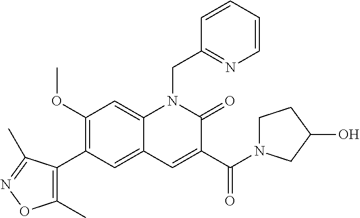 [SMILES: COc1cc2c(cc1-c1c(C)noc1C)cc(C(=O)N1CCC(O)C1)c(=O)n2Cc1ccccn1] | δ 8.52 (d, J = 4.4 Hz, 1H), 8.03 (d, J = 8.8 Hz, 1H), 7.79 (t, J = 6.4 Hz, 1H), 7.70 (d, J = 3.4 Hz, 1H), 7.36-7.29 (m, 2H), 7.16 (s, 1H), 5.70 (s, 2H), 4.97(dd, J₁ = 2.9 Hz, J₂ = 19, 1H), 4.34-4.25 (m, 1H), 3.77 (s, 3H), 3.54-3.51 (m, 4H), 2.25 (s, 3H), 2.06 (s, 3H), 2.01-1.79 (m, 2H); LC-MS: m/z 475.2 (M + H)⁺. |
| 27 | 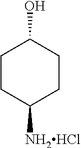 | 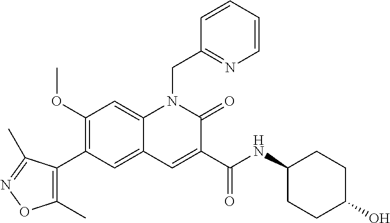 [SMILES: COc1cc2c(cc1-c1c(C)noc1C)cc(C(=O)N[C@H]1CC[C@H](O)CC1)c(=O)n2Cc1ccccn1] | δ 9.61 (d, J = 7.8 Hz, 1H),8.88(s, 1H), 8.50 (d, J = 4.4 Hz, 1H), 7.96 (s, 1H), 7.81-7.77 (m, 1H), 7.41 (d, J = 7.8 Hz, 1H), 7.32-7.29 (m, 1H), 7.16 (s, 1H), 5.76 (s, 2H), 4.57 (d, J = 3.9 Hz, 1H), 3.79 (s, 3H), 3.80 (m, 1H), 3.46-3.45 (m, 1H), 2.27 (s, 3H), 2.08 (s,3H), 1.92-1.82 (m, 4H), 1.35-1.23 (m, 4H); LC-MS: m/z 503.2 (M + H)⁺. |
| 28 | 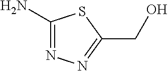 | 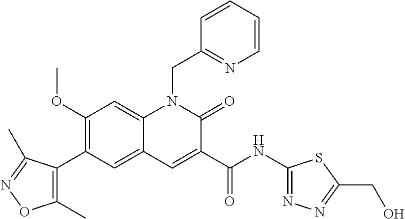 [SMILES: COc1cc2c(cc1-c1c(C)noc1C)cc(C(=O)Nc1nnc(CO)s1)c(=O)n2Cc1ccccn1] | δ 13.44 (s, 1H), 9.12 (s, 1H), 8.51 (d, J = 4.9 Hz, 1H), 8.07 (s, 1H), 7.82- 7.80 (m, 1H), 7.52 (d, J = 7.8 Hz, 1H), 7.34-7.31 (m, 1H), 7.29 (s, 1H), 6.09 (t, J = 6.3 Hz, 1H), 5.86 (s, 2H), 4.81 (d, J = 5.8 Hz, 2H), 3.85 (s, 3H), 2.29 (s, 3H), 2.10 (s, 3H); LC- MS: m/z 519.2 (M + H)⁺. |
| 29 | 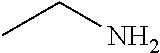 | 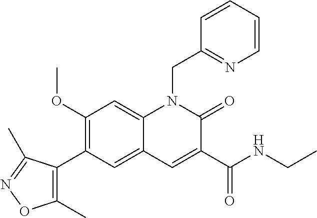 [SMILES: CCNC(=O)c1cc2cc(-c3c(C)noc3C)c(OC)cc2n(Cc2ccccn2)c1=O] | δ 9.62 (t, J = 5.3 Hz, 1H), 8.88 (s, 1H), 8.50 (d, J = 4.9 Hz, 1H), 7.96 (s, 1H), 7.81-7.77 (m, 1H), 7.41 (d, J = 7.8 Hz, 1H), 7.32-7.29 (m, 1H), 7.19 (s, 1H), 5.76 (s, 2H), 3.80 (s, 3H), 3.39-3.35 (m, 2H), 2.27 (s, 3H), 2.08 (s, 3H), 1.55 (t, J = 7.3 Hz, 3H); LC-MS: m/z 433.1 (M + H)⁺. |
| 30 | 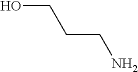 | 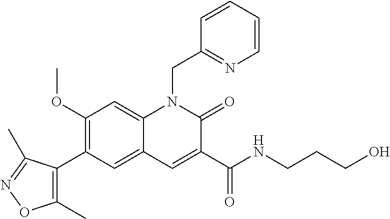 [SMILES: COc1cc2c(cc1-c1c(C)noc1C)cc(C(=O)NCCCO)c(=O)n2Cc1ccccn1] | δ 9.69 (t, J = 5.3 Hz, 1H), 8.88 (s, 1H), 8.50 (d, J = 4.9 Hz, 1H), 7.96 (s, 1H), 7.81-7.77 (m, 1H), 7.41 (d, J = 7.8 Hz, 1H), 7.32-7.29 (m, 1H), 7.18 (s, 1H), 5.77 (s, 2H), 4.55 (t, J = 5.4 Hz, 1H), 3.80 (s, 3H), 3.51- 3.46 (m, 2H), 3.43-3.38 (m, 2H), 2.27 (s, 3H), 2.08 (s, 3H), 1.72-1.65 (m, 2H); LC-MS: m/z 463.2 (M + H)⁺. |
| 31 | 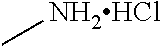 | 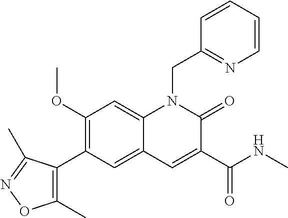 [SMILES: CNC(=O)c1cc2cc(-c3c(C)noc3C)c(OC)cc2n(Cc2ccccn2)c1=O] | δ 9.52 (d, J = 4.4 Hz, 1H), 8.88 (s, 1H), 8.50 (d, J = 4.4 Hz, 1H), 7.96 (s, 1H), 7.81-7.76 (m, 1H), 7.40 (d, J = 7.8 Hz, 1H), 7.32-7.29 (m, 1H), 7.19 (s, 1H), 5.76 (s, 2H), 3.80 (s, 3H), 2.89 (d, J = 4.4 Hz, 3H), 2.27 (s, 3H), 2.08 (s, 3H); LC-MS: m/z 419.2 (M + H)⁺. |
| 32 | 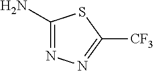 | 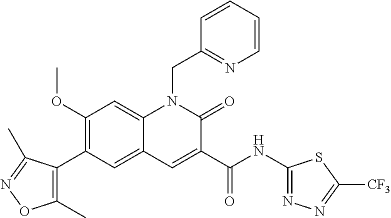 [SMILES: COc1cc2c(cc1-c1c(C)noc1C)cc(C(=O)Nc1nnc(C(F)(F)F)s1)c(=O)n2Cc1ccccn1] | δ 13.95 (s, 1H), 9.15 (s, 1H), 8.52 (d, J = 4.2 Hz, 1H), 8.01 (s, 1H), 7.84- 7.81 (m, 1H), 7.54 (d, J = 7.8 Hz, 1H), 7.35-7.33 (m, 1H), 7.31 (s, 1H), 5.87 (s, 2H), 3.86 (s, 3H), 2.29 (s, 3H), 2.10 (s, 3H); LC-MS: m/z 557.0 (M + H)⁺. |
| 33 | 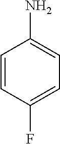 | 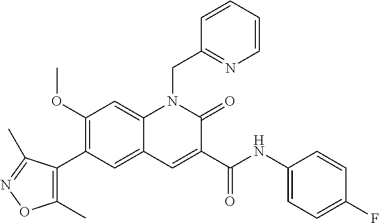 [SMILES: COc1cc2c(cc1-c1c(C)noc1C)cc(C(=O)Nc1ccc(F)cc1)c(=O)n2Cc1ccccn1] | δ 11.99 (s, 1H), 9.03 (s, 1H), 8.52 (d, J = 4.4 Hz, 1H), 8.03 (s, 1H), 7.84- 7.76 (m, 3H), 7.48 (d, J = 7.8 Hz, 1H), 7.34-7.30 (m, 1H), 7.25-7.19 (m, 3H), 5.84 (s, 2H), 3.82 (s, 3H), 2.29 (s, 3H), 2.10 (s, 3H); LC-MS: m/z 499.2 (M + H)⁺. |
| 34 | 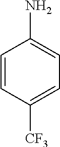 [SMILES: Nc1ccc(C(F)(F)F)cc1] | 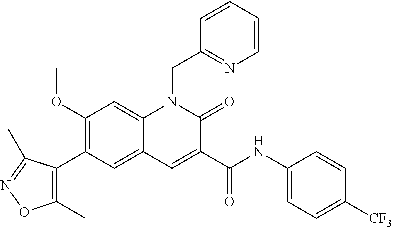 [SMILES: COc1cc2c(cc1-c1c(C)noc1C)cc(C(=O)Nc1ccc(C(F)(F)F)cc1)c(=O)n2Cc1ccccn1] | δ 12.29 (s, 1H), 9.05 (s, 1H), 8.51 (d, J = 4.9 Hz, 1H), 8.05 (s, 1H), 7.97 (d, J = 8.3 Hz, 2H), 7.83-7.81 (m, 1H), 7.75 (d, J = 8.8 Hz, 2H), 7.49 (d, J = 8.3 Hz, 1H), 7.34-7.31 (m, 1H), 7.23 (s, 1H), 5.85 (s, 2H), 3.82 (s, 3H), 2.29 (s, 3H), 2.09 (s, 3H); LC-MS: m/z 549.2 (M + H)⁺. |
| 35 | 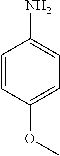 | 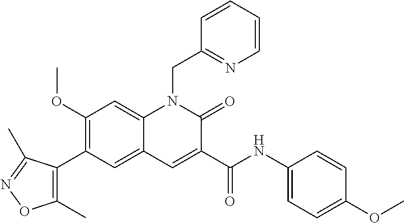 [SMILES: COc1ccc(NC(=O)c2cc3cc(-c4c(C)noc4C)c(OC)cc3n(Cc3ccccn3)c2=O)cc1] | δ 11.85 (s, 1H), 9.01 (s, 1H), 8.52 (d, J = 4.4 Hz, 1H), 8.02 (s, 1H), 7.83- 7.79 (m, 1H), 7.68 (d, J = 8.8 Hz, 2H), 7.47 (d, J = 8.3 Hz, 1H), 7.34- 7.30 (m, 1H), 7.22 (s, 1H), 6.95 (d, J = 9.3 Hz, 2H), 5.84 (s, 2H), 3.82 (s, 3H), 3.76 (s, 3H), 2.29 (s, 3H), 2.10 (s, 3H); LC-MS: m/z 511.2 (M + H)⁺. |
| 36 | 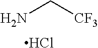 | 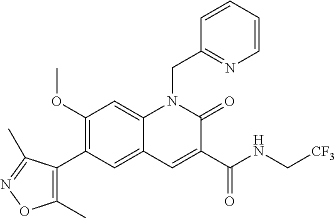 [SMILES: COc1cc2c(cc1-c1c(C)noc1C)cc(C(=O)NCC(F)(F)F)c(=O)n2Cc1ccccn1] | δ 10.11 (t, J = 6.4 Hz, 1H), 8.96 (s, 1H), 8.51 (d, J = 4.4 Hz, 1H), 8.00 (s, 1H), 7.82-7.78 (m, 1H), 7.44 (d, J = 7.9 Hz, 1H), 7.33-7.26 (m, 1H), 7.21 (s, 1H), 5.79 (s, 2H), 4.29-4.21 (m, 2H), 3.82 (s, 3H), 2.28 (s, 3H), 2.09 (s, 3H); LC-MS: m/z 487.1 (M + H)⁺. |
| 37 | 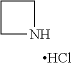 | 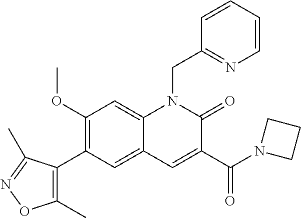 [SMILES: COc1cc2c(cc1-c1c(C)noc1C)cc(C(=O)N1CCC1)c(=O)n2Cc1ccccn1] | δ 8.52 (d, J = 4.4 Hz, 1H), 8.14 (s, 1H), 7.80-7.76 (m, 1H), 7.73 (s, 1H), 7.35-7.30 (m, 2H), 7.16 (s, 1H), 5.69 (s, 2H),4.08-3.99 (m, 4H), 3.77 (s, 3H), 2.25 (s, 3H), 2.25-2.21 (m, 2H), 2.06 (s, 3H); LC-MS: m/z 445.2 (M + H)⁺. |
| 38 | 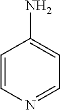 | 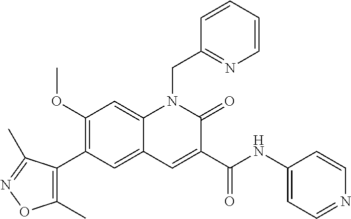 [SMILES: COc1cc2c(cc1-c1c(C)noc1C)cc(C(=O)Nc1ccncc1)c(=O)n2Cc1ccccn1] | δ 12.24 (s, 1H), 9.05 (s, 1H), 8.52- 8.50 (m, 3H), 8.05 (s, 1H), 7.83-7.79 (m, 1H), 7.76 (d, J = 5.9 Hz, 2H), 7.49 (d, J = 7.8 Hz, 1H), 7.33-7.30 (m, 1H), 7.23 (s, 1H), 5.85 (s, 2H), 3.83 (s, 3H), 2.29 (s, 3H), 2.10 (s, 3H); LC-MS: m/z 482.2 (M + H)⁺. |
| 39 | 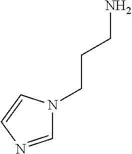 | 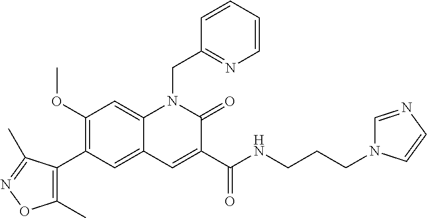 [SMILES: COc1cc2c(cc1-c1c(C)noc1C)cc(C(=O)NCCCn1ccnc1)c(=O)n2Cc1ccccn1] | δ 9.69 (t, J = 5.8 Hz, 1H), 8.88 (s, 1H), 8.51 (d, J = 4.9 Hz, 1H), 7.96 (s, 1H), 7.80 (t, J = 7.4 Hz, 1H), 7.68 (s, 1H), 7.42 (d, J = 7.8 Hz, 1H), 7.32- 7.41 (m, 1H), 7.23 (s, 1H), 7.19 (s, 1H), 6.90 (s, 1H), 5.77 (s, 2H), 4.03 (t, J = 6.9 Hz, 2H), 3.80 (s, 3H), 3.34- 3.33 (m, 2H), 2.27 (s, 3H), 2.08 (s, 3H), 2.00 (t, J = 7.3 Hz, 2H); LC-MS: m/z 513.2 (M + H)⁺. |
| 40 | 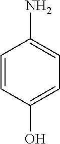 | 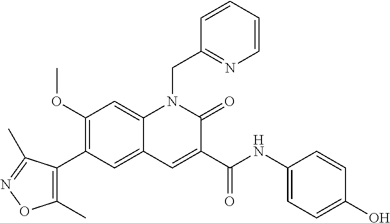 [SMILES: COc1cc2c(cc1-c1c(C)noc1C)cc(C(=O)Nc1ccc(O)cc1)c(=O)n2Cc1ccccn1] | δ 11.76 (s, 1H), 9.32 (s, 1H), 9.00 (s, 1H), 8.52 (d, J = 4.9 Hz, 1H), 8.01 (s, 1H), 7.83-7.78 (m, 1H), 7.54 (d, J = 8.8 Hz, 2H), 7.47 (d, J = 7.8 Hz, 1H), 7.33-7.30 (m, 1H), 7.22 (s, 1H), 6.77 (d, J = 8.8 Hz, 2H), 5.83 (s, 2H), 3.82 (s, 3H), 2.28 (s, 3H), 2.09 (s, 3H); LC-MS: m/z 497.2 (M + H)⁺. |
| 41 | 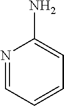 | 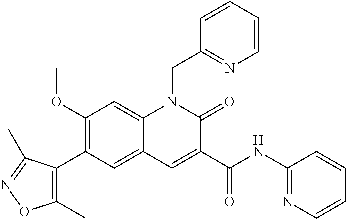 [SMILES: COc1cc2c(cc1-c1c(C)noc1C)cc(C(=O)Nc1ccccn1)c(=O)n2Cc1ccccn1] | δ 12.40 (s, 1H), 9.07 (s, 1H), 8.52 (d, J = 4.9 Hz, 1H), 8.36-8.31 (m, 2H), 8.04 (s, 1H), 7.89-7.79 (m, 2H), 7.50 (d, J = 8.3 Hz, 1H), 7.34-7.31 (m, 1H), 7.27 (m, 1H), 7.18-7.15 (m, 1H), 5.83 (s, 2H), 3.84 (s, 3H), 2.29 (s, 3H), 2.10 (s, 3H); LC-MS: m/z 482.2 (M + H)⁺. |
| 42 | 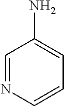 | 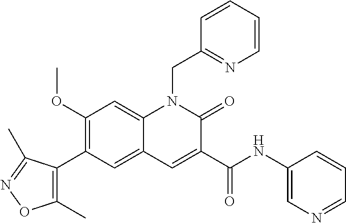 [SMILES: COc1cc2c(cc1-c1c(C)noc1C)cc(C(=O)Nc1cccnc1)c(=O)n2Cc1ccccn1] | ¹δ 12.06 (s, 1H), 9.05 (s, 1H), 8.90 (d, J = 2.0 Hz, 1H), 8.51 (d, J = 4.4 Hz, 1H), 8.34 (d, J = 4.9 Hz, 1H), 8.33- 8.23 (m, 1H), 8.04 (s, 1H), 7.84-7.79 (m, 1H), 7.49 (d, J = 7.8 Hz, 1H), 7.44-7.41 (m, 1H), 7.34-7.23 (m, 1H), 7.23 (s, 1H), 5.85 (s, 2H), 3.82 (s, 3H), 2.29 (s, 3H), 2.10 (s, 3H); LC-MS: m/z 482.2 (M + H)⁺. |
| 43 | 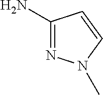 | 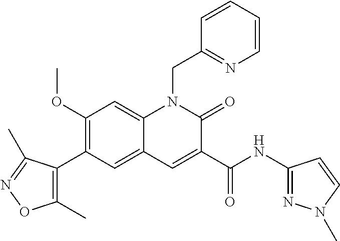 [SMILES: COc1cc2c(cc1-c1c(C)noc1C)cc(C(=O)Nc1ccn(C)n1)c(=O)n2Cc1ccccn1] | δ 12.1 (s, 1H), 9.0 (s, 1H), 8.52 (d, J = 4.4 Hz, 1H), 8.0 (s, 1H), 7.81 (t, J = 7.8 Hz, 1H), 7.63 (s, 1H), 7.48 (d, J = 7.8 Hz, 1H), 7.33-7.30 (m, 1H), 7.25 (s, 1H), 6.63 (d, J = 1.9 Hz, 1H), 5.82 (s, 2H), 3.82 (s, 3H), 3.72 (s, 3H), 2.28 (s, 3H), 2.09 (s, 3H); LC- MS: m/z 485.2 (M + H)⁺. |
| 44 | 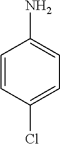 | 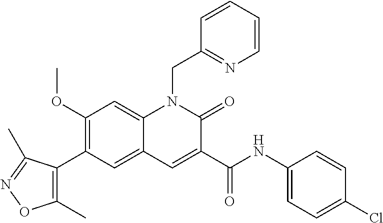 [SMILES: COc1cc2c(cc1-c1c(C)noc1C)cc(C(=O)Nc1ccc(Cl)cc1)c(=O)n2Cc1ccccn1] | δ 12.07 (s, 1H), 9.03 (s, 1H), 8.51 (d, J = 4.4 Hz, 1H), 8.03 (s, 1H), 7.80 (d, J = 8.8 Hz, 3H), 7.47 (d, J = 7.8 Hz, 1H), 7.43 (d, J = 8.8 Hz, 2H), 7.32 (t, J = 6.9 Hz, 1H), 7.22 (s, 1H), 5.84 (s, 2H), 3.82 (s, 3H), 2.28 (s, 3H), 2.09 (s, 3H); LC-MS: m/z 515.2 (M + H)⁺. |
| 45 | 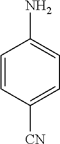 | 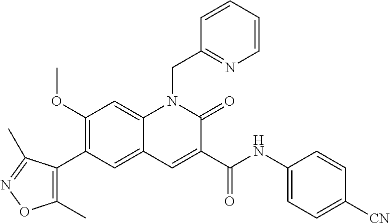 [SMILES: COc1cc2c(cc1-c1c(C)noc1C)cc(C(=O)Nc1ccc(C#N)cc1)c(=O)n2Cc1ccccn1] | δ 12.33 (s, 1H), 9.05 (s, 1H), 8.51 (d, J = 4.9 Hz, 1H), 8.05 (s, 1H), 7.97- 7.95 (m, 2H), 7.85-7.81 (m, 3H), 7.48 (d, J = 7.8 Hz, 1H), 7.34-7.32 (m, 1H), 7.22 (s, 1H), 5.84 (s, 2H), 3.82 (s, 3H), 2.29 (s, 3H), 2.10 (s, 3H); LC-MS: m/z 506.2 (M + H)⁺. |
| 46 | 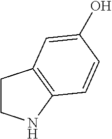 | 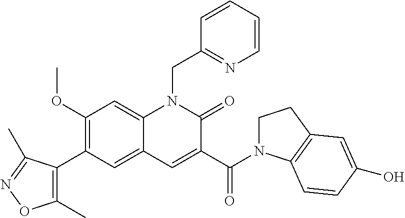 [SMILES: COc1cc2c(cc1-c1c(C)noc1C)cc(C(=O)N1CCc3cc(O)ccc31)c(=O)n2Cc1ccccn1] | δ 9.28 (s, 1H), 8.53 (d, J = 4.4 Hz, 1H), 8.13 (s, 1H), 7.95 (d, J = 8.8 Hz, 1H), 7.85-7.78 (m, 1H), 7.72 (s, 1H), 7.38 (d, J = 7.8 Hz, 1H), 7.32-7.30 (m, 1H), 7.17 (s, 1H), 6.68 (s, 1H), 6.65-6.00 (m, 1H), 5.73 (s, 2H), 3.96-3.80 (m, 2H), 3.78 (s, 3H), 3.03 (t, J = 8.3 Hz, 2H), 2.26 (s, 3H), 2.07 (s, 3H); LC-MS: m/z 523.2 (M + H)⁺. |
| 47 | 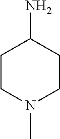 | 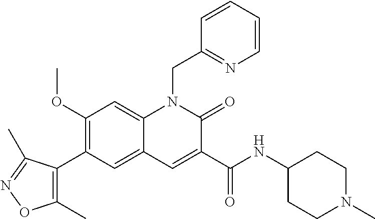 [SMILES: COc1cc2c(cc1-c1c(C)noc1C)cc(C(=O)NC1CCN(C)CC1)c(=O)n2Cc1ccccn1] | δ 9.70 (d, J = 7.8 Hz, 1H), 8.89 (s, 1H), 8.51 (d, J = 4.9 Hz, 1H), 7.97 (s, 1H), 7.81-7.79 (m, 1H), 7.43 (d, J = 7.8 Hz, 1H), 7.33-7.29 (m, 1H), 7.19 (s, 1H), 5.78 (s, 2H), 3.92-3.85 (m, 1H), 3.79 (s, 3H), 2.85-2.75 (m, 2H), 2.35-2.31 (m, 2H), 2.30 (s, 3H), 2.27 (s, 3H), 2.09 (s, 3H), 1.95-1.92 (m, 2H), 1.60-1.54 (m, 2H); LC-MS: m/z 502.3 (M + H)⁺. |
| 48 | 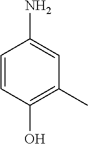 | 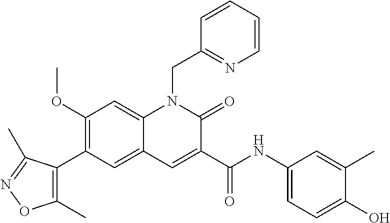 [SMILES: COc1cc2c(cc1-c1c(C)noc1C)cc(C(=O)Nc1ccc(O)c(C)c1)c(=O)n2Cc1ccccn1] | δ 11.73 (s, 1H), 9.20 (s, 1H), 8.98 (s, 1H), 8.52 (d, J = 4.4 Hz, 1H), 8.01 (s, 1H), 7.82-7.77 (m, 1H), 7.46 (d, J = 7.8 Hz, 1H), 7.42-7.40 (m, 2H), 7.34-7.31 (m, 1H), 7.22 (s, 1H), 6.77-6.75 (m, 1H), 5.83 (s, 2H), 3.82 (s, 3H), 2.29 (s, 3H), 2.13 (s, 3H), 2.10 (s, 3H); LC-MS: m/z 511.2 (M + H)⁺. |
| 49 | 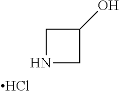 | 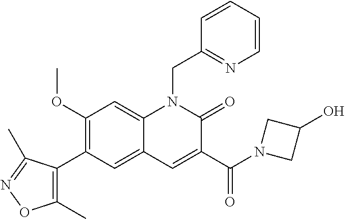 [SMILES: COc1cc2c(cc1-c1c(C)noc1C)cc(C(=O)N1CC(O)C1)c(=O)n2Cc1ccccn1] | δ 8.52 (d, J = 4.2 Hz, 1H), 8.15 (s, 1H), 7.81-7.78 (m, 1H), 7.74 (s, 1H), 7.35-7.48 (m, 2H), 7.16 (s, 1H), 5.75-5.70 (m, 1H), 5.69 (s, 2H), 4.52-4.48 (m, 1H), 4.22-4.16 (m, 2H), 3.88-3.82 (m, 1H), 3.78 (s, 3H), 3.78-3.70 (m, 1H), 2.25 (s, 3H), 2.06 (s, 3H); LC-MS: m/z 461.2 (M + H)⁺. |
| 50 | 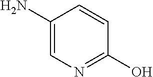 | 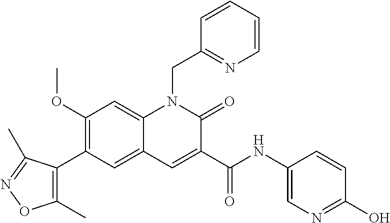 [SMILES: COc1cc2c(cc1-c1c(C)noc1C)cc(C(=O)Nc1ccc(O)nc1)c(=O)n2Cc1ccccn1] | δ 11.55 (s, 1H), 11.50 (bs, 1H), 8.97 (s, 1H), 8.51 (d, J = 4.4 Hz, 1H), 8.11 (d, J = 2.5 Hz, 1H), 8.02 (s, 1H), 7.82- 7.78 (m, 1H), 7.61 (dd, J = 9.2 Hz, J = 2.4 Hz, 1H), 7.46 (d, J = 7.9 Hz, 1H), 7.33-7.30 (m, 1H), 7.21 (s, 1H), 6.38 (d, J = 9.3 Hz, 1H), 5.82 (s, 2H), 3.82 (s, 3H), 2.28 (s, 3H), 2.09 (s, 3H); LC-MS: m/z 498.3 (M + H)⁺. |
| 51* | 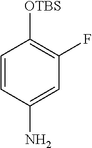 [SMILES: CC(C)(C)[Si](C)(C)Oc1ccc(N)cc1F] | 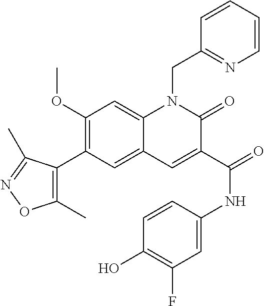 [SMILES: COc1cc2c(cc1-c1c(C)noc1C)cc(C(=O)Nc1ccc(O)c(F)c1)c(=O)n2Cc1ccccn1] | δ 11.86 (s, 1H), 9.71 (s, 1H), 9.0 (s, 1H), 8.51 (d, J = 4.8 Hz, 1H), 8.02 (s, 1H), 7.83-7.76 (m, 2H), 7.46 (d, J = 7.8 Hz, 1H), 7.33-7.30 (m, 1H), 7.22 (s, 1H), 7.20-7.19 (m, 1H), 6.94 (t, J = 9.3 Hz, 1H), 5.83 (s, 2H), 3.81 (s, 3H), 2.28 (s, 3H), 2.09 (s, 3H); LC- MS: m/z 515.2 (M + H)⁺. |
| 52* | 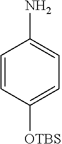 [SMILES: CC(C)(C)[Si](C)(C)Oc1ccc(N)cc1] | 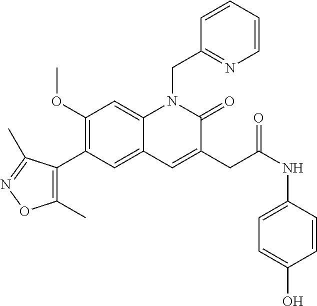 [SMILES: COc1cc2c(cc1-c1c(C)noc1C)cc(CC(=O)Nc1ccc(O)cc1)c(=O)n2Cc1ccccn1] | δ 9.86 (s, 1H), 9.18 (bs, 1H), 8.53 (d, J = 3.9 Hz, 1H), 7.87 (s, 1H), 7.80- 7.78 (m, 1H), 7.62 (s, 1H), 7.38 (d, J = 8.8 Hz, 2H), 7.30 (d, J = 7.4 Hz, 2H), 7.12 (s, 1H), 6.68 (d, J = 8.8 Hz, 2H), 5.68 (s, 2H), 3.74 (s, 3H), 3.60 (s, 2H), 2.25 (s, 3H), 2.06 (s, 3H); LC-MS: m/z 511.2 (M + H)⁺. |
| 53* | 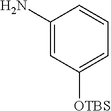 [SMILES: CC(C)(C)[Si](C)(C)Oc1cccc(N)c1] | 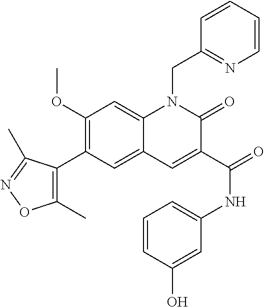 [SMILES: COc1cc2c(cc1-c1c(C)noc1C)cc(C(=O)Nc1cccc(O)c1)c(=O)n2Cc1ccccn1] | δ 11.92 (s, 1H), 9.50 (s, 1H), 9.01 (s, 1H), 8.51 (d, J = 4.4 Hz, 1H), 8.02 (s, 1H), 7.81 (t, J = 7.3 Hz, 1H), 7.48 (d, J = 7.8 Hz, 1H), 7.37 (s, 1H), 7.32 (t, J = 6.3 Hz, 1H), 7.22 (s, 1H), 7.14 (t, J = 8.3 Hz, 1H), 7.01 (d, J = 7.8 Hz, 1H), 6.53 (d, J = 8.3 Hz, 1H), 5.83 (s, 2H), 3.81 (s, 3H), 2.28 (s, 3H), 2.10 (s, 3H); LC-MS: m/z 497.2 (M + H)⁺. |
| 54* | 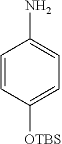 [SMILES: CC(C)(C)[Si](C)(C)Oc1ccc(N)cc1] | 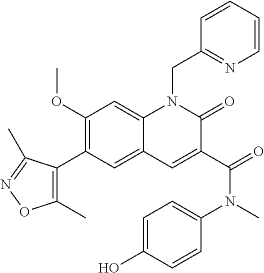 [SMILES: COc1cc2c(cc1-c1c(C)noc1C)cc(C(=O)N(C)c1ccc(O)cc1)c(=O)n2Cc1ccccn1] | δ 8.53 (d, J = 4.4 Hz, 1H), 7.77 (s, 1H), 7.65-7.61 (m, 1H), 7.22-7.15 (m, 4H), 7.06 (s, 1H), 6.76 (d, J = 7.3 Hz, 1H), 6.71 (d, J = 8.8 Hz, 2H), 5.62 (bs, 1H), 5.51 (bs, 2H), 3.77 (s, 3H), 3.49 (s, 3H), 2.26 (s, 3H), 2.11 (s, 3H); LC-MS: m/z 511.2 (M + H)⁺. |
| 55* | 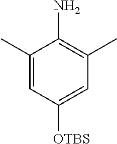 | 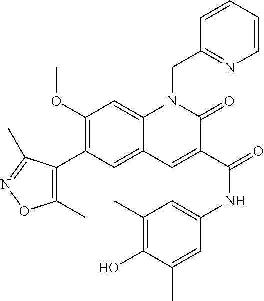 [SMILES: COc1cc2c(cc1-c1c(C)noc1C)cc(C(=O)Nc1cc(C)c(O)c(C)c1)c(=O)n2Cc1ccccn1] | δ 11.72 (s, 1H), 8.97 (s, 1H), 8.52 (d, J = 4.4 Hz, 1H), 8.12 (s, 1H), 8.01 (s, 1H), 7.83-7.78 (m, 1H), 7.47 (d, J = 7.8 Hz, 1H), 7.34-7.31 (m, 3H), 7.28 (s, 1H), 5.82 (s, 2H), 3.82 (s, 3H), 2.29 (s, 3H), 2.17 (s, 6H), 1.99 (s, 3H); LC-MS: m/z 525.2 (M + H)⁺. |
| 56 | 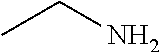 | 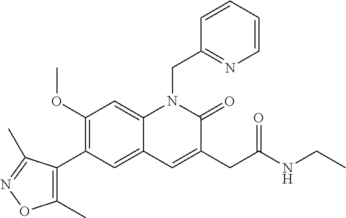 [SMILES: CCNC(=O)Cc1cc2cc(-c3c(C)noc3C)c(OC)cc2n(Cc2ccccn2)c1=O] | δ 8.53 (d, J = 4.4 Hz, 1H), 7.94-7.92 (m, 1H), 7.81 (s, 1H), 7.79-7.75 (m, 1H), 7.59 (s, 1H), 7.31-7.27 (m, 2H), 7.11 (s, 1H), 5.66 (s, 2H), 3.74 (s, 3H), 3.39 (s, 2H), 3.12-3.06 (m, 2H), 2.25 (s, 3H), 2.05 (s, 3H), 1.03 (t, J = 7.4 Hz, 3H); LC-MS: m/z 447.2 (M + H)⁺. |
| 57 | 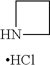 | 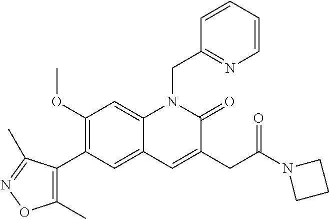 [SMILES: COc1cc2c(cc1-c1c(C)noc1C)cc(CC(=O)N1CCC1)c(=O)n2Cc1ccccn1] | δ 8.51 (d, J = 3.9 Hz, 1H), 7.80-7.74 (m, 2H), 7.59 (s, 1H), 7.30-7.26 (m, 2H), 7.09 (s, 1H), 5.65 (s, 2H), 4.25 (t, J = 7.4 Hz, 2H), 3.85 (t, J = 7.3 Hz, 2H), 3.72 (s, 3H), 3.35 (s, 2H), 2.23 (s, 3H), 2.23-2.18 (m, 2H), 2.04 (s, 3H); LC-MS: m/z 459.2 (M + H)⁺. |
| 58 | NH₄Cl | 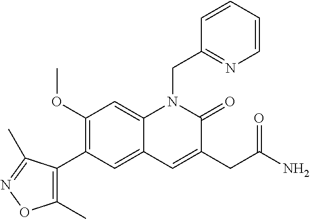 [SMILES: COc1cc2c(cc1-c1c(C)noc1C)cc(CC(N)=O)c(=O)n2Cc1ccccn1] | δ 8.52 (d, J = 4.4 Hz, 1H), 7.80 (s, 1H), 1.11-1.14 (m, 1H), 7.58 (s, 1H), 7.39 (s, 1H), 7.30-7.28 (m, 2H), 7.10 (s, 1H), 6.8 (s, 1H), 5.65 (s, 2H), 3.72 (s, 3H), 3.37 (s, 2H), 2.23 (s, 3H), 2.04 (s, 3H); LC-MS: m/z 419.1 (M + H)⁺. |
| 59 | 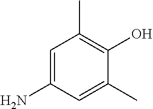 | 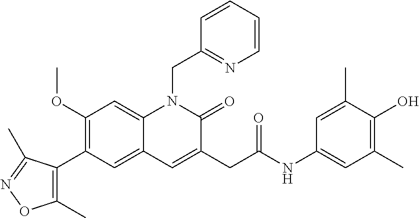 [SMILES: COc1cc2c(cc1-c1c(C)noc1C)cc(CC(=O)Nc1cc(C)c(O)c(C)c1)c(=O)n2Cc1ccccn1] | δ 9.72 (s, 1H), 8.52 (d, J = 4.0 Hz. 1H), 7.95 (s, 1H), 7.86 (s, 1H), 7.77 (t, J = 7.4 Hz, 1H), 7.61 (s, 1H), 7.30 (d, J = 8.4 Hz, 2H), 7.14 (s, 2H), 7.12 (s, 1H), 5.67 (s, 2H), 3.74 (s, 3H), 3.58 (s, 2H), 2.24 (s, 3H), 2.11 (s, 6H), 2.05 (s, 3H); LC-MS: m/z 539.2 (M + H)⁺. |
| 60 | 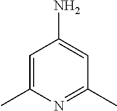 | 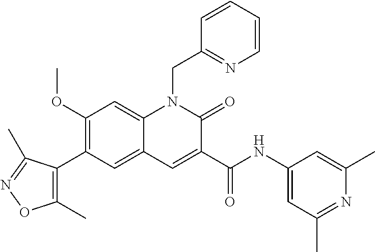 [SMILES: COc1cc2c(cc1-c1c(C)noc1C)cc(C(=O)Nc1cc(C)nc(C)c1)c(=O)n2Cc1ccccn1] | δ 12.45 (bs, 1H), 8.95 (s, 1H), 8.61 (d, J = 4.9 Hz, 1H), 7.73-7.68 (m, 1H), 7.55-7.52 (m, 3H), 7.43 (d, J = 7.8 Hz, 1H), 7.38 (s, 1H), 7.29- 7.26 (m, 1H), 5.78 (s, 2H), 3.89 (s, 3H), 2.63 (s, 6H), 2.29 (s, 3H), 2.14 (s, 3H); LC-MS: m/z 510.1 (M + H)⁺. |
| 61 | 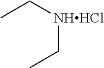 | 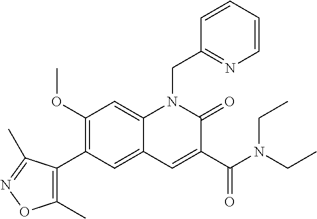 [SMILES: CCN(CC)C(=O)c1cc2cc(-c3c(C)noc3C)c(OC)cc2n(Cc2ccccn2)c1=O] | δ 8.51 (d, J = 4.9 Hz, 1H), 7.94 (s, 1H), 7.79 (t, J = 7.8 Hz, 1H), 7.68 (s, 1H), 7.35 (d, J = 7.8 Hz, 1H), 7.32- 7.29 (m, 1H), 7.17 (s, 1H), 5.68 (s, 2H), 3.77 (s, 3H), 3.44-3.42 (m, 2H), 3.24-3.18 (m, 2H), 2.25 (s, 3H), 2.06 (s, 3H), 1.14 (t, J = 7.3 Hz, 3H), 1.05 (t, J = 7.3 Hz, 3H); LC-MS: m/z 461.2 (M + H)⁺. |
| 62 | 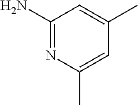 | 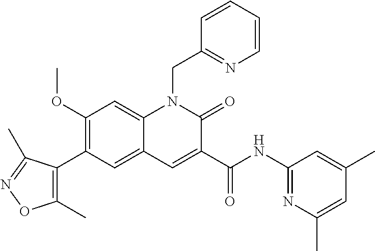 [SMILES: COc1cc2c(cc1-c1c(C)noc1C)cc(C(=O)Nc1cc(C)cc(C)n1)c(=O)n2Cc1ccccn1] | δ 12.23 (s, 1H), 9.01 (s, 1H), 8.49 (d, J = 4.0 Hz, 1H), 8.01 (s, 1H), 7.95 (s, 1H), 7.81-7.77 (m, 1H), 7.47 (d, J = 7.8 Hz, 1H), 7.31-7.28 (m, 1H), 7.25 (s, 1H), 6.84 (s, 1H), 5.80 (s, 2H), 3.81 (s, 3H), 2.35 (s, 3H), 2.30 (s, 3H), 2.26 (s, 3H), 2.07 (s, 3H); LCMS: m/z 510.3 (M + H)⁺. |
| 63 | 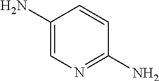 | 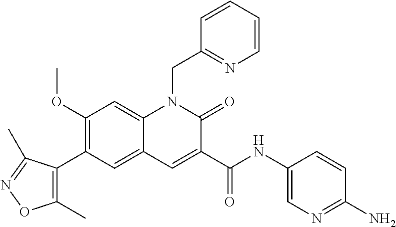 [SMILES: COc1cc2c(cc1-c1c(C)noc1C)cc(C(=O)Nc1ccc(N)nc1)c(=O)n2Cc1ccccn1] | δ 11.61 (s, 1H), 8.98 (s, 1H), 8.51 (d, J = 3.9 Hz, 1H), 8.27 (s, 1H), 8.00 (s, 1H), 7.80-7.77 (m, 2H), 7.46 (d, J = 7.8 Hz, 1H), 7.32 (t, J = 5.4 Hz, 1H), 7.21 (s, 1H), 6.50 (d, J = 8.8 Hz, 1H), 5.93 (bs, 2H), 5.82 (s, 2H), 3.81 (s, 3H), 2.28 (s, 3H), 2.09 (s, 3H); LCMS: m/z 497.2 (M + H)⁺. |
| 64** | 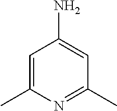 | 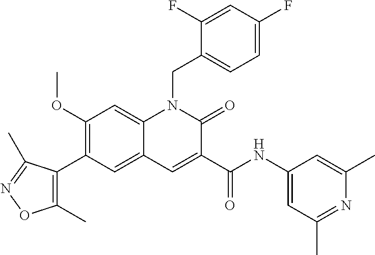 [SMILES: COc1cc2c(cc1-c1c(C)noc1C)cc(C(=O)Nc1cc(C)nc(C)c1)c(=O)n2Cc1ccc(F)cc1F] | δ 12.10 (bs, 1H), 9.03 (s, 1H), 8.08 (s, 1H), 7.46 (bs, 2H), 7.39-7.33 (m, 1H), 7.24-7.18 (m, 1H), 7.09 (s, 1H), 7.04-6.99 (m, 1H), 5.74 (s, 2H), 3.87 (s, 3H), 2.42 (s, 6H), 2.29 (s, 3H), 2.10 (s, 3H); LC-MS: m/z 545.2 (M + H)⁺. |
| 65** | 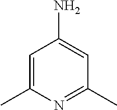 | 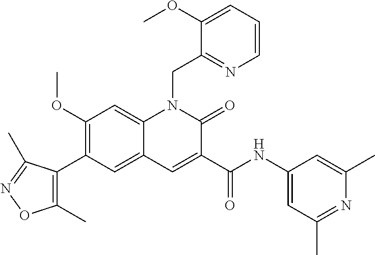 [SMILES: COc1cc2c(cc1-c1c(C)noc1C)cc(C(=O)Nc1cc(C)nc(C)c1)c(=O)n2Cc1ncccc1OC] | ¹H NMR (400 MHz, DMSO-d₆): δ 12.18 (bs, 1H), 9.02 (s, 1H), 8.03 (s, 1H), 7.92 (d, J = 3.9 Hz, 1H), 7.52 (d, J = 7.8 Hz, 1H), 7.43 (bs, 2H), 7.36- 7.28 (m, 1H), 7.00 (s, 1H), 5.75 (s, 2H), 3.98 (s, 3H), 3.74 (s, 3H), 2.40 (s, 6H), 2.29 (s, 3H), 2.10 (s, 3H); LCMS: m/z 540.3 (M + H)⁺. |

Notes:
*Compounds 51-55 undergoes further deprotection step by using reagents such as TBAF (in 1M THF) and in the presence of suitable solvents at appropriate reaction conditions to give the respective compounds.
**Compounds 64 & 65 were prepared according to the protocol depicted in Example-IV with their appropriate starting compounds depicted below. Starting compounds for compound 64 & 65 are prepared according to the protocol described in intermediate-1.
Starting compound for 64 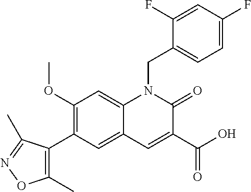 [SMILES: COc1cc2c(cc1-c1c(C)noc1C)cc(C(=O)O)c(=O)n2Cc1ccc(F)cc1F]
Starting compound for 65

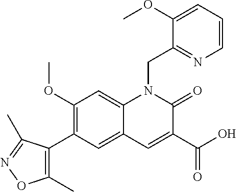 [SMILES: COc1cc2c(cc1-c1c(C)noc1C)cc(C(=O)O)c(=O)n2Cc1ncccc1OC]

### Example-V: 6-(3,5-Dimethylisoxazol-4-yl)-3-(1H-imidazol-2-yl)-7-methoxy-1-(pyridin-2-ylmethyl)quinolin-2(1H)-one (Compound-66)

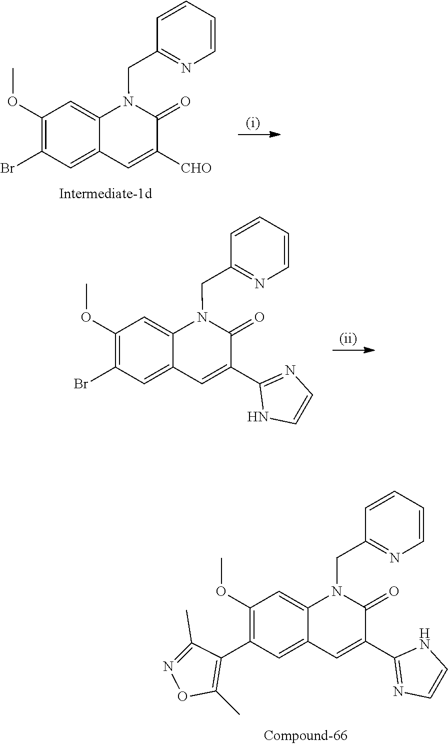 [SMILES: COc1cc2c(cc1Br)cc(C=O)c(=O)n2Cc1ccccn1 ; COc1cc2c(cc1Br)cc(-c1ncc[nH]1)c(=O)n2Cc1ccccn1 ; COc1cc2c(cc1-c1c(C)noc1C)cc(-c1ncc[nH]1)c(=O)n2Cc1ccccn1]

### Step-i: 6-Bromo-3-(1H-imidazol-2-yl)-7-methoxy-1-(pyridin-2-ylmethyl) quinolin-2(1H)-one

To a solution of 6-bromo-7-methoxy-2-oxo-1-(pyridin-2-ylmethyl)-1,2-dihydroquinoline-3-carbaldehyde (intermediate-1d) (0.3 g, 0.8 mmol) in EtOH (10 mL) were added glyoxal 40% (1.2 mL) and ammonium hydroxide (2.5 mL) followed by stirring at RT for 16 h. The mixture was diluted with EtOAc (100 ml), washed with water (100 mL) and brine (100 mL), dried over sodium sulphate and concentrated under reduced pressure. The residue was purified by column chromatography (60-120 silica gel and 2% MeOH in DCM as eluent) to afford the title compound as brown solid (0.2 g, 60%). ¹H NMR (400 MHz, DMSO-d₆) δ 12.27 (bs, 1H), 8.73 (s, 1H), 8.50 (d, J=4.4 Hz, 1H), 8.24 (s, 1H), 7.79-7.74 (m, 1H), 7.36 (d, J=8.3 Hz, 1H), 7.31-7.28 (m, 1H), 7.16 (s, 1H), 7.15 (s, 2H), 5.78 (s, 2H), 3.84 (s, 3H); LC-MS: m/z 411.0 (M+H)⁺.

### Step-ii: 6-(3,5-dimethylisoxazol-4-yl)-3-(1H-imidazol-2-yl)-7-methoxy-1-(pyridin-2-ylmethyl) quinolin-2(1H)-one (Compound-66)

The process of this step was adopted from step-(i) of Compound-2. ¹H NMR (400 MHz, DMSO-d₆): δ 12.26 (s, 1H), 8.79 (s, 1H), 8.53 (d, J=4.4 Hz, 1H), 7.86 (s, 1H), 7.81-7.76 (m, 1H), 7.41 (d, J=8.3 Hz, 1H), 7.33-7.76 (m, 1H), 7.19 (s, 1H), 7.17 (bs, 1H), 7.08 (s, 1H), 5.82 (s, 2H), 3.79 (s, 3H), 2.28 (s, 3H), 2.09 (s, 3H); LC-MS: m/z 428.2 (M+H)⁺.

### Example-VI: 6-(3,5-dimethylisoxazol-4-yl)-7-methoxy-3-(1-phenyl-1H-imidazol-2-yl)-1-(pyridin-2-ylmethyl)quinolin-2(1H)-one (Compound-67)

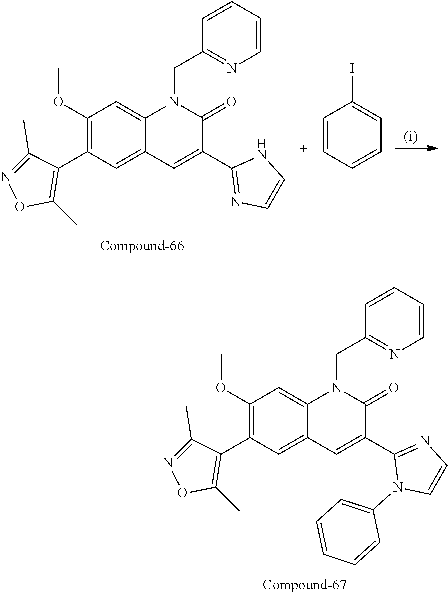 [SMILES: COc1cc2c(cc1-c1c(C)noc1C)cc(-c1ncc[nH]1)c(=O)n2Cc1ccccn1 ; Ic1ccccc1 ; COc1cc2c(cc1-c1c(C)noc1C)cc(-c1nccn1-c1ccccc1)c(=O)n2Cc1ccccn1]

The process of this step was adopted from step (i) of compound-15. ¹H NMR (400 MHz, DMSO-d₆) δ 8.51 (d, J=4.4 Hz, 1H), 8.31 (s, 1H), 7.75 (s, 1H), 7.68 (t, J=7.9 Hz, 1H), 7.63-7.61 (m, 1H), 7.43-7.23 (m, 7H), 7.16 (s, 1H), 6.73 (d, J=7.9 Hz, 1H), 5.46 (s, 2H), 3.78 (s, 3H), 2.26 (s, 3H), 2.08 (s, 3H); LC-MS: m/z 504.3 (M+H)⁺.

### Example-VII: 6-(3,5-Dimethylisoxazol-4-yl)-3-(hydroxydiphenylmethyl)-7-methoxy-1-(pyridin-2-ylmethyl)quinolin-2(1H)-one (Compound-68)

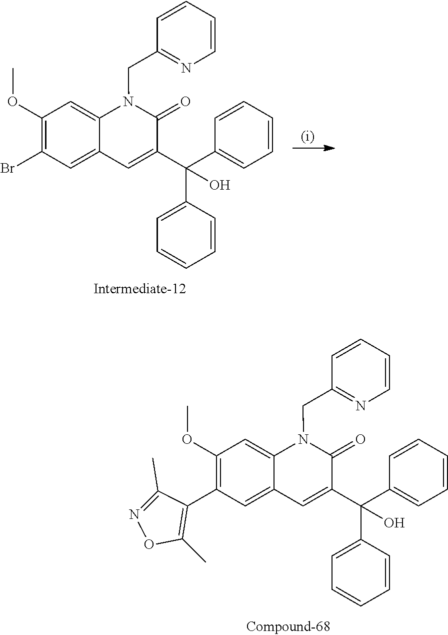 [SMILES: COc1cc2c(cc1Br)cc(C(O)(c1ccccc1)c1ccccc1)c(=O)n2Cc1ccccn1 ; COc1cc2c(cc1-c1c(C)noc1C)cc(C(O)(c1ccccc1)c1ccccc1)c(=O)n2Cc1ccccn1]

To a solution of 6-bromo-3-(hydroxydiphenylmethyl)-7-methoxy-1-(pyridin-2-ylmethyl)quinolin-2(1H)-one (intermediate-12) (0.08 g, 0.15 mmol) in 1,2-DME (4.0 mL) and H₂O (1.0 mL) were added 3,5-dimethylisoxazoleboronic acid (0.04 g, 0.30 mmol), sodium carbonate (0.05 g, 0.45 mmol) followed by degassing with nitrogen purging for 20 min. Then tetrakis triphenylphosphinepalladium (0.009 g, 0.015 mmol) was added followed by heating at 90° C. for 16 h. The mixture was diluted with EtOAc (50 ml), washed with water (50 mL) and brine (50 mL), dried over sodium sulphate and concentrated under reduced pressure. The residue obtained was purified by silica gel (60-120 mesh) column chromatography (elution 2% MeOH-DCM) to afford the title compound as white solid (0.02 g, 24%). ¹H NMR (400 MHz, DMSO-d₆) δ 8.52 (d, J=4.3 Hz, 1H), 7.77-7.75 (m, 1H), 7.57 (s, 1H), 7.36-7.24 (m, 13H), 7.18 (s, 1H), 6.80 (s, 1H), 5.68 (s, 2H), 3.75 (s, 3H), 2.20 (s, 3H), 2.01 (s, 3H).

### Example-VIII: 6-(3,5-Dimethylisoxazol-4-yl)-7-methoxy-1-(pyridin-2-ylmethyl)-3-(2,2,2-trifluoro-1-(4-fluorophenoxy)ethyl)quinolin-2(i 1H)-one (Compound-69)

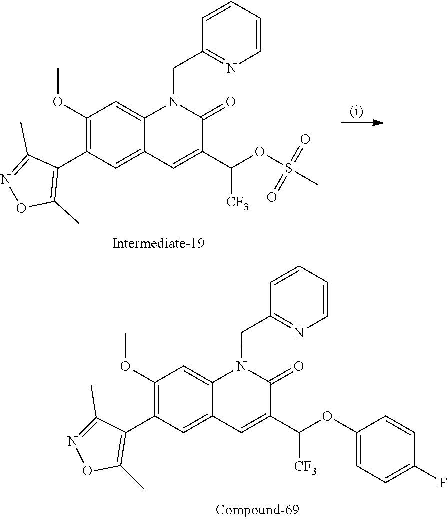 [SMILES: COc1cc2c(cc1-c1c(C)noc1C)cc(C(OS(C)(=O)=O)C(F)(F)F)c(=O)n2Cc1ccccn1 ; COc1cc2c(cc1-c1c(C)noc1C)cc(C(Oc1ccc(F)cc1)C(F)(F)F)c(=O)n2Cc1ccccn1]

To a solution of 1-(6-(3,5-dimethylisoxazol-4-yl)-7-methoxy-2-oxo-1-(pyridin-2-ylmethyl)-1,2-dihydroquinolin-3-yl)-2,2,2-trifluoroethylmethanesulfonate (intermediate-19) (0.15 g, 0.27 mmol) in DMF (3 mL) were added potassium carbonate (0.12 g, 0.83 mmol) and 4-fluorophenol (0.05 g, 0.41 mmol) followed by stirring at RT for 16 h. The mixture was diluted with EtOAc (50 mL), washed with water (50 mL) and brine (50 mL), dried over sodium sulphate and concentrated under reduced pressure. The residue was purified by preparative TLC to afford the title compound as off white solid (0.012 g, 8%). ¹H NMR (400 MHz, DMSO-d₆): δ 8.51 (d, J=3.9 Hz, 1H), 8.29 (s, 1H), 7.81 (s, 1H), 7.82-7.80 (m, 1H), 7.34-7.31 (m, 2H), 7.18-7.13 (m, 3H), 7.04-7.0 (m, 2H), 6.23-6.21 (m, 1H), 5.75 (s, 2H), 3.76 (s, 3H), 2.22 (s, 3H), 2.03 (s, 3H); LC-MS: m/z 554.2 (M+H)⁺.

The below compounds were prepared by a procedure similar to the one described in Example-VIII by using intermediate-19 as starting compound in the presence of suitable reagents and solvents at appropriate reaction conditions. The physiochemical characteristics of the compounds are also summarized.

| | | | Characterization Data |
|---|---|---|---|
| | | | ¹H NMR (400 MHz, DMSO-d₆)/LC- |
| No | Reactant | Obtained Compound | MS |
| 70 | 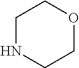 | 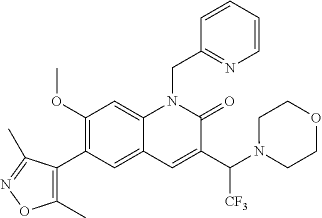 [SMILES: COc1cc2c(cc1-c1c(C)noc1C)cc(C(N1CCOCC1)C(F)(F)F)c(=O)n2Cc1ccccn1] | δ 8.52 (d, J = 4.9 Hz, 1H), 8.09 (s, 1H), 7.82-7.77 (m, 2H), 7.34-7.29 (m, 2H). 7.16 (s, 1H), 5.72 (s, 2H), 4.92 (q, J = 9.8 Hz, 1H), 3.77 (s, 3H), 3.58 (t, J = 4.9 Hz, 4H), 2.74-2.67 (m, 2H), 2.59- 2.53 (m, 2H), 2.26 (s, 3H), 2.07 (s, 3H); LC-MS: m/z 529.2 (M + H)⁺. |
| 71 | 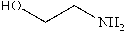 | 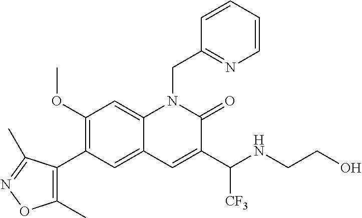 [SMILES: COc1cc2c(cc1-c1c(C)noc1C)cc(C(NCCO)C(F)(F)F)c(=O)n2Cc1ccccn1] | δ 8.53 (d, J = 4.4 Hz, 1H), 8.16 (s, 1H), 7.81-7.76 (m, 1H), 7.66 (s, 1H), 7.32- 7.29 (m, 2H), 7.16 (s, 1H), 5.70 (s, 2H). 4.83-4.75 (m, 1H), 4.54 (bs, 1H), 3.76 (s, 3H), 3.46-3.44 (m, 2H), 2.71- 2.76 (m, 1H), 2.67-2.63 (m, 2H), 2.25 (s, 3H), 2.06 (s, 3H); LC-MS: m/z 503.2 (M + H)⁺. |
| 72 | 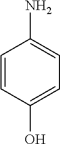 | 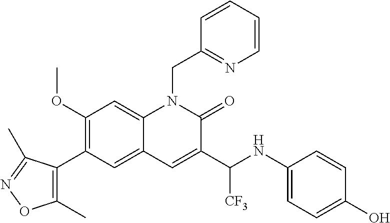 [SMILES: COc1cc2c(cc1-c1c(C)noc1C)cc(C(Nc1ccc(O)cc1)C(F)(F)F)c(=O)n2Cc1ccccn1] | δ 8.61 (s, 1H), 8.51 (d, J = 4.4 Hz, 1H), 8.23 (s, 1H), 7.81-7.76 (m, 1H), 7.57 (s, 1H), 7.32-7.28 (m, 2H), 7.17 (s, 1H), 6.61-6.53 (m, 4H), 6.12 (d, J = 10.3 Hz, 1H), 5.72 (s, 2H), 5.56-5.48 (m, 1H), 3.75 (s, 3H), 2.23 (s, 3H), 2.03 (s, 3H); LC-MS: m/z 551.2 (M + H)⁺. |

### Example-IX: 6-(3,5-Dimethylisoxazol-4-yl)-3-(1-ethoxy-2,2,2-trifluoroethyl)-7-methoxy-1-(pyridin-2-ylmethyl)quinolin-2(1H)-one (Compound-73)

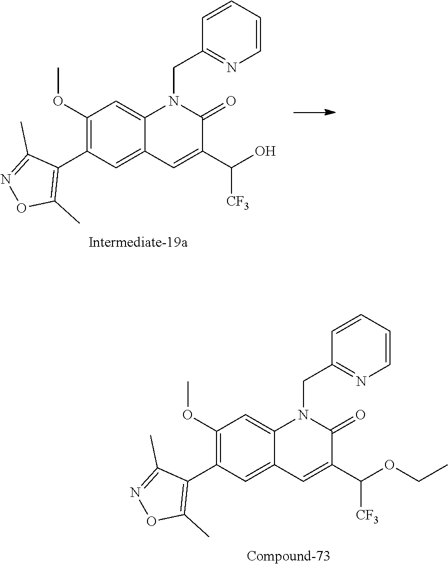 [SMILES: COc1cc2c(cc1-c1c(C)noc1C)cc(C(O)C(F)(F)F)c(=O)n2Cc1ccccn1 ; CCOC(c1cc2cc(-c3c(C)noc3C)c(OC)cc2n(Cc2ccccn2)c1=O)C(F)(F)F]

To a cooled solution of 6-(3,5-dimethylisoxazol-4-yl)-7-methoxy-1-(pyridin-2-ylmethyl)-3-(2,2,2-trifluoro-1-hydroxyethyl)quinolin-2(1H)-one (intermediate-19a) (0.15 g, 0.32 mmol) in DMF (5 mL) was added sodium hydride 60% (0.06 g, 0.98 mmol) followed by stirring at RT for 30 min. Then iodoethane (0.04 mL, 0.65 mmol) was added followed by stirring at RT for 16 h. The mixture was quenched with ice water and extracted with EtOAc (50 mL×2). Combined organic phase was washed with brine (50 mL), dried over sodium sulphate and concentrated under reduced pressure. The residue was purified by silica gel (60-120 mesh) column chromatography (elution 20% EtOAc-hexane) to afford the title compound as pale brown solid (0.015 g, 10%). ¹H NMR (400 MHz, DMSO-d₆): δ 8.52 (d, J=4.4 Hz, 1H), 8.15 (s, 1H), 7.83 (s, 1H), 7.80-7.76 (m, 1H), 7.32-7.29 (m, 2H), 7.15 (s, 1H), 5.70 (s, 2H), 5.37-5.35 (m, 1H), 3.76 (s, 3H), 3.67-3.63 (m, 2H), 2.25 (s, 3H), 2.06 (s, 3H), 1.20-1.17 (m, 3H); LC-MS: m/z 488.2 (M+H)⁺.

The below compound was prepared by a procedure similar to the one described in Example-IX by using intermediate-19a in presence of appropriate reactants and solvents at appropriate reaction conditions. The physiochemical characteristic of the compound is also summarized.

| No | Reactant | Obtained Compound | Characterization Data |
|---|---|---|---|
| 74 | 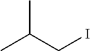 | 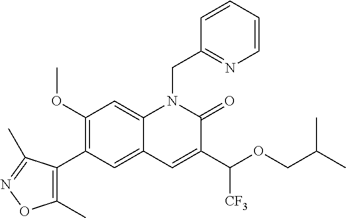 [SMILES: COc1cc2c(cc1-c1c(C)noc1C)cc(C(OCC(C)C)C(F)(F)F)c(=O)n2Cc1ccccn1] | ¹H NMR (400 MHz, DMSO-d₆): δ 8.53 (d, J = 4.8 Hz, 1H), 8.14 (s, 1H), 7.84 (s, 1H), 7.81-7.77 (m, 1H), 7.33-7.29 (m, 2H), 7.16 (s, 1H), 5.70 (s, 2H), 5.34 (q, J = 6.8 Hz, 1H), 3.76 (s, 3H), 3.38 (dd, J1 = 1.5 Hz, J2 = 6.4 Hz, 2H), 2.26 (s, 3H), 2.06 (s, 3H), 1.89-1.82 (m, 1H), 0.89 (d, J = 6.4 Hz, 6H); LC-MS: m/z 516.0 (M + H) |

### Example-X: 6-(3,5-dimethylisoxazol-4-yl)-3-(3-ethyl-1,2,4-oxadiazol-5-yl)-7-methoxy-1-(pyridin-2-ylmethyl)quinolin-2(1H)-one (Compound-75)

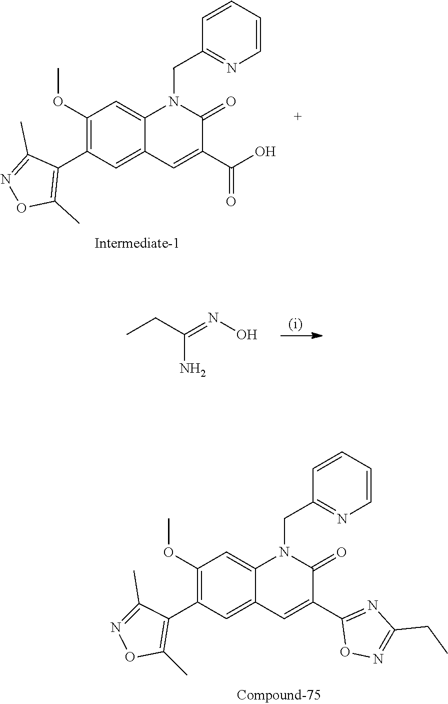

To a solution of 6-(3,5-dimethylisoxazol-4-yl)-7-methoxy-2-oxo-1-(pyridin-2-ylmethyl)-1,2-dihydroquinoline-3-carboxylic acid (intermediate-1) (0.05 g, 0.12 mmol) in DMF (3 mL) were added HATU (0.05 g, 0.135 mmol) and diisopropyl ethyl amine (0.025 mL, 0.18 mmol) followed by stirring at RT for 10 min. Then (Z)—N′-hydroxy-propionimidamide was added followed by stirring at RT for 16 h. The mixture was diluted with EtOAc (50 mL), washed with water (50 mL) and brine (50 mL), dried over sodium sulphate and concentrated under reduced pressure. The residue was purified by preparative TLC to afford the title compound as off white solid (0.015 g, 13%). ¹H NMR (400 MHz, DMSO-d₆) δ 8.93 (s, 1H), 8.52 (d, J=4.4 Hz, 1H), 7.95 (s, 1H), 7.82-7.78 (m, 1H), 7.44 (d, J=8.3 Hz, 1H), 7.33-7.30 (m, 1H), 7.19 (s, 1H), 5.80 (s, 2H), 3.82 (s, 3H), 2.80 (q, J=7.8 Hz, 2H), 2.26 (s, 3H), 2.09 (s, 3H), 1.30 (t, J=7.8 Hz, 3H); LC-MS: m/z 458.2 (M+H)+.

### Example-XI: 3-Benzoyl-6-(3,5-dimethylisoxazol-4-yl)-7-methoxy-1-(pyridin-2-yl-methyl)quinolin-2(1H)-one (Compound-76)

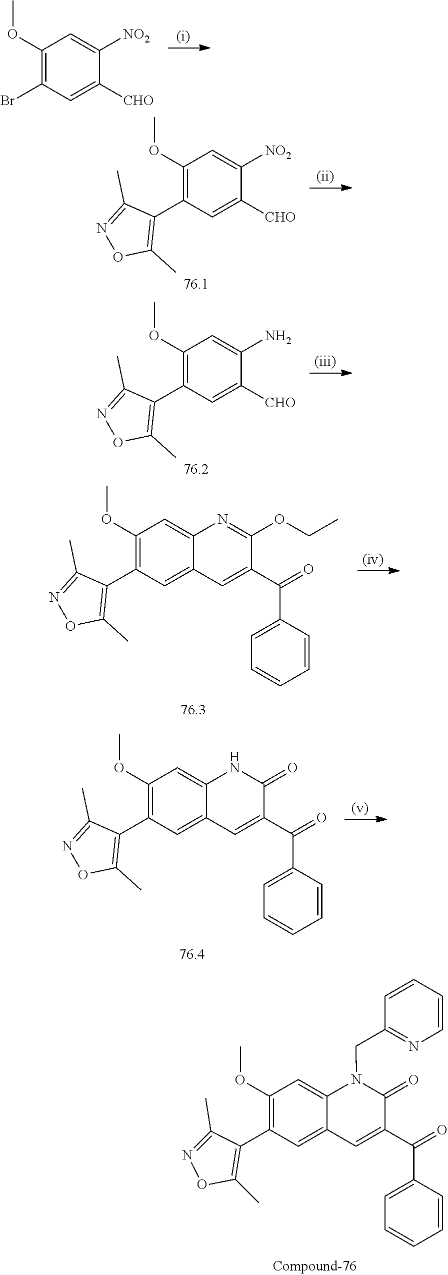

### Step-i: 5-(3,5-dimethylisoxazol-4-yl)-4-methoxy-2-nitrobenzaldehyde (76.1)

The process of this step was adopted from step-(i) of compound-2. ¹H NMR (400 MHz, DMSO-d₆): δ 10.09 (s, 1H), 7.86 (s, 1H), 7.83 (s, 1H), 3.99 (s, 3H), 2.31 (s, 3H), 2.11 (s, 3H); LC-MS: m/z 277.1 (M+H)⁺.

### Step-ii: 2-amino-5-(3,5-dimethylisoxazol-4-yl)-4-methoxybenzaldehyde (76.2)

To a solution of 5-(3,5-dimethylisoxazol-4-yl)-4-methoxy-2-nitrobenzaldehyde (2.0 g, 7.24 mmol) in EtOH (20 mL) was added sodium dithionate (7.46 g, 36.2 mmol) followed by stirring at 80° C. for 3 h. The mixture was filtered and washed with EtOAc and concentrated under reduced pressure. The residue was used in the further step without purification (1.2 g). LC-MS: m/z 247.1 (M+H)⁺.

### Step-iii: (6-(3,5-dimethylisoxazol-4-yl)-2-ethoxy-7-methoxyquinolin-3-yl)-(phenyl)methanone (76.3)

To a solution of 2-amino-5-(3,5-dimethylisoxazol-4-yl)-4-methoxybenzaldehyde (1.0 g, 4.06 mmol) in EtOH (10 mL) was added ethyl 3-oxo-3-phenylpropanoate (1.56 mL, 8.13 mmol) and piperidine (0.04 mL, 0.41 mmol) followed by refluxing for 16 h. The mixture was concentrated under reduced pressure. The residue was used further without purification (0.7 g, 43%); LC-MS: m/z 402.8 (M+H)⁺.

### Step-iv: 3-benzoyl-6-(3,5-dimethylisoxazol-4-yl)-7-methoxyquinolin-2(1H)-one (76.4)

To a solution of (6-(3,5-dimethylisoxazol-4-yl)-2-ethoxy-7-methoxyquinolin-3-yl)(phenyl)methanone (0.7 g, 1.74 mmol) in 1,4-dioxane (10 mL) was added 3 N HCl (3 mL) followed by refluxing for 16 h. The mixture was poured into saturated NaHCO₃, extracted with EtOAc (100 mL), washed with water (100 mL) and brine (100 mL), dried over sodium sulphate and concentrated under reduced pressure. The residue was used further without purification (0.4 g).

### Step-v: 3-benzoyl-6-(3,5-dimethylisoxazol-4-yl)-7-methoxy-1-(pyridin-2-ylmethyl)quinolin-2(1H)-one (76.5)

The process of this was adopted from step-e of intermediate-2. ¹H NMR (400 MHz, DMSO-d₆) δ 8.89 (s, 1H), 8.54 (d, J=4.9 Hz, 1H), 8.12 (s, 1H), 7.50-7.49 (m, 1H), 7.63-7.61 (m, 3H), 7.45-7.44 (m, 3H), 7.40-7.35 (m, 1H), 7.09 (d, J=7.9 Hz, 1H), 5.28 (s, 2H), 3.99 (s, 3H), 2.35 (s, 3H), 2.15 (s, 3H); LC-MS: m/z 466.2 (M+H)⁺.

### Example-XII: 2-(6-(3,5-Dimethylisoxazol-4-yl)-7-methoxy-2-oxo-1-(pyridin-2-yl-methyl)-1, 2-dihydroquinolin-3-yl) acetic Acid (Compound-77)

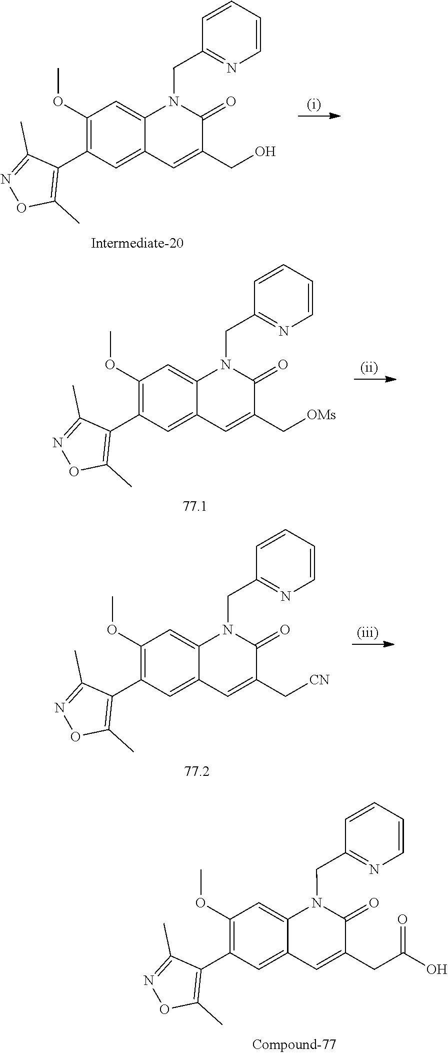 [SMILES: COc1cc2c(cc1-c1c(C)noc1C)cc(CO)c(=O)n2Cc1ccccn1 ; COc1cc2c(cc1-c1c(C)noc1C)cc(COS(C)(=O)=O)c(=O)n2Cc1ccccn1 ; COc1cc2c(cc1-c1c(C)noc1C)cc(CC#N)c(=O)n2Cc1ccccn1 ; COc1cc2c(cc1-c1c(C)noc1C)cc(CC(=O)O)c(=O)n2Cc1ccccn1]

### Step-i: (6-(3,5-Dimethylisoxazol-4-yl)-7-methoxy-2-oxo-1-(pyridin-2-ylmethyl)-1,2-dihydro quinolin-3-yl)methyl Methanesulfonate (77.1)

The process of this step was adopted from step-a of intermediate-5.

### Step-ii: 2-(6-(3,5-Dimethylisoxazol-4-yl)-7-methoxy-2-oxo-1-(pyridin-2-yl-methyl)-1,2-dihydro quinolin-3-yl)acetonitrile (77.2)

To a cooled solution of (6-(3,5-dimethylisoxazol-4-yl)-7-methoxy-2-oxo-1-(pyridin-2-ylmethyl)-1,2-dihydroquinolin-3-yl)methylmethanesulfonate (1.08 g, 2.30 mmol) in DMF (10 mL) was added potassium cyanide (0.3 g, 4.60 mmol) followed by stirring at RT for 16 h. The mixture was poured into ice water and extracted with EtOAc (100×2), dried over sodium sulphate and concentrated under reduced pressure. The residue was purified by silica gel (60-120 mesh) column chromatography (elution 30-40% EtOAc-hexane) to afford the title compound as off white solid, (0.6 g). ¹H NMR (400 MHz, DMSO-d₆) δ 8.51 (d, J=3.9 Hz, 1H), 8.06 (s, 1H), 7.89-7.76 (m, 1H), 7.72 (s, 1H), 7.36 (d, J=7.8 Hz, 1H), 7.32-7.29 (m, 1H), 7.14 (s, 1H), 5.71 (s, 2H), 3.89 (s, 2H), 3.75 (s, 3H), 2.26 (s, 3H), 2.07 (s, 3H); LC-MS: m/z 401.1 (M+H)+.

### Step-iii: 2-(6-(3,5-Dimethylisoxazol-4-yl)-7-methoxy-2-oxo-1-(pyridin-2-yl-methyl)-1,2-dihydro quinolin-3-yl)acetic Acid (Compound-77)

A solution of 2-(6-(3,5-dimethylisoxazol-4-yl)-7-methoxy-2-oxo-1-(pyridin-2-ylmethyl)-1,2-dihydroquinolin-3-yl)acetonitrile (0.35 g) in 6 N HCl (5 mL) was heated at 100° C. for 6 h. The mixture was poured into saturated NaHCO₃ (pH-8), acidified with citric acid solution, extracted with EtOAc (100×2), dried over sodium sulphate and concentrated in vacuo. The residue was purified by silica gel (60-120 mesh) column chromatography (elution 2-4% MeOH-DCM) to afford the title compound as off white solid (0.1 g, 27%). ¹H NMR (400 MHz, DMSO-d₆) δ 12.29 (bs, 1H), 8.53 (d, J=4.4 Hz, 1H), 7.86 (s, 1H), 7.80-7.75 (m, 1H), 7.60 (s, 1H), 7.32-7.27 (m, 2H), 7.13 (s, 1H), 5.67 (s, 2H), 3.74 (s, 3H), 3.53 (s, 2H), 2.25 (s, 3H), 2.09 (s, 3H); LC-MS: m/z 420.2 (M+H)+.

The below compound was prepared by a procedure similar to the one described in Example-XII by using intermediates 21 and 21 a as starting compound in the presence of suitable reagents and solvents at appropriate reaction conditions. The physiochemical characteristic of the compound is also summarized.

| | | | Characterization Data |
|---|---|---|---|
| | | | ¹H NMR (400 MHz, DMSO-d₆)/ |
| No | Intermediate | Obtained Compound | LC-MS: |
| 78 | 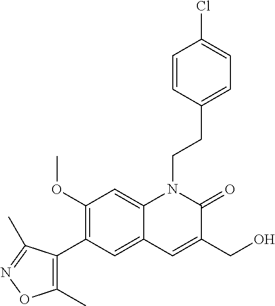 | 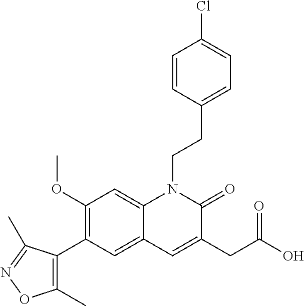 [SMILES: COc1cc2c(cc1-c1c(C)noc1C)cc(CC(=O)O)c(=O)n2CCc1ccc(Cl)cc1] | ¹H NMR (400 MHz, DMSO-d₆) δ 12.2 (bs, 1H), 7.79 (s, 1H), 7.56 (s, 1H), 7.36-7.31 (m, 4H), 6.97 (s, 1H), 4.52 (t, J = 7.3 Hz, 2H), 3.92 (s, 3H), 3.48 (s, 2H), 2.97 (t, J = 7.3 Hz, 2H), 2.27 (s, 3H), 2.08 (s, 3H); LC-MS: m/z 467.0 (M + H)⁺. |
| 79 | 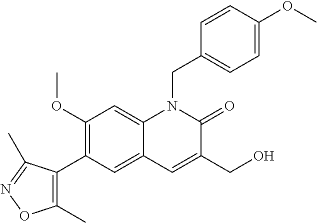 | 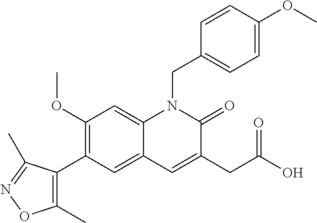 [SMILES: COc1ccc(Cn2c(=O)c(CC(=O)O)cc3cc(-c4c(C)noc4C)c(OC)cc32)cc1] | ¹H NMR (400 MHz, DMSO-d₆): δ 12.28 (s, 1H), 7.83 (s, 1H), 7.57 (s, 1H), 7.30 (d, J = 8.3 Hz, 2H), 7.03 (s, 1H), 6.89 (d, J = 8.8 Hz, 2H), 5.53 (s, 2H), 3.79 (s, 3H), 3.70 (s, 3H), 3.54 (s, 2H), 2.24 (s, 3H), 2.05 (s, 3H); LC-MS: m/z 449.2 (M + H)⁺. |

### Example-XIII: 2-(4-(6-(3,5-Dimethylisoxazol-4-yl)-7-methoxy-2-oxo-1-(pyridin-2-yl-methyl)-1,2-dihydroquinolin-3-yl)-1H-pyrazol-1-yl)-N-(2-hydroxyethyl)acetamide (Compound-80) and 2-(4-(6-(3,5-dimethylisoxazol-4-yl)-7-methoxy-2-oxo-1-(pyridin-2-ylmethyl)-1,2-dihydroquinolin-3-yl)-1H-pyrazol-1-yl)acetic acid (Compound-81)

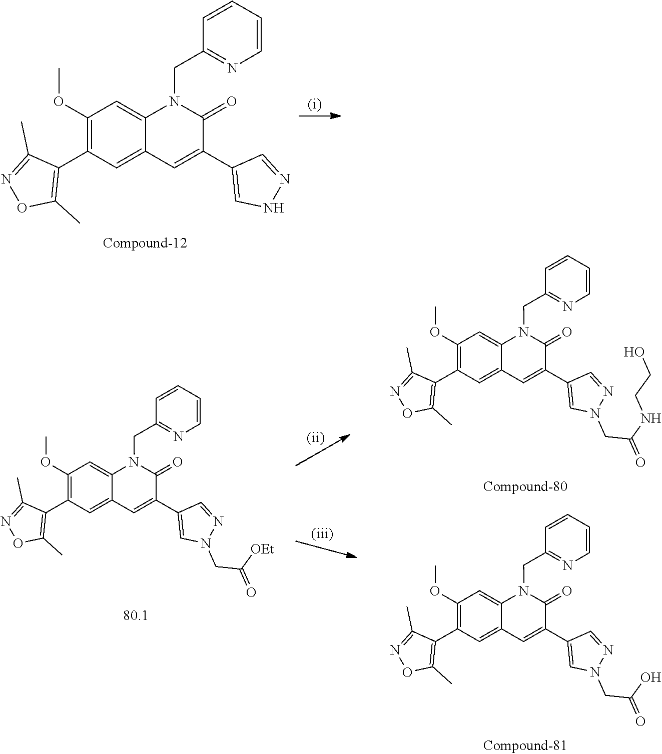 [SMILES: COc1cc2c(cc1-c1c(C)noc1C)cc(-c1cn[nH]c1)c(=O)n2Cc1ccccn1 ; CCOC(=O)Cn1cc(-c2cc3cc(-c4c(C)noc4C)c(OC)cc3n(Cc3ccccn3)c2=O)cn1 ; COc1cc2c(cc1-c1c(C)noc1C)cc(-c1cnn(CC(=O)NCCO)c1)c(=O)n2Cc1ccccn1 ; COc1cc2c(cc1-c1c(C)noc1C)cc(-c1cnn(CC(=O)O)c1)c(=O)n2Cc1ccccn1]

### Step-i: Ethyl 2-(4-(6-(3,5-dimethylisoxazol-4-yl)-7-methoxy-2-oxo-1-(pyridin-2-ylmethyl)-1,2-dihydroquinolin-3-yl)-1H-pyrazol-1-yl)acetate (80.1)

The process of this step was adopted from step-e of intermediate-2. ¹H NMR (400 MHz, DMSO-d₆) δ 8.53-8.51 (m, 2H), 8.34 (s, 1H), 8.11 (s, 1H), 7.85-7.75 (m, 1H), 7.60 (s, 1H), 7.36 (d, J=7.8 Hz, 1H), 7.35-7.25 (m, 1H), 7.15 (s, 1H), 5.76 (s, 2H), 5.13 (s, 2H), 4.74-4.16 (m, 2H), 3.76 (s, 3H), 2.27 (s, 3H), 2.08 (s, 3H), 1.25-1.22 (m, 3H); LC-MS: m/z 514.2 (M+H)⁺.

### Step-ii: 2-(4-(6-(3,5-Dimethylisoxazol-4-yl)-7-methoxy-2-oxo-1-(pyridin-2-ylmethyl)-1,2-dihydroquinolin-3-yl)-1H-pyrazol-1-yl)-N-(2-hydroxyethyl)acetamide (compound-80)

A solution of ethyl 2-(4-(6-(3, 5-dimethylisoxazol-4-yl)-7-methoxy-2-oxo-1-(pyridin-2-ylmethyl)-1,2-dihydroquinolin-3-yl)-1H-pyrazol-1-yl)acetate (0.06 g) in 2-aminoethane-1-ol (0.5 mL) in a sealed tube was heated at 90° C. for 4 h. The mixture was poured into crushed ice, the solids were filtered, washed thoroughly with water, and dried under reduced pressure to afford the title compound as off white solid (0.02 g, 32%). ¹H NMR (400 MHz, DMSO-d₆) δ 8.55-8.50 (m, 1H), 8.49 (s, 1H), 8.31 (s, 1H), 8.20-8.10 (m, 1H), 8.06 (s, 1H), 7.85-7.75 (m, 1H), 7.58 (s, 1H), 7.40-7.20 (m, 2H), 7.13 (s, 1H), 5.74 (s, 2H), 4.83 (s, 2H), 4.80-4.73 (m, 1H), 3.74 (s, 3H), 3.41-3.40 (m, 2H), 3.15-3.14 (m, 2H), 2.25 (s, 3H), 2.06 (s, 3H); LC-MS: m/z 529.4 (M+H)⁺.

### Step-iii: 2-(4-(6-(3,5-Dimethylisoxazol-4-yl)-7-methoxy-2-oxo-1-(pyridin-2-ylmethyl)-1,2-dihydroquinolin-3-yl)-1H-pyrazol-1-yl)acetic Acid (Compound-81)

To a solution of ethyl 2-(4-(6-(3,5-dimethylisoxazol-4-yl)-7-methoxy-2-oxo-1-(pyridin-2-ylmethyl)-1,2-dihydroquinolin-3-yl)-1H-pyrazol-1-yl)acetate (0.1 g, 0.19 mmol) in MeOH (4 mL) were added sodium hydroxide (0.02 g, 0.39 mmol) in water (1 mL) followed by stirring at RT for 1 h. The mixture was concentrated to remove methanol, diluted with water, acidified with 1N HCl and then extracted with EtOAc (50 ml). The organic layer was washed with brine (50 mL), dried over sodium sulphate and concentrated under reduced pressure. The obtained solid was washed with diethyl ether and EtOAc and filtered off to afford the title product as brown solid (0.08 g, 85%). ¹H NMR (400 MHz, DMSO-d₆): δ 13.08 (bs, 1H), 8.53 (d, J=4.4 Hz, 1H), 8.49 (s, 1H), 8.33 (s, 1H), 8.09 (s, 1H), 7.79-7.74 (m, 1H), 7.60 (s, 1H), 7.36 (d, J=7.8 Hz, 1H), 7.31-7.28 (m, 1H), 7.15 (s, 1H), 5.76 (d, J=1.9 Hz, 2H), 5.03 (s, 2H), 3.76 (s, 3H), 2.27 (s, 3H), 2.08 (s, 3H); LC-MS: m/z 486.2 (M+H)⁺.

### Example-XIV: 6-(3,5-Dimethylisoxazol-4-yl)-7-methoxy-2-oxo-N-(pyridin-2-yl)-1-(pyridin-2-ylmethyl)-1,2-dihydroquinoline-3-sulfonamide (Compound-82)

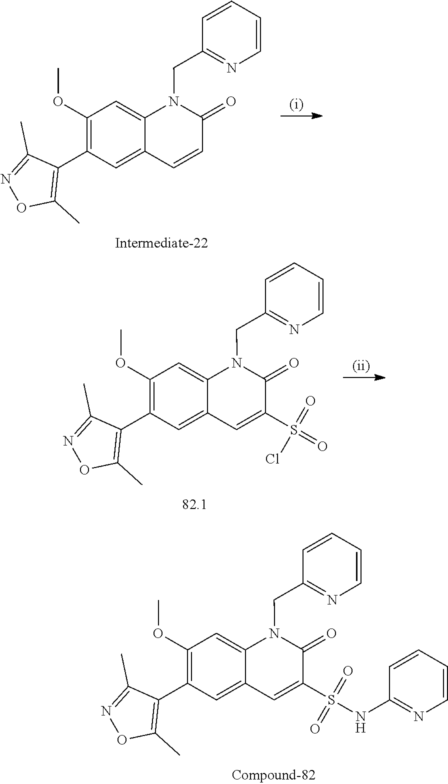 [SMILES: COc1cc2c(ccc(=O)n2Cc2ccccn2)cc1-c1c(C)noc1C ; COc1cc2c(cc1-c1c(C)noc1C)cc(S(=O)(=O)Cl)c(=O)n2Cc1ccccn1 ; COc1cc2c(cc1-c1c(C)noc1C)cc(S(=O)(=O)Nc1ccccn1)c(=O)n2Cc1ccccn1]

### Step-i: 6-(3,5-Dimethylisoxazol-4-yl)-7-methoxy-2-oxo-1-(pyridin-2-ylmethyl)-1,2-dihydro quinoline-3-sulfonyl Chloride (82.1)

A solution of 6-(3,5-dimethylisoxazol-4-yl)-7-methoxy-1-(pyridin-2-yl-methyl)quinolin-2(1H)-one (intermediate-22) (0.3 g) in chlorosulfonic acid (3 mL) was heated at 70° C. for 2 h. The mixture was poured into crushed ice, solids were filtered, washed the solid thoroughly with water and vacuum dried to afford the title compound as brown solid (0.1 g). ¹H NMR (400 MHz, DMSO-d₆) δ 8.78 (d, J=5.4 Hz, 1H), 8.39 (s, 1H), 8.20-8.16 (m, 1H), 7.80 (s, 1H), 7.72-7.68 (m, 1H), 7.51 (d, J=7.9 Hz, 1H), 7.10 (s, 1H), 5.78 (s, 2H), 3.80 (s, 3H), 2.27 (s, 3H), 2.09 (s, 3H); LC-MS: m/z 459.8 (M+H)⁺.

### Step-ii: 6-(3,5-Dimethylisoxazol-4-yl)-7-methoxy-2-oxo-N-(pyridin-2-yl)-1-(pyridin-2-ylmethyl)-1,2-dihydroquinoline-3-sulfonamide (Compound-82)

To a cold solution of 6-(3,5-dimethylisoxazol-4-yl)-7-methoxy-2-oxo-1-(pyridin-2-ylmethyl)-1,2-dihydroquinoline-3-sulfonyl chloride (0.1 g, 0.22 mmol) in DCM (2 mL) were added triethyl amine (0.09 mL, 0.65 mmol) and 2-amino pyridine (0.03 g, 0.33 mmol) followed by stirring at RT for 3 h. The mixture was diluted with DCM (50 ml), washed with water (50 mL) and brine (50 mL), dried over sodium sulphate and concentrated under reduced pressure. The residue was purified on preparative TLC plate to afford the title product as brown solid (0.006 g, 5%). ¹H NMR (400 MHz, DMSO-d₆): δ 8.76 (s, 1H) 8.50-8.48 (m, 1H), 8.06-8.04 (m, 1H), 7.96-7.90 (m, 2H), 7.77-7.69 (m, 2H), 7.33-7.30 (m, 1H), 7.23-7.21 (m, 1H), 7.11 (s, 1H), 7.69 (d, J=7.4 Hz, 1H), 6.87-6.85 (m, 1H), 5.65 (s, 2H), 3.83 (s, 3H), 2.27 (s, 3H), 2.09 (s, 3H); LC-MS: m/z 518.5 (M+H)⁺.

### Example-XV: 6-(3,5-Dimethylisoxazol-4-yl)-3-(6-hydroxy-1H-benzo[d] imidazol-2-yl)-7-methoxy-1-(pyridin-2-ylmethyl)quinolin-2(1H)-one (Compound-83)

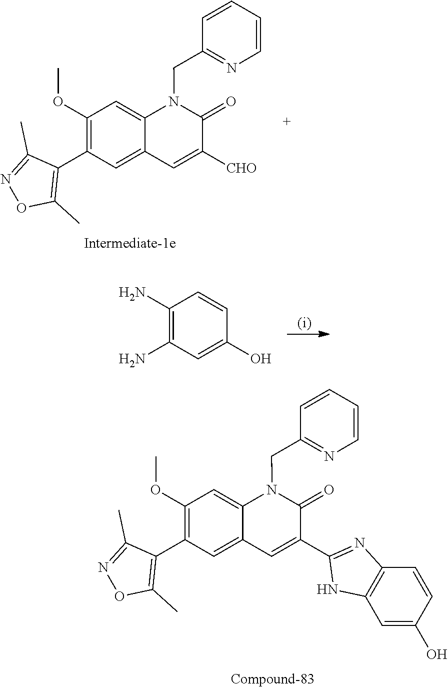 [SMILES: COc1cc2c(cc1-c1c(C)noc1C)cc(C=O)c(=O)n2Cc1ccccn1 ; Nc1ccc(O)cc1N ; COc1cc2c(cc1-c1c(C)noc1C)cc(-c1nc3ccc(O)cc3[nH]1)c(=O)n2Cc1ccccn1]

To a stirred solution of 6-(3,5-dimethylisoxazol-4-yl)-7-methoxy-2-oxo-1-(pyridin-2-ylmethyl)-1,2-dihydroquinoline-3-carbaldehyde (intermediate-1e) (0.1 g, 0.25 mmol) in acetic acid (5 mL) was added 3,4-diaminophenol (0.04 g, 0.3 mmol) followed by heating to reflux for 16 h. The mixture was concentrated to remove acetic acid. The residue was diluted with EtOAc and washed with water (50 mL), saturated NaHCO₃ (50 ml) and brine (50 mL), dried over sodium sulphate and concentrated under reduced pressure. The residue was purified by preparative TLC to afford the title compound as a brown solid (0.01 g, 8%). ¹H NMR (400 MHz, DMSO-d₆) δ 12.14-12.33 (m, 1H), 9.17-9.0 (m, 2H), 8.53 (d, J=4.9 Hz, 1H), 7.94 (s, 1H), 7.81 (t, J=7.4 Hz, 1H), 7.47-7.40 (m, 2H), 7.33-7.30 (m, 1H), 7.23 (s, 1H), 7.04-6.95 (m, 1H), 6.69 (d, J=8.3 Hz, 1H), 5.85 (s, 2H), 3.82 (s, 3H), 2.30 (s, 3H), 2.11 (s, 3H); LC-MS: m/z 494.2 (M+H)⁺.

### Example-XVI: 3-(Azetidine-1-carbonyl)-1-(4-chlorobenzyl)-6-(3,5-dimethyl isoxazol-4-yl)-7-methoxyquinolin-2(1H)-one (Compound-84)

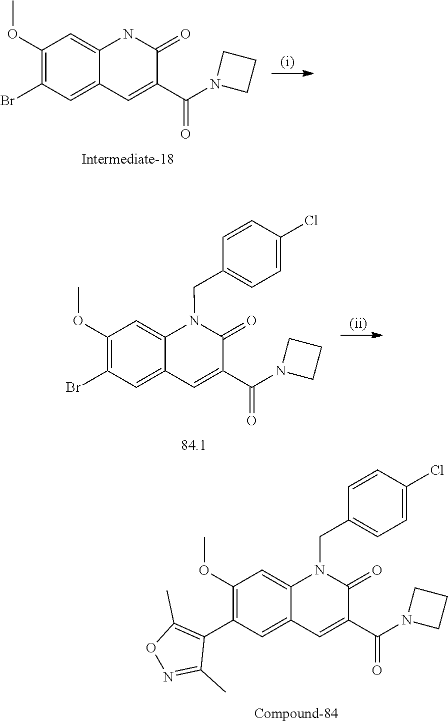 [SMILES: COc1cc2[nH]c(=O)c(C(=O)N3CCC3)cc2cc1Br ; COc1cc2c(cc1Br)cc(C(=O)N1CCC1)c(=O)n2Cc1ccc(Cl)cc1 ; COc1cc2c(cc1-c1c(C)noc1C)cc(C(=O)N1CCC1)c(=O)n2Cc1ccc(Cl)cc1]

### Step-i: 3-(Azetidine-1-carbonyl)-6-bromo-1-(4-chlorobenzyl)-7-methoxyquinolin-2(1H)-one (84.1)

The process of this step was adopted from step-e of intermediate-2. ¹H NMR (400 MHz, DMSO-d₆): δ 8.12 (d, J=2.9 Hz, 2H), 7.39 (d, J=8.3 Hz, 2H), 7.30 (d, J=9.7 Hz, 2H), 6.95 (s, 1H), 5.75 (s, 2H), 4.07-3.99 (m, 4H), 3.85 (s, 3H), 2.23 (t, J=7.8 Hz, 2H); LC-MS: m/z 463.0 (M+2H)²⁺.

### Step-ii: 3-(Azetidine-1-carbonyl)-1-(4-chlorobenzyl)-6-(3,5-dimethylisoxazol-4-yl)-7-methoxy quinolin-2(1H)-one (Compound 84)

The process of this step was adopted from step-a of intermediate-2. ¹H NMR (400 MHz, DMSO-d₆): δ 8.13 (s, 1H), 7.72 (s, 1H), 7.41-7.35 (m, 4H), 6.96 (s, 1H), 5.60 (s, 2H), 4.06 (t, J=7.4 Hz, 2H), 4.0 (t, J=7.4 Hz, 2H), 3.76 (s, 3H), 2.26-2.18 (m, 2H), 2.26 (s, 3H), 2.04 (s, 3H); LC-MS: m/z 478.2 (M+H)⁺.

The below compounds were prepared by a procedure similar to the one described in Example-XVI by using intermediate-18 as starting compound and reacting with appropriate reactants B in the presence of suitable reagents, catalysts and solvents at appropriate reaction conditions. The physiochemical characteristics of the compounds are also summarized.

| No | Reactant B | Obtained Compound | Characterization Data |
|---|---|---|---|
| 85 | 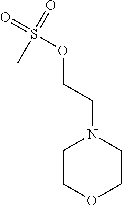 | 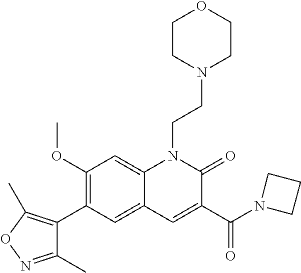 [SMILES: COc1cc2c(cc1-c1c(C)noc1C)cc(C(=O)N1CCC1)c(=O)n2CCN1CCOCC1] | ¹H NMR (400 MHz, CDCl₃): δ 8.02 (s, 1H), 7.35 (s, 1H), 6.91 (s, 1H), 4.49 (t, J = 7.3 Hz, 2H), 4.25-4.18 (m, 4H), 3.94 (s, 3H), 3.75-3.73 (m, 4H), 2.72 (t, J = 7.3 Hz, 2H), 2.65-2.62 (m, 4H), 2.36- 2.30 (m, 2H), 2.30 (s, 3H), 2.16 (s, 3H); LC-MS: m/z 467.3 (M + H)⁺. |
| 86 | 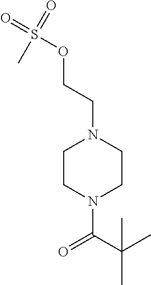 | 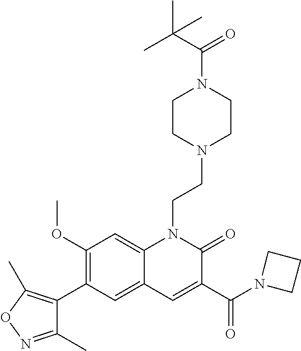 [SMILES: COc1cc2c(cc1-c1c(C)noc1C)cc(C(=O)N1CCC1)c(=O)n2CCN1CCN(C(=O)C(C)(C)C)CC1] | ¹H NMR (400 MHz, CDCl₃): δ 8.01 (s, 1H), 7.35 (s, 1H), 6.92 (s, 1H), 4.54 (bs, 2H), 4.25-4.17 (m, 4H), 3.95 (s, 3H), 3.71 (bs, 4H), 2.79-2.64 (m, 6H), 2.35-2.33 (m, 2H), 2.30 (s, 3H), 2.16 (s, 3H), 1.28 (s, 9H); LC-MS: m/z 550.3 (M + H)⁺. |
| 87 | 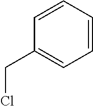 | 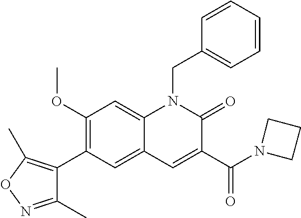 [SMILES: COc1cc2c(cc1-c1c(C)noc1C)cc(C(=O)N1CCC1)c(=O)n2Cc1ccccc1] | ¹H NMR (400 MHz, DMSO-d₆): δ 8.15 (s, 1H), 7.73 (s, 1H), 7.37-7.34 (m, 4H), 7.29-7.26 (m, 1H), 7.00 (s, 1H). 5.63 (s, 2H), 4.10 (t, J = 7.4 Hz, 2H), 4.02 (t, J = 7.3 Hz, 2H), 3.78 (s, 3H), 2.28-2.24 (m, 2H), 2.27 (s, 3H), 2.05 (s, 3H); LC- MS: m/z 444.2 (M + H)⁺. |
| 88 | 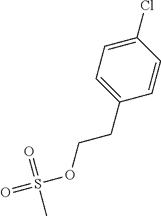 | 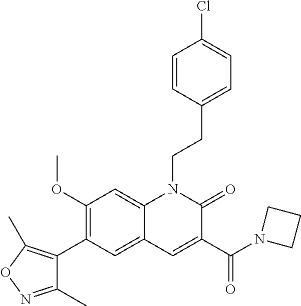 [SMILES: COc1cc2c(cc1-c1c(C)noc1C)cc(C(=O)N1CCC1)c(=O)n2CCc1ccc(Cl)cc1] | ¹H NMR (400 MHz, CDCl₃): δ 8.02 (s, 1H), 7.35 (s, 1H), 7.28-7.26 (m, 2H), 7.19 (d, J = 8.3 Hz, 2H), 6.70 (s, 1H), 4.52 (t, J = 7.8 Hz, 2H), 4.22 (t, J = 7.8 Hz, 2H), 4.11 (t, J = 7.3 Hz, 2H), 3.88 (s, 3H), 3.06 (t, J = 7.3 Hz, 2H), 2.34-2.32 (m, 2H), 2.30 (s, 3H), 2.15 (s, 3H); LC-MS: m/z 492.1 (M + H)⁺. |
| 89 | 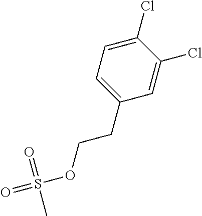 | 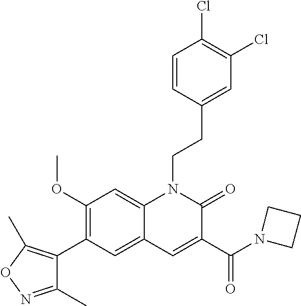 [SMILES: COc1cc2c(cc1-c1c(C)noc1C)cc(C(=O)N1CCC1)c(=O)n2CCc1ccc(Cl)c(Cl)c1] | ¹H NMR (400 MHz, DMSO-d₆): δ 8.06 (s, 1H), 7.70 (s, 1H), 7.53 (s, 1H), 7.50 (d, J = 8.4 Hz, 1H), 7.18 (d, J = 8.3 Hz, 1H), 7.00 (s, 1H), 4.62 (t, J = 6.9 Hz, 2H), 4.00-3.98 (m, 2H), 3.94 (s, 3H), 3.90-3.87 (m, 2H), 3.04 (t, J = 6.8 Hz, 2H), 2.27 (s, 3H), 2.24-2.18 (m, 2H), 2.08 (s, 3H); LC-MS: m/z 526.2 (M + H)⁺. |
| 90 | 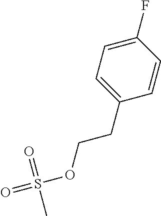 | 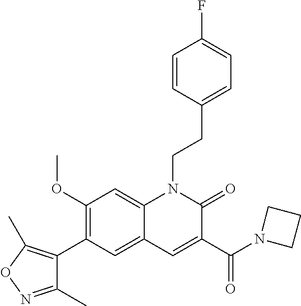 [SMILES: COc1cc2c(cc1-c1c(C)noc1C)cc(C(=O)N1CCC1)c(=O)n2CCc1ccc(F)cc1] | ¹H NMR (400 MHz, DMSO-d₆): δ 8.07 (s, 1H), 7.70 (s, 1H), 7.32-7.28 (m, 2H), 7.11 (t, J = 9.3 Hz, 2H), 7.01 (s, 1H), 4.57 (t, J = 7.3 Hz, 2H), 3.99-3.96 (m, 4H), 3.95 (s, 3H), 3.00 (t, J = 7.3 Hz, 2H), 2.27 (s, 3H), 2.23-2.19 (m, 2H), 2.09 (s, 3H); LC-MS: m/z 476.0 (M + H)⁺. |
| 91 | 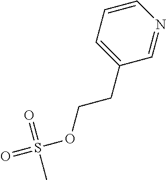 | 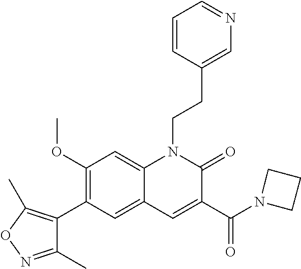 [SMILES: COc1cc2c(cc1-c1c(C)noc1C)cc(C(=O)N1CCC1)c(=O)n2CCc1cccnc1] | ¹H NMR (400 MHz, CDCl₃): δ 8.53 (s, 1H), 8.50 (d, J = 4.4 Hz, 1H), 8.03 (s, 1H), 7.64 (d, J = 7.9 Hz, 1H), 7.36 (s, 1H), 7.26-7.24 (m, 1H),6.72 (s, 1H), 4.56 (t, J = 8.3 Hz, 2H), 4.22 (t, J = 7.9 Hz, 2H), 4.11 (t, J = 7.9 Hz, 2H), 3.90 (s, 3H), 3.11 (t, J = 8.3 Hz, 2H), 2.35-2.28 (m, 2H), 2.30 (s, 3H), 2.15 (s, 3H); LC- MS: m/z 459.0 (M + H)⁺. |
| 92 | 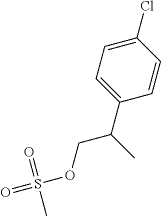 | 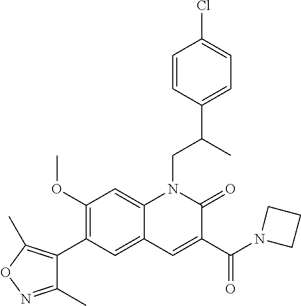 [SMILES: COc1cc2c(cc1-c1c(C)noc1C)cc(C(=O)N1CCC1)c(=O)n2CC(C)c1ccc(Cl)cc1] | ¹H NMR (400 MHz, CDCl₃): δ 8.02 (s, 1H), 7.32 (s, 1H), 7.26-7.16 (m, 4H), 6.59 (s, 1H), 4.70 (bs, 1H), 4.21 (t, J = 7.8 Hz, 3H), 4.07-3.89 (m, 2H), 3.83 (s, 3H), 3.39-3.34 (m, 1H), 2.32-2.17 (m, 2H), 2.29 (s, 3H), 2.14 (s, 3H), 1.41 (d, J = 6.8 Hz, 3H); LC-MS: m/z 506.2 (M + H)⁺. |
| 93 | 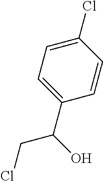 [SMILES: OC(CCl)c1ccc(Cl)cc1] | 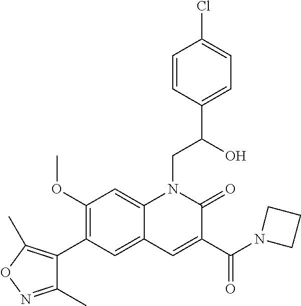 [SMILES: COc1cc2c(cc1-c1c(C)noc1C)cc(C(=O)N1CCC1)c(=O)n2CC(O)c1ccc(Cl)cc1] | ¹H NMR (400 MHz, DMSO-d₆): δ 8.06 (s, 1H), 7.67 (s, 1H), 7.42-7.36 (m, 4H), 7.18 (s, 1H), 5.80 (bs, 1H), 5.02-4.99 (m, 1H), 4.51-4.48 (m, 2H), 4.00-3.94 (m. 4H), 3.91 (s, 3H), 2.27 (s, 3H), 2.25-2.19 (m, 2H), 2.09 (s, 3H); LC- MS: m/z 508.2 (M + H)⁺. |
| 94 | 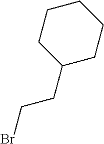 | 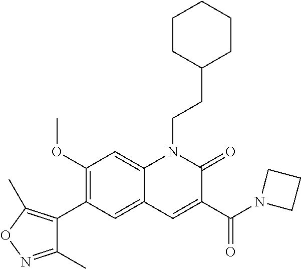 [SMILES: COc1cc2c(cc1-c1c(C)noc1C)cc(C(=O)N1CCC1)c(=O)n2CCC1CCCCC1] | ¹H NMR (400 MHz, DMSO-d₆): δ 8.06 (s, 1H), 7.72 (s, 1H), 6.99 (s, 1H), 4.36 (t, J = 7.3 Hz, 2H), 4.04-3.97 (m, 3H), 3.96 (s, 3H), 2.45-2.40 (m, 1H), 2.28 (s, 3H), 2.26-2.20 (m, 2H), 2.10 (s, 3H), 1.87-1.84 (m, 2H), 1.71-1.53 (m, 5H), 1.41-1.40 (m, 1H), 1.25-1.20 (m, 3H), 1.17-1.0 (m, 2H); ES-MS: m/z 464.2 (M + H)⁺. |
| 95 | 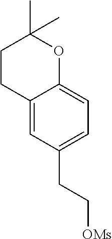 | 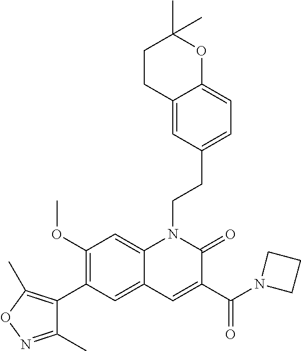 [SMILES: COc1cc2c(cc1-c1c(C)noc1C)cc(C(=O)N1CCC1)c(=O)n2CCc1ccc2c(c1)CCC(C)(C)O2] | ¹H NMR (400 MHz, DMSO-d₆): δ 8.06 (s, 1H), 7.69 (s, 1H), 6.96-6.92 (m, 3H), 6.59 (d, J = 8.3 Hz, 1H), 4.50 (t, J = 7.3 Hz, 2H), 4.01-3.95 (m, 4H), 3.92 (s, 3H), 2.87 (t, J = 8.3 Hz, 2H), 2.67-2.62 (m, 2H), 2.27 (s, 3H), 2.21 (t, J = 7.8 Hz, 2H), 2.08 (s, 3H), 1.70 (t, J = 6.8 Hz, 2H), 1.22 (s, 6H); LCMS: m/z 542.3 (M + H)⁺. |
| 96 | 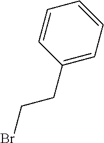 | 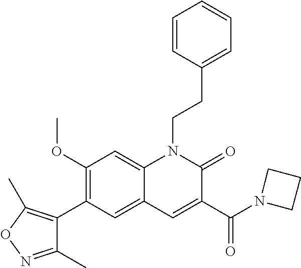 [SMILES: COc1cc2c(cc1-c1c(C)noc1C)cc(C(=O)N1CCC1)c(=O)n2CCc1ccccc1] | ¹H NMR (400 MHz, DMSO-d₆): δ 8.07 (s, 1H), 7.70 (s, 1H), 7.30 (d, J = 3.9 Hz, 4H), 7.22-7.20 (m, 1H), 7.01 (s, 1H), 4.57 (t, J = 7.3 Hz, 2H), 4.01-3.96 (m, 4H), 3.94 (s, 3H), 3.0 (t, J = 7.8 Hz, 2H), 2.27 (s, 3H), 2.21 (t, J = 7.8 Hz, 2H), 2.09 (s, 3H); LC-MS: m/z 458.2 (M + H)⁺. |
| 97 | 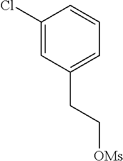 [SMILES: CS(=O)(=O)OCCc1cccc(Cl)c1] | 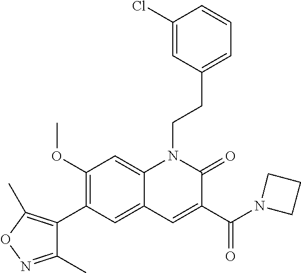 [SMILES: COc1cc2c(cc1-c1c(C)noc1C)cc(C(=O)N1CCC1)c(=O)n2CCc1cccc(Cl)c1] | ¹H NMR (400 MHz, DMSO-d₆): δ 8.21 (s, 1H), 7.80 (s, 1H), 7.50 (s, 1H), 7.40- 7.38 (m, 2H), 7.33-7.30 (m, 2H), 4.69 (t, J = 6.4 Hz, 2H), 3.99 (t, J = 7.3 Hz, 2H), 3.92 (s, 3H), 3.66 (t, J = 7.4 Hz, 2H), 3.15 (t, J = 6.4 Hz, 2H), 2.29 (s, 3H), 2.15-2.11 (m, 2H), 2.10 (s, 3H); LC-MS: m/z 492.2 (M + H)⁺. |

### Example-XVII: 6-(3,5-Dimethylisoxazol-4-yl)-N-(4-hydroxy-3,5-dimethylphenyl)-7-methoxy-N-methyl-2-oxo-1(pyridin-2-ylmethyl) 1,2-dihydroquinoline-3-carboxamide (Compound-98)

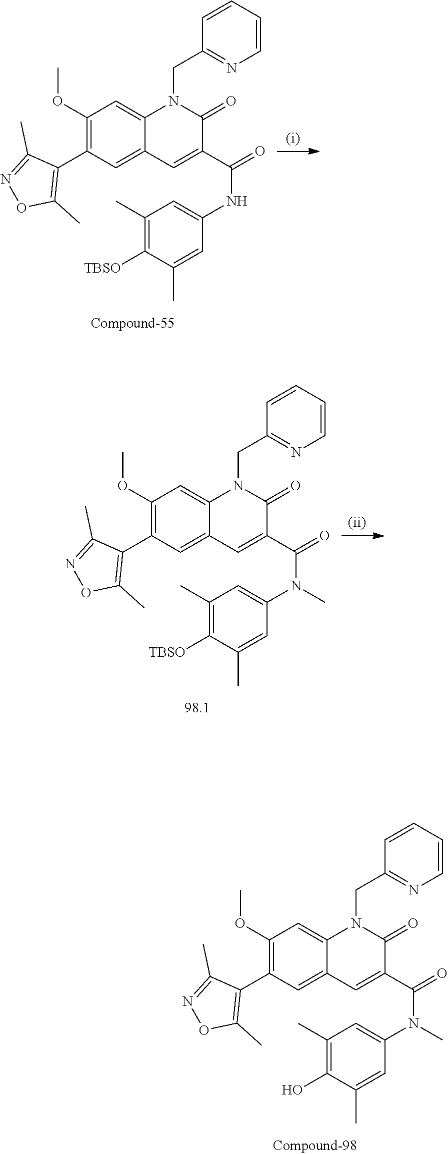 [SMILES: COc1cc2c(cc1-c1c(C)noc1C)cc(C(=O)Nc1cc(C)c(O[Si](C)(C)C(C)(C)C)c(C)c1)c(=O)n2Cc1ccccn1 ; COc1cc2c(cc1-c1c(C)noc1C)cc(C(=O)N(C)c1cc(C)c(O[Si](C)(C)C(C)(C)C)c(C)c1)c(=O)n2Cc1ccccn1 ; COc1cc2c(cc1-c1c(C)noc1C)cc(C(=O)N(C)c1cc(C)c(O)c(C)c1)c(=O)n2Cc1ccccn1]

### Step-i: N-(4-((tert-butyldimethylsilyl)oxy)-3,5-dimethylphenyl)-6-(3,5-dimethylisoxazol-4-yl)-7-methoxy-N-methyl-2-oxo-1-(pyridin-2-ylmethyl)-1,2-dihydroquinoline-3-carboxamide (98.1)

To a cooled solution of N-(4-((tert-butyldimethylsilyl)oxy)-3,5-dimethylphenyl)-6-(3,5-dimethylisoxazol-4-yl)-7-methoxy-2-oxo-1-(pyridin-2-ylmethyl)-1,2-dihydroquinoline-3-carboxamide (TBS protected Compound-55 from Example-IV) (0.1 g, 0.156 mmol) in THF (20 mL) were added sodium hydride (60%) (0.007 g, 0.187 mmol) and methyl iodide (0.05 mL, 0.78 mmol) followed by stirring at RT for 6 h. The mixture was quenched with ice water and extracted with EtOAc (50 mL×2). The combined organic layers were washed with water (50 mL) and brine (50 mL), dried over sodium sulphate and concentrated under reduced pressure to afford the title compound (0.07 g). ¹H NMR (400 MHz, CDCl3) δ 8.52 (d, J=4.4 Hz, 1H), 7.59-7.50 (m, 2H), 7.18-7.15 (m, 2H), 7.08 (s, 1H), 6.90 (s, 2H), 6.95-6.90 (m, 1H), 5.60-5.55 (m, 2H), 3.76 (s, 3H), 3.45 (s, 3H), 2.23 (s, 3H), 2.09 (s, 3H), 2.06 (s, 6H), 0.92 (s, 9H), 0.12 (s, 6H); LC-MS: m/z 653.2 (M+H)⁺.

### Step-ii: 6-(3,5-dimethylisoxazol-4-yl)-N-(4-hydroxy-3,5-dimethylphenyl)-7-methoxy-N-methyl-2-oxo-1-(pyridin-2-ylmethyl)-1,2-dihydroquinoline-3-carboxamide (Compound-98)

To a cooled solution of step-(i) compound (98.1) (0.07 g, 0.107 mmol) in THF (3 mL) was added tetra butyl ammonium fluoride 1.0 M in THF (0.16 ml) followed by stirring at RT for 2 h. The mixture was quenched with saturated NH₄Cl and extracted with EtOAc (50 mL), washed with water (50 mL), dried over sodium sulphate and concentrated under reduced pressure. The residue was purified by column chromatography (60-120 silica gel and 2% MeOH in DCM as eluent) to afford the title compound (0.025 g). ¹H NMR (400 MHz, DMSO-d₆): δ 8.52 (d, J=4.9 Hz, 1H), 8.05 (s, 1H), 7.80 (bs, 1H), 7.69-7.60 (m, 1H), 7.52 (s, 1H), 7.30 (t, J=7.5 Hz, 1H), 7.07 (s, 1H), 6.85 (s, 3H), 5.53 (s, 2H), 3.73 (s, 3H), 3.27 (s, 3H), 2.32 (s, 3H), 2.04 (s, 9H); LC-MS: m/z 539.2 (M+H)⁺.

### Example-XVIII: 6-(3,5-Dimethylisoxazol-4-yl)-3-((5-hydroxyindolin-1-yl)methyl)-7-methoxy-1-(pyridin-2-ylmethyl)quinolin-2(1H)-one (Compound-99)

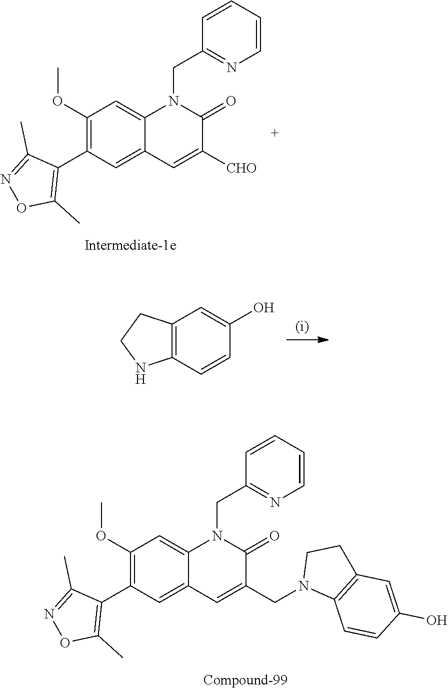 [SMILES: COc1cc2c(cc1-c1c(C)noc1C)cc(C=O)c(=O)n2Cc1ccccn1 ; Oc1ccc2c(c1)CCN2 ; COc1cc2c(cc1-c1c(C)noc1C)cc(CN1CCc3cc(O)ccc31)c(=O)n2Cc1ccccn1]

To a stirred solution of 6-(3,5-dimethylisoxazol-4-yl)-7-methoxy-2-oxo-1-(pyridin-2-ylmethyl)-1,2-dihydroquinoline-3-carbaldehyde (intermediate-1e) (0.2 g, 0.51 mmol) in titanium isopropoxide (5 mL) was added indolin-5-ol (0.1 g, 0.77 mmol) followed by stirring at RT for 16 h. After stirring, methanol (20 mL) was added to the mixture at 0° C. followed by NaCNBH₄ (0.16 g, 2.56 mmol). The mixture was stirred at RT for 2 h. The mixture was then quenched with ammonium hydroxide and the solids were filtered off. The filtrate was extracted with EtOAc (100 mL×2), washed with water (100 mL) and brine (100 mL), dried over sodium sulphate and concentrated under reduced pressure. The residue was purified to get the title compound as pale brown solid (0.016 g, 6%). ¹H NMR (400 MHz, CDCl3) δ 8.60 (d, J=4.8 Hz, 1H), 7.86 (s, 1H), 7.67 (t, J=7.8 Hz, 1H), 7.34 (d, J=7.8 Hz, 1H), 7.27 (s, 1H), 7.23-7.20 (m, 2H), 6.69 (s, 1H), 6.60-6.48 (m, 2H), 5.74 (s, 2H), 4.29 (s, 2H), 3.79 (s, 3H), 3.61-3.59 (m, 2H), 3.10-3.00 (m, 2H), 2.25 (s, 3H), 2.11 (s, 3H); LC-MS: m/z 509.3 (M+H)⁺.

### Example-XIX: 3-((1H-Tetrazol-5-yl)methyl)-6-(3,5-dimethylisoxazol-4-yl)-7-methoxy-1-(pyridin-2-ylmethyl)quinolin-2(1H)-one (Compound-100)

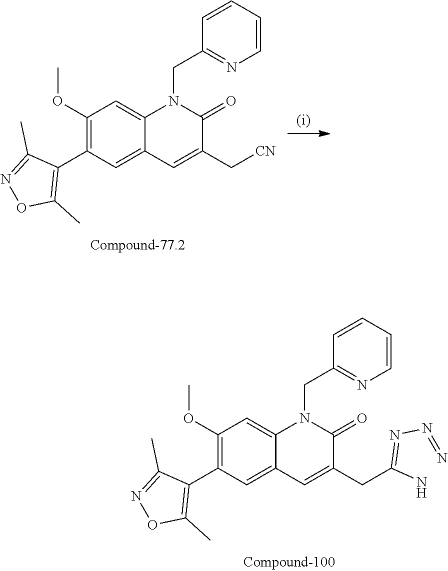 [SMILES: COc1cc2c(cc1-c1c(C)noc1C)cc(CC#N)c(=O)n2Cc1ccccn1 ; COc1cc2c(cc1-c1c(C)noc1C)cc(C/C(N)=N/N=N)c(=O)n2Cc1ccccn1]

To a solution of 2-(6-(3,5-dimethylisoxazol-4-yl)-7-methoxy-2-oxo-1-(pyridin-2-ylmethyl)-1,2-dihydroquinolin-3-yl)acetonitrile (compound-77.2) (0.05 g, 0.12 mmol) in DMF (1 mL) was added sodium azide (0.024 g, 0.37 mmol) and ammonium chloride (0.02 g, 0.37) portion wise followed by stirring at 120° C. for 16 h. The mixture was diluted with EtOAc (50 mL) and washed with water. The organic layer was dried over Na₂SO₄, concentrated under reduced pressure and column purified to obtain the title compound as greenish solid (0.015 g, 14%). ¹H NMR (400 MHz, DMSO-d₆) δ 16.5 (bs, 1H), 8.52 (d, J=4.4 Hz, 1H), 7.89 (s, 1H), 7.78-7.75 (m, 1H), 7.62 (s, 1H), 7.30 (d, J=7.4 Hz, 2H), 7.13 (s, 1H), 5.66 (s, 2H), 4.20 (s, 2H), 3.74 (s, 3H), 2.24 (s, 3H), 2.05 (s, 3H); LC-MS: m/z 444.2 (M+H).

### Example-XX: 2-(6-(3,5-Dimethylisoxazol-4-yl)-7-methoxy-2-oxo-1-(pyridin-2-yl-methyl)-1,2-dihydroquinolin-3-yl)-2-methylpropanoic Acid (Compound-101)

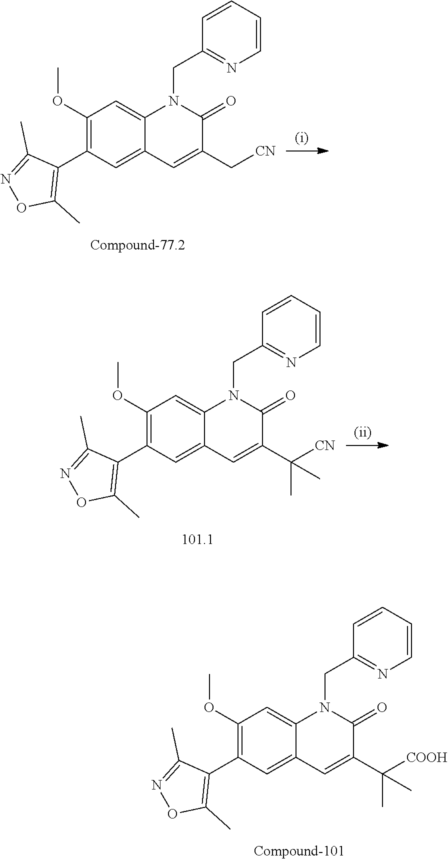 [SMILES: COc1cc2c(cc1-c1c(C)noc1C)cc(CC#N)c(=O)n2Cc1ccccn1 ; COc1cc2c(cc1-c1c(C)noc1C)cc(C(C)(C)C#N)c(=O)n2Cc1ccccn1 ; COc1cc2c(cc1-c1c(C)noc1C)cc(C(C)(C)C(=O)O)c(=O)n2Cc1ccccn1]

### Step-i: 2-(6-(3,5-Dimethylisoxazol-4-yl)-7-methoxy-2-oxo-1-(pyridin-2-yl-methyl)-1,2-dihydroquinolin-3-yl)-2-methylpropanenitrile (101.1)

To a cold suspension of sodium hydride (0.03 g, 0.75) in THF (4 mL) was added slowly 2-(6-(3,5-dimethylisoxazol-4-yl)-7-methoxy-2-oxo-1-(pyridin-2-ylmethyl)-1,2-dihydroquinolin-3-yl)acetonitrile (compound-77.2) (0.1 g, 0.25 mmol) in THF (1 mL) followed by stirring for 10 min. Methyl iodide (0.03 mL, 0.5 mmol) was then added followed by stirring at RT for 16 h. The mixture was quenched with ice water and extracted with EtOAc (50 mL). The organic layer was dried over Na₂SO₄, concentrated under reduced pressure and column purified to afford the title compound as white solid (0.06 g, 56%). ¹H NMR (400 MHz, DMSO-d₆) δ 8.52 (d, J=4.0 Hz, 1H), 8.04 (s, 1H), 7.82-7.77 (m, 1H), 7.71 (s, 1H), 7.34-7.29 (m, 2H), 7.13 (s, 1H), 5.71 (s, 2H), 3.75 (s, 3H), 2.25 (s, 3H), 2.06 (s, 3H), 1.75 (s, 6H); LC-MS: m/z 429.2 (M+H)⁺.

### Step-ii: 2-(6-(3,5-Dimethylisoxazol-4-yl)-7-methoxy-2-oxo-1-(pyridin-2-yl-methyl)-1,2-dihydro quinolin-3-yl)-2-methylpropanoic Acid (101)

The process of this step was adopted from step-iii of Example-XII. ¹H NMR (400 MHz, DMSO-d₆) δ 11.9 (bs, 1H), 8.54 (d, J=4.4 Hz, 1H), 7.85 (s, 1H), 7.77-7.73 (m, 1H), 7.64 (s, 1H), 7.31-7.28 (m, 1H), 7.23 (d, J=7.8 Hz, 1H), 7.11 (s, 1H), 5.65 (s, 2H), 3.74 (s, 3H), 2.25 (s, 3H), 2.05 (s, 3H), 1.46 (s, 6H); LC-MS: m/z 448.3 (M+H)⁺.

The below compound was prepared by a procedure similar to the one described in Example-XX by using appropriate starting compound (prepared according to Example-XII (step-ii)) and in the presence of suitable reagents and solvents at appropriate reaction conditions. The physiochemical characteristics of the compounds are also summarized.

| | | | Characterization Data |
|---|---|---|---|
| | | | ¹H NMR (400 MHz, DMSO- |
| No. | Starting Compound | Obtained Compound | d₆)/LC-MS |
| 102 | 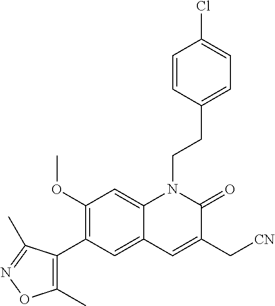 | 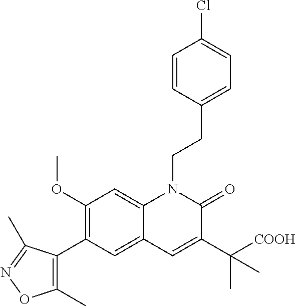 [SMILES: COc1cc2c(cc1-c1c(C)noc1C)cc(C(C)(C)C(=O)O)c(=O)n2CCc1ccc(Cl)cc1] | δ 8.52 (d, J = 4.9 Hz, 1H), 8.09 (s, 1H), 7.82-7.77 (m, 2H), 7.34- 7.29 (m, 2H), 7.16 (s, 1H), 5.72 (s, 2H), 4.92 (q, J = 9.8 Hz, 1H), 3.77 (s, 3H), 3.58 (t, J = 4.9 Hz, 4H), 2.74-2.67 (m, 2H), 2.59- 2.53 (m, 2H), 2.26 (s, 3H), 2.07 (s, 3H); LC-MS: m/z 529.2 (M + H)⁺. |
| | Step-v intermediate | | |
| | of Compound-78 | | |

### Example-XXI: 3-(Azetidine-1-carbonyl)-1-(2-(4-chlorophenyl)-2-oxoethyl)-6-(3,5-dimethylisoxazol-4-yl)-7-methoxyquinolin-2(1H)-one (Compound-103)

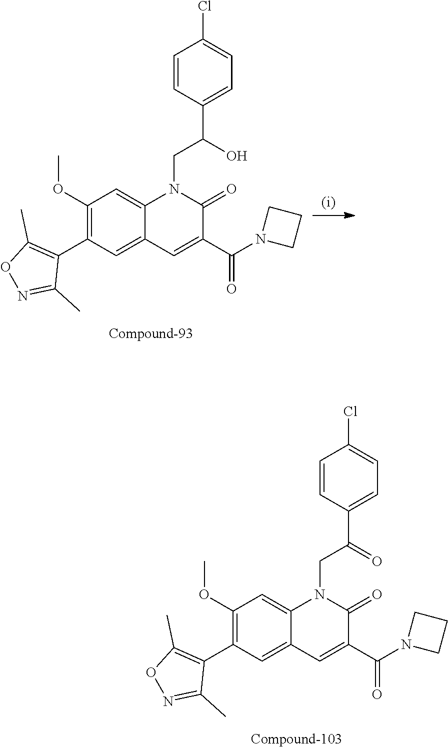 [SMILES: COc1cc2c(cc1-c1c(C)noc1C)cc(C(=O)N1CCC1)c(=O)n2CC(O)c1ccc(Cl)cc1 ; COc1cc2c(cc1-c1c(C)noc1C)cc(C(=O)N1CCC1)c(=O)n2CC(=O)c1ccc(Cl)cc1]

To a solution of 3-(azetidine-1-carbonyl)-1-(2-(4-chlorophenyl)-2-hydroxyethyl)-6-(3,5-dimethylisoxazol-4-yl)-7-methoxyquinolin-2(1H)-one (0.2 g, 0.39 mmol) in 1,4-dioxane (5 mL) was added manganese dioxide (0.1 g, 1.18 mmol) followed by stirring at 110° C. for 3 h. The mixture cooled to RT, filtered on celite bed and washed with EtOAc (50 mL). The combined organic layer was concentrated under reduced pressure and purified by combiflash to afford the title compound as white solid (0.015 g, 7%). ¹H NMR (400 MHz, DMSO-d₆): δ 8.18 (d, J=8.4 Hz, 2H), 8.17 (s, 1H), 7.77 (s, 1H), 7.72 (d, J=8.8 Hz, 2H), 7.02 (s, 1H), 5.96 (s, 2H), 3.97 (t, J=7.8 Hz, 4H), 3.81 (s, 3H), 2.28 (s, 3H), 2.23-2.16 (m, 2H), 2.09 (s, 3H); LC-MS: m/z 506.2 (M+H).

### Example-XXII: 6-(3,5-Dimethylisoxazol-4-yl)-N-(4,6-dimethylpyridin-2-yl)-7-methoxy-2-oxo-1-(pyridin-2-ylmethyl)-1,2-dihydroquinoline-3-carboxamide Hydrochloride (Compound-104)

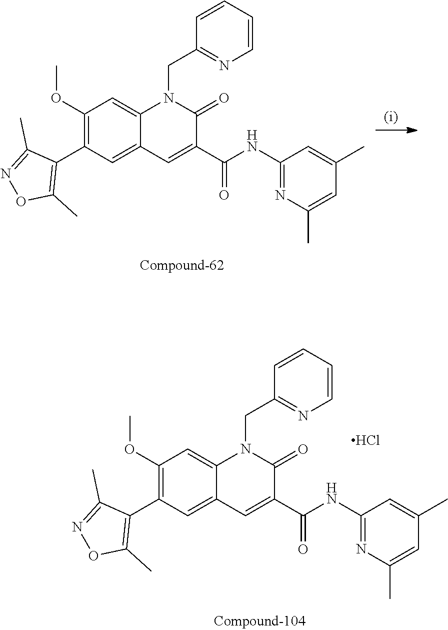 [SMILES: COc1cc2c(cc1-c1c(C)noc1C)cc(C(=O)Nc1cc(C)cc(C)n1)c(=O)n2Cc1ccccn1 ; COc1cc2c(cc1-c1c(C)noc1C)cc(C(=O)Nc1cc(C)cc(C)n1)c(=O)n2Cc1ccccn1 ; Cl]

To a solution of 6-(3,5-dimethylisoxazol-4-yl)-N-(4,6-dimethylpyridin-2-yl)-7-methoxy-2-oxo-1-(pyridin-2-ylmethyl)-1,2-dihydroquinoline-3-carboxamide (0.08 g, 0.16 mmol) in methanol (4 mL) was added 6 N HCl (1.5 mL) followed by stirring at RT for 3 h. The mixture was concentrated under reduced pressure to afford the title compound as yellow solid (0.06 g, 75%). ¹H NMR (400 MHz, DMSO-d₆): δ 12.45 (s, 1H), 9.07 (s, 1H), 8.64 (d, J=4.9 Hz, 1H), 8.10 (s, 1H), 8.09 (s, 1H), 8.02 (t, J=7.4 Hz, 1H), 7.59 (d, J=7.8 Hz, 1H), 7.53 (t, J=6.4 Hz, 1H), 7.27 (s, 1H), 7.05 (s, 1H), 5.94 (s, 2H), 3.87 (s, 3H), 2.44 (s, 3H), 2.39 (s, 3H), 2.29 (s, 3H), 2.10 (s, 3H); LCMS: m/z 510.3 (M+H)⁺.

### Example-XXIII: 3-(6-Aminopyridin-3-yl)-6-(3,5-dimethylisoxazol-4-yl)-7-methoxy-1-(pyridin-2-ylmethyl)quinolin-2(1H)-one (Compound 105)

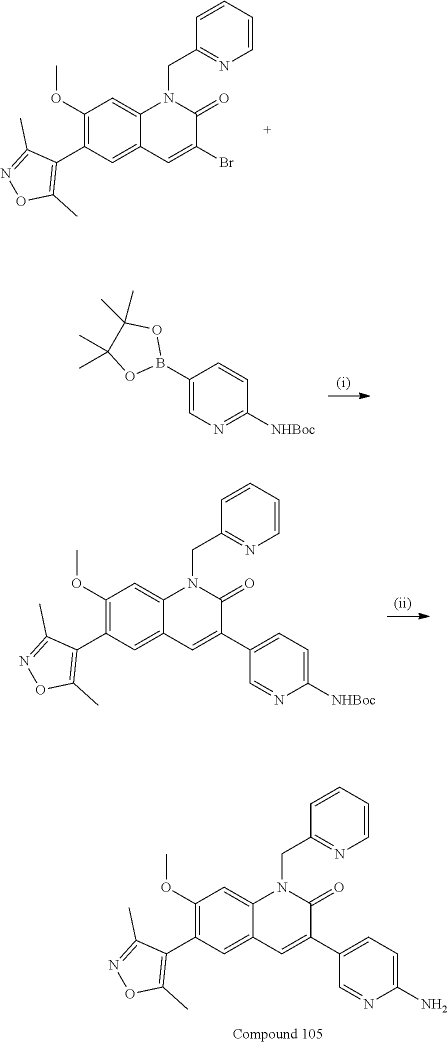 [SMILES: COc1cc2c(cc1-c1c(C)noc1C)cc(Br)c(=O)n2Cc1ccccn1 ; CC(C)(C)OC(=O)Nc1ccc(B2OC(C)(C)C(C)(C)O2)cn1 ; COc1cc2c(cc1-c1c(C)noc1C)cc(-c1ccc(NC(=O)OC(C)(C)C)nc1)c(=O)n2Cc1ccccn1 ; COc1cc2c(cc1-c1c(C)noc1C)cc(-c1ccc(N)nc1)c(=O)n2Cc1ccccn1]

### Step-i: tert-Butyl (5-(6-(3,5-dimethylisoxazol-4-yl)-7-methoxy-2-oxo-1-(pyridin-2-ylmethyl)-1,2-dihydroquinolin-3-yl)pyridin-2-yl)carbamate

To a solution of 3-bromo-6-(3,5-dimethylisoxazol-4-yl)-7-methoxy-1-(pyridin-2-ylmethyl)quinolin-2(1H)-one (0.05 g, 0.11 mmol) in 1,2-DME (8 mL) and H₂O (2 mL) were added tert-butyl (5-(4,4,5,5-tetramethyl-1,3,2-dioxaborolan-2-yl)pyridin-2-yl)-carbamate (0.054 g, 0.17 mmol) and sodium carbonate (0.04 g, 0.34 mmol). The mixture was degassed with nitrogen purging for 20 min. Then tetrakis triphenylphosphine palladium (0.013 g, 0.011 mmol) was added followed by heating at 90° C. for 16 h. The mixture was diluted with EtOAc (50 mL), washed with water (50 mL) and brine (50 mL), dried over sodium sulphate and concentrated under reduced pressure. The residue was purified by combi flash to afford the title compound as a pale yellow solid (0.05 g); LC-MS: m/z 554.3 (M+H).

### Step-ii: 3-(6-Aminopyridin-3-yl)-6-(3,5-dimethylisoxazol-4-yl)-7-methoxy-1-(pyridin-2-ylmethyl)quinolin-2(1H)-one

To a solution of tert-butyl (5-(6-(3,5-dimethylisoxazol-4-yl)-7-methoxy-2-oxo-1-(pyridin-2-ylmethyl)-1,2-dihydroquinolin-3-yl)pyridin-2-yl)carbamate (0.05 g, 0.09 mmol) in DCM (5 mL) was added TFA (0.07 mL, 0.9 mmol) followed by stirring at RT for 3 h. The mixture was concentrated and the residue was diluted with EtOAc, washed with aqueous NaHCO₃, dried over sodium sulphate, concentrated under reduced pressure and purified by combi flash to afford title compound as off white solid (0.02 g, 50%). ¹H NMR (400 MHz, DMSO-d₆) δ 8.52 (d, J=3.9 Hz, 1H), 8.34 (d, J=1.9 Hz, 1H), 8.04 (s, 1H), 7.83-7.76 (m, 2H), 7.64 (s, 1H), 7.35 (d, J=7.9 Hz, 1H), 7.32-7.29 (m, 1H), 7.12 (s, 1H), 6.49 (d, J=8.8 Hz, 1H), 6.09 (s, 2H), 5.72 (s, 2H), 3.75 (s, 3H), 2.26 (s, 3H), 2.07 (s, 3H); LC-MS: m/z 454.2 (M+H).

### Example-XXIV: N-(6-amino-5-methylpyridin-3-yl)-6-(3,5-dimethylisoxazol-4-yl)-7-methoxy-2-oxo-1-(pyridin-2-ylmethyl)-1,2-dihydroquinoline-3-carboxamide (Compound 106)

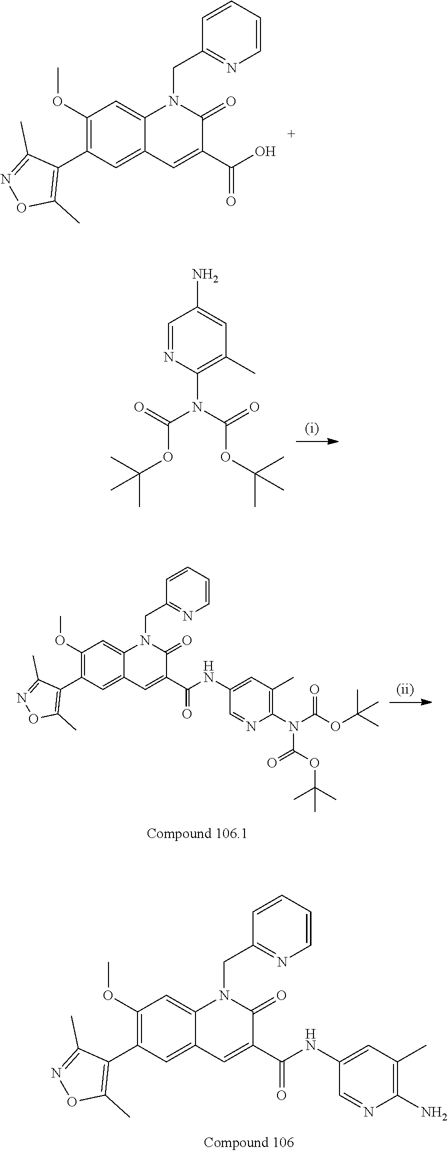 [SMILES: COc1cc2c(cc1-c1c(C)noc1C)cc(C(=O)O)c(=O)n2Cc1ccccn1 ; Cc1cc(N)cnc1N(C(=O)OC(C)(C)C)C(=O)OC(C)(C)C ; COc1cc2c(cc1-c1c(C)noc1C)cc(C(=O)Nc1cnc(N(C(=O)OC(C)(C)C)C(=O)OC(C)(C)C)c(C)c1)c(=O)n2Cc1ccccn1 ; COc1cc2c(cc1-c1c(C)noc1C)cc(C(=O)Nc1cnc(N)c(C)c1)c(=O)n2Cc1ccccn1]

### Step-i: Compound

To a solution of 6-(3,5-dimethylisoxazol-4-yl)-7-methoxy-2-oxo-1-(pyridin-2-yl-methyl)-1,2-dihydroquinoline-3-carboxylic acid (0.15 g, 0.37 mmol) in DMF (4 mL) were added Intermediate-23 (0.18 g, 0.55 mmol), HOBt (0.15 g, 1.11 mmol), EDC.HCl (0.21 g, 1.11 mmol) and triethyl amine (0.15 mL, 1.11 mmol) followed by stirring at RT for 16 h. The mixture was diluted with EtOAc (50 mL), washed with water (50 mL) and brine (50 mL), dried over sodium sulphate, concentrated under reduced pressure and purified by combi flash to afford the title compound as yellow solid (0.17 g, 65%); ¹H NMR (400 MHz, DMSO-d₆): δ 12.13 (s, 1H), 9.04 (s, 1H), 8.70 (d, J=2.5 Hz, 1H), 8.52 (d, J=4.9 Hz, 1H), 8.18 (d, J=2.4 Hz, 1H), 8.04 (s, 1H), 7.83-7.79 (m, 1H), 7.48 (d, J=7.8 Hz, 1H), 7.34-7.31 (m, 1H), 7.24 (s, 1H), 5.85 (s, 2H), 3.83 (s, 3H), 2.29 (s, 3H), 2.17 (s, 3H), 2.10 (s, 3H), 1.35 (s, 18H).

### Step-ii: N-(6-amino-5-methylpyridin-3-yl)-6-(3,5-dimethylisoxazol-4-yl)-7-methoxy-2-oxo-1-(pyridin-2-ylmethyl)-1,2-dihydroquinoline-3-carboxamide (Compound 106)

To a cold solution of Compound 106.1 (0.17 g, 0.24 mmol) in DCM (5 mL) was added TFA (0.5 mL) followed by stirring at RT for 4 h. The mixture was concentrated, diluted with DCM and washed with aqueous NaHCO₃. The organic layer was dried over sodium sulphate and concentrated. The residue was purified by combi flash to afford the title compound as yellow solid (0.045 g, 38%). ¹H NMR (400 MHz, DMSO-d₆): δ 11.62 (s, 1H), 8.97 (s, 1H), 8.51 (d, J=4.4 Hz, 1H), 8.19 (d, J=2.5 Hz, 1H), 8.01 (s, 1H), 7.83-7.78 (m, 1H), 7.64 (d, J=2.0 Hz, 1H), 7.46 (d, J=7.8 Hz, 1H), 7.33-7.30 (m, 1H), 7.22 (s, 1H), 5.83 (s, 2H), 5.65 (s, 2H), 3.81 (s, 3H), 2.28 (s, 3H), 2.09 (s, 3H), 2.07 (s, 3H); LC-MS: m/z 511.2 (M+H).

### Example-XXV: 6-(3,5-Dimethylisoxazol-4-yl)-3-((2,6-dimethylpyridin-4-yl)-amino)-7-methoxy-1-(pyridin-2-ylmethyl)quinolin-2(1H)-one (Compound 107)

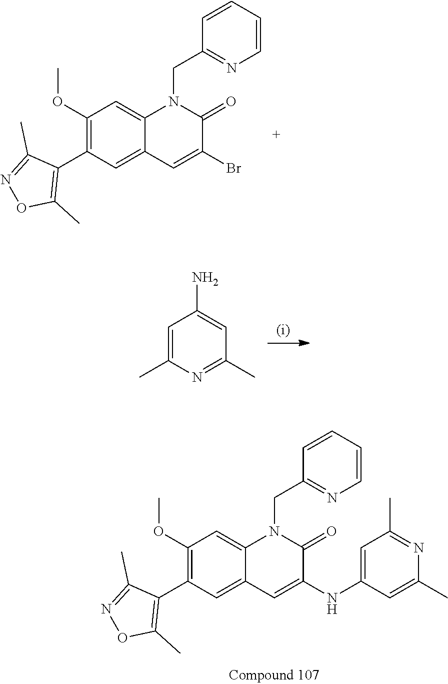

To a solution of 3-bromo-6-(3,5-dimethylisoxazol-4-yl)-7-methoxy-1-(pyridin-2-ylmethyl)quinolin-2(1H)-one (0.15 g, 0.34 mmol) in 1,4-dioxane (6 mL) were added 2,6-dimethylpyridin-4-amine (0.062 g, 0.51 mmol) and cesium carbonate (0.33 g, 1.02 mmol). The mixture was degassed with nitrogen purging for 15 min. Then tris(di-benzylideneacetone)di palladium (0) (0.031 g, 0.034 mmol) and xantphos (0.010 g, 0.017 mmol) were added followed by heating at 100° C. for 16 h. The mixture was diluted with EtOAc (50 mL), washed with water (50 mL) and brine (50 mL), dried over sodium sulphate, concentrated under reduced pressure and purified by combi flash to afford the title compound as pale yellow solid (0.05 g, 37%). ¹H NMR (400 MHz, DMSO-d₆) δ 8.51 (d, J=4.4 Hz, 1H), 8.32 (bs, 1H), 7.83 (s, 1H), 7.81-7.77 (m, 1H), 7.64 (s, 1H), 7.38 (d, J=7.8 Hz, 1H), 7.32-7.29 (m, 1H), 7.12 (s, 1H), 6.94 (s, 2H), 5.76 (s, 2H), 3.73 (s, 3H), 2.34 (s, 6H), 2.26 (s, 3H), 2.07 (s, 3H); LC-MS: m/z 482.3 (M+H).

### Example-XXVI: 3-(6-Amino-5-methylpyridin-3-yl)-6-(3,5-dimethylisoxazol-4-yl)-7-methoxy-1-(pyridin-2-ylmethyl)quinolin-2(1H)-one (Compound 108)

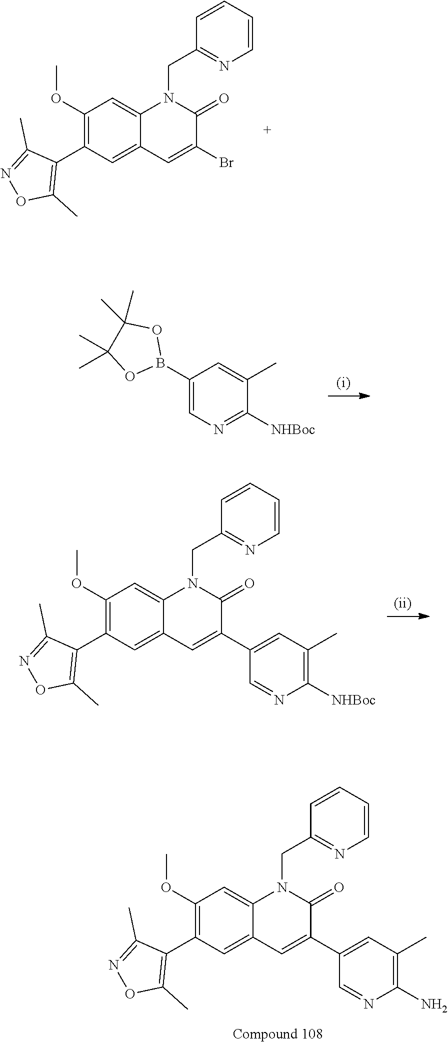 [SMILES: COc1cc2c(cc1-c1c(C)noc1C)cc(Br)c(=O)n2Cc1ccccn1 ; Cc1cc(B2OC(C)(C)C(C)(C)O2)cnc1NC(=O)OC(C)(C)C ; COc1cc2c(cc1-c1c(C)noc1C)cc(-c1cnc(NC(=O)OC(C)(C)C)c(C)c1)c(=O)n2Cc1ccccn1 ; COc1cc2c(cc1-c1c(C)noc1C)cc(-c1cnc(N)c(C)c1)c(=O)n2Cc1ccccn1]

### Step-i: tert-Butyl (5-(6-(3,5-dimethylisoxazol-4-yl)-7-methoxy-2-oxo-1-(pyridin-2-ylmethyl)-1,2-dihydroquinolin-3-yl)-3-methylpyridin-2-yl)carbamate

To a solution of 3-bromo-6-(3,5-dimethylisoxazol-4-yl)-7-methoxy-1-(pyridin-2-ylmethyl)quinolin-2(1H)-one (0.12 g, 0.27 mmol) in 1,4-dioxane (6 mL) and H₂O (2 mL) were added tert-butyl (3-methyl-5-(4,4,5,5-tetramethyl-1,3,2-dioxaborolan-2-yl)pyridin-2-yl)carbamate (0.13 g, 0.40 mmol) and sodium carbonate (0.086 g, 0.81 mmol) followed by degassing with nitrogen purging for 20 min. Then tetrakis triphenylphosphine palladium (0.031 g, 0.027 mmol) was added followed by heating at 90° C. for 16 h. The mixture was diluted with EtOAc (50 mL), washed with water (50 mL) and brine (50 mL), dried over sodium sulphate, concentrated under reduced pressure and purified by combi flash to afford the title compound as a pale yellow gummy mass (0.09 g, 58%). ¹H NMR (400 MHz, DMSO-d₆): δ 8.68 (d, J=1.5 Hz, 1H), 8.54 (d, J=4.4 Hz, 1H), 8.31 (s, 1H), 8.18 (s, 1H), 7.80-7.77 (m, 1H), 7.71 (s, 1H), 7.41 (d, J=7.4 Hz, 2H), 7.33-7.29 (m, 1H), 7.16 (s, 1H), 5.75 (s, 2H), 3.78 (s, 3H), 2.27 (s, 3H), 2.21 (s, 3H), 2.08 (s, 3H), 1.39 (s, 9H).

### Step-ii: 3-(6-Amino-5-methylpyridin-3-yl)-6-(3,5-dimethylisoxazol-4-yl)-7-methoxy-1-(pyridin-2-ylmethyl)quinolin-2(1H)-one

To a cold solution of tert-butyl (5-(6-(3,5-dimethylisoxazol-4-yl)-7-methoxy-2-oxo-1-(pyridin-2-ylmethyl)-1,2-dihydroquinolin-3-yl)-3-methylpyridin-2-yl)carbamate (0.09 g, 0.158 mmol) in DCM (5 mL) was added TFA (2 mL) followed by stirring at RT for 4 h. The mixture was concentrated and the residue was diluted with EtOAc, washed with aqueous NaHCO₃, dried over sodium sulphate, concentrated under reduced pressure and purified by combi flash to afford the title compound as white solid (0.02 g, 27%). ¹H NMR (400 MHz, DMSO-d₆): δ 8.53 (d, J=4.4 Hz, 1H), 8.24 (d, J=1.9 Hz, 1H), 8.04 (s, 1H), 7.79-7.75 (m, 1H), 7.69 (s, 1H), 7.64 (s, 1H), 7.35 (d, J=7.8 Hz, 1H), 7.31-7.28 (m, 1H), 7.12 (s, 1H), 5.89 (s, 2H), 5.71 (s, 2H), 3.75 (s, 3H), 2.26 (s, 3H), 2.10 (s, 3H), 2.07 (s, 3H); LC-MS: m/z 468.2 (M+H).

### Example-XXVII: 3-(Azetidine-1-carbonyl)-6-(3,5-dimethylisoxazol-4-yl)-7-methoxy-1-((3-methoxypyridin-2-yl)methyl)quinolin-2(1H)-one (Compound-109)

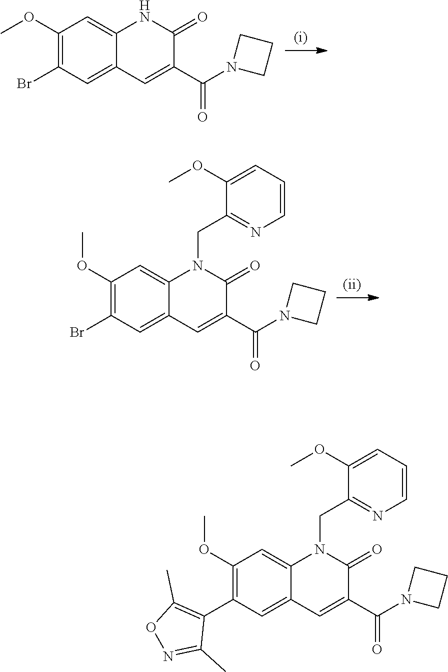 [SMILES: COc1cc2[nH]c(=O)c(C(=O)N3CCC3)cc2cc1Br ; COc1cc2c(cc1Br)cc(C(=O)N1CCC1)c(=O)n2Cc1ncccc1OC ; COc1cc2c(cc1-c1c(C)noc1C)cc(C(=O)N1CCC1)c(=O)n2Cc1ncccc1OC]

### Step-i: Synthesis of 3-(azetidine-1-carbonyl)-6-bromo-7-methoxy-1-((3-methoxypyridin-2-yl) methyl) quinolin-2(1H)-one

To a solution of 3-(azetidine-1-carbonyl)-6-bromo-7-methoxyquinolin-2(1H)-one (0.4 g, 1.18 mmol, Intermediate-18) in DMF (10 mL) were added potassium carbonate (0.49 g, 3.54 mmol) and 2-(chloromethyl)-3-methoxypyridine (0.19 g, 1.18 mmol) followed by heating to 60° C. for 16 h. The mixture was poured into ice water and extracted with EtOAc. The organic layer was dried over sodium sulphate and concentrated under reduced pressure. The residue was purified by column chromatography to afford the title compound as an off white solid (0.3 g, 55%). ¹H NMR (400 MHz, DMSO-d₆): δ 8.07 (d, J=8.3 Hz, 2H), 7.90-7.89 (m, 1H), 7.47 (dd, J=8.3 Hz, 1.0 Hz, 1H), 7.27-7.24 (m, 1H), 6.86 (s, 1H), 5.63 (s, 2H), 4.00-3.96 (m, 4H), 3.92 (s, 3H), 3.76 (s, 3H), 2.24-2.18 (m, 2H); LC-MS: m/z 460.1 (M+2H)²⁺.

### Step-ii: 3-(Azetidine-1-carbonyl)-6-(3,5-dimethylisoxazol-4-yl)-7-methoxy-1-((3-methoxypyridin-2-yl)methyl)quinolin-2(1H)-one

To a solution of 3-(azetidine-1-carbonyl)-6-bromo-7-methoxy-1-((3-methoxypyridin-2-yl)methyl)quinolin-2(1H)-one (0.3 g, 0.65 mmol) in 1,2-DME (12 mL) and H₂O (4 mL) were added 3,5-dimethylisoxazoleboronic acid (0.14 g, 0.97 mmol) and sodium carbonate (0.21 g, 1.95 mmol) followed by degassing with nitrogen purging for 20 min. Then tetrakis triphenylphosphine palladium (0.075 g, 0.065 mmol) was added followed by heating to 90° C. for 16 h. The mixture was diluted with EtOAc (50 mL), washed with water (50 mL) and brine (50 mL), dried over sodium sulphate and concentrated under reduced pressure. The residue was purified by column chromatography to afford the title compound as white solid (0.02 g, 6%). ¹H NMR (400 MHz, DMSO-d₆) δ 8.10 (s, 1H), 7.94-7.7.92 (m, 1H), 7.71 (s, 1H), 7.50-7.47 (m, 1H), 7.28-7.26 (m, 1H), 6.89 (s, 1H), 5.65 (s, 2H), 4.00-3.98 (m, 4H), 3.94 (s, 3H), 3.72 (s, 3H), 2.26 (s, 3H), 2.22-2.21 (m, 2H), 2.07 (s, 3H); LC-MS: m/z 475.2 (M+H)⁺.

### Example-XXVIII: 3-(6-Aminopyridin-3-yl)-6-(3,5-dimethylisoxazol-4-yl)-7-methoxy-1-((3-methoxypyridin-2-yl)methyl)quinolin-2(1H)-one (Compound-110)

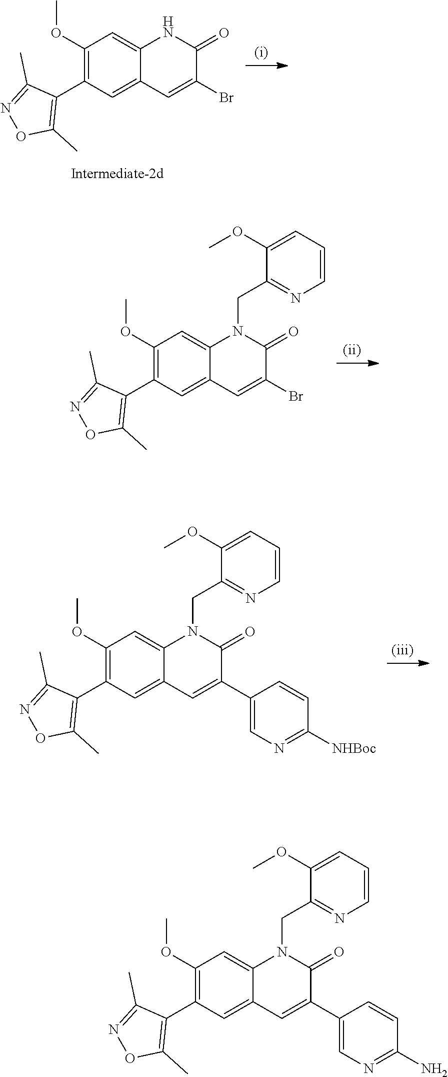 [SMILES: COc1cc2[nH]c(=O)c(Br)cc2cc1-c1c(C)noc1C ; COc1cc2c(cc1-c1c(C)noc1C)cc(Br)c(=O)n2Cc1ncccc1OC ; COc1cc2c(cc1-c1c(C)noc1C)cc(-c1ccc(NC(=O)OC(C)(C)C)nc1)c(=O)n2Cc1ncccc1OC ; COc1cc2c(cc1-c1c(C)noc1C)cc(-c1ccc(N)nc1)c(=O)n2Cc1ncccc1OC]

### Step-i: 3-Bromo-6-(3,5-dimethylisoxazol-4-yl)-7-methoxy-1-((3-methoxypyridin-2-yl)methyl)quinolin-2(1H)-one

The process of this step was adopted from step-i of Example-XXVII. ¹H NMR (400 MHz, DMSO-d₆): δ 8.47 (s, 1H), 7.93 (d, J=4.4 Hz, 1H), 7.63 (s, 1H), 7.50 (d, J=7.9 Hz, 1H), 7.30-7.27 (m, 1H), 6.84 (s, 1H), 5.69 (s, 2H), 3.95 (s, 3H), 3.70 (s, 3H), 2.26 (s, 3H), 2.06 (s, 3H); LC-MS: m/z 470.1 (M+H)⁺.

### Step-ii: tert-Butyl (5-(6-(3,5-dimethylisoxazol-4-yl)-7-methoxy-1-((3-methoxypyridin-2-yl)methyl)-2-oxo-1,2-dihydroquinolin-3-yl)pyridin-2-yl)carbamate

The process of this step was adopted from step-ii of Example-XXVII. ¹H NMR (400 MHz, DMSO-d6): δ 9.86 (s, 1H), 8.63 (d, J=1.9 Hz, 1H), 8.18 (s, 1H), 8.13-8.11 (m, 1H), 7.95 (d, J=3.9 Hz, 1H), 7.84 (d, J=8.8 Hz, 1H), 7.69-7.51 (m, 2H), 7.30-7.26 (m, 1H), 6.86 (s, 1H), 5.71 (s, 2H), 3.96 (s, 3H), 3.71 (s, 3H), 2.28 (s, 3H), 2.08 (s, 3H), 1.48 (s, 9H); LC-MS: m/z 584.3 (M+H)⁺.

### Step-iii: 3-(6-Aminopyridin-3-yl)-6-(3,5-dimethylisoxazol-4-yl)-7-methoxy-1-((3-methoxypyridin-2-yl)methyl)quinolin-2(1H)-one

To a solution of tert-butyl (5-(6-(3,5-dimethylisoxazol-4-yl)-7-methoxy-1-((3-methoxypyridin-2-yl)methyl)-2-oxo-1,2-dihydroquinolin-3-yl)pyridin-2-yl)carbamate (0.11 g, 0.19 mmol) in DCM (2 mL) was added TFA (0.5 mL) followed by stirring at RT for 3 h. The mixture was concentrated under reduced pressure, the residue was diluted with water, neutralized with aqueous NaHCO₃ and extracted with EtOAc. The organic layer was dried over sodium sulphate and concentrated under reduced pressure. The residue was purified by preparative TLC to afford the title compound as an off white solid (0.02 g, 22%). ¹H NMR (400 MHz, DMSO-d6): δ 8.32 (d, J=1.9 Hz, 1H), 8.01 (s, 1H), 7.94 (d, J=3.9 Hz, 1H), 7.80-7.77 (m, 1H), 7.62 (s, 1H), 7.49 (d, J=7.9 Hz, 1H), 7.29-7.26 (m, 1H), 6.83 (s, 1H), 6.47 (d, J=8.4 Hz, 1H), 6.83 (s, 2H), 5.69 (s, 2H), 3.96 (s, 3H), 3.69 (s, 3H), 2.27 (s, 3H), 2.08 (s, 3H). LC-MS: m/z 484.2 (M+H)⁺.

### Example-XXIX: 6-(3,5-Dimethylisoxazol-4-yl)-N-(4-hydroxy-3,5-dimethylphenyl)-7-methoxy-1-((3-methoxypyridin-2-yl)methyl)-2-oxo-1,2-dihydroquinoline-3-carboxamide (Compound-111)

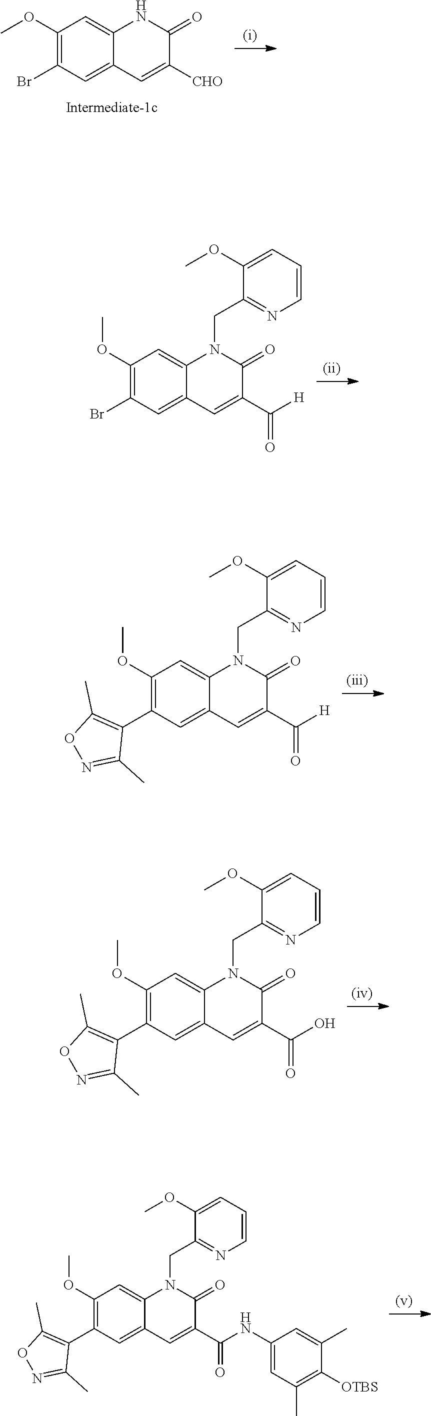 [SMILES: COc1cc2[nH]c(=O)c(C=O)cc2cc1Br ; COc1cc2c(cc1Br)cc(C=O)c(=O)n2Cc1ncccc1OC ; COc1cc2c(cc1-c1c(C)noc1C)cc(C=O)c(=O)n2Cc1ncccc1OC ; COc1cc2c(cc1-c1c(C)noc1C)cc(C(=O)O)c(=O)n2Cc1ncccc1OC ; COc1cc2c(cc1-c1c(C)noc1C)cc(C(=O)Nc1cc(C)c(O[Si](C)(C)C(C)(C)C)c(C)c1)c(=O)n2Cc1ncccc1OC]

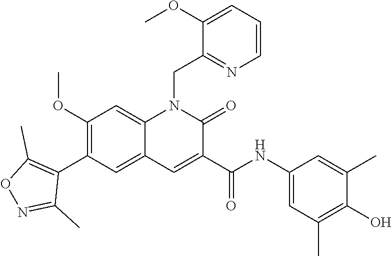 [SMILES: COc1cc2c(cc1-c1c(C)noc1C)cc(C(=O)Nc1cc(C)c(O)c(C)c1)c(=O)n2Cc1ncccc1OC]

### Step-i: 6-Bromo-7-methoxy-1-((3-methoxypyridin-2-yl)methyl)-2-oxo-1,2-dihydroquinoline-3-carbaldehyde

The process of this step was adopted from step-i of Example-XXVII with appropriate changes. ¹H NMR (400 MHz, DMSO-d₆) δ 10.27 (s, 1H), 8.69 (s, 1H), 8.44 (s, 1H), 8.13 (d, J=4.9 Hz, 1H), 7.54-7.52 (m, 1H), 7.41-7.37 (m, 1H), 7.31 (s, 1H), 5.71 (s, 2H), 4.03 (s, 3H), 3.88 (s, 3H); LC-MS: m/z 405.0 (M+2H)²⁺.

### Step-ii: 6-(3,5-Dimethylisoxazol-4-yl)-7-methoxy-1-((3-methoxypyridin-2-yl)-methyl)-2-oxo-1,2-dihydroquinoline-3-carbaldehyde

The process of this step was adopted from step-ii of Example-XXVII with appropriate changes. ¹H NMR (400 MHz, DMSO-d₆) δ 10.30 (s, 1H), 8.71 (s, 1H), 8.13 (d, J=4.4 Hz, 1H), 8.03 (s, 1H), 7.65-7.54 (m, 2H), 7.32 (s, 1H), 5.74 (s, 2H), 3.96 (s, 3H), 3.89 (s, 3H), 2.31 (s, 3H), 2.12 (s, 3H); LC-MS: m/z 420.3 (M+H)⁺.

### Step-iii: 6-(3,5-Dimethylisoxazol-4-yl)-7-methoxy-1-((3-methoxypyridin-2-yl)-methyl)-2-oxo-1,2-dihydroquinoline-3-carboxylic Acid

To a cold solution of 6-(3,5-dimethylisoxazol-4-yl)-7-methoxy-1-((3-methoxypyridin-2-yl)methyl)-2-oxo-1,2-dihydroquinoline-3-carbaldehyde (1.0 g, 2.38 mmol) in mixture of acetonitrile (10 mL) and H₂O (5 mL) were added sodium di hydrogen phosphate (1.0 g, 8.33 mmol), hydrogen peroxide 30% (0.6 mL) and sodium chlorite (0.43 g, 4.76 mmol) portion wise. The mixture was stirred at RT for 4 h. The mixture was diluted with cold water and extracted with DCM. The organic layer was dried over sodium sulphate and concentrated under reduced pressure. The residue was purified by combi-flash to afford the title compound as pink solid (0.3 g, 29%). ¹H NMR (400 MHz, DMSO-d₆) δ 13.10-13.0 (bs, 1H), 8.70 (s, 1H), 8.10 (d, J=3.9 Hz, 1H), 7.93 (s, 1H), 7.52 (d, J=8.3 Hz, 1H), 7.40-7.37 (m, 1H), 7.28 (s, 1H), 5.68 (s, 2H), 3.94 (s, 3H), 3.89 (s, 3H), 2.31 (s, 3H), 2.10 (s, 3H); LC-MS: m/z 436.1 (M+H)⁺.

### Step-iv: N-(4-((tert-butyldimethylsilyl)oxy)-3,5-dimethylphenyl)-6-(3,5-dimethylisoxazol-4-yl)-7-methoxy-1-((3-methoxypyridin-2-yl)methyl)-2-oxo-1,2-dihydroquinoline-3-carboxamide

To a solution of 6-(3,5-dimethylisoxazol-4-yl)-7-methoxy-1-((3-methoxypyridin-2-yl)methyl)-2-oxo-1,2-dihydroquinoline-3-carboxylic acid (0.14 g, 0.32 mmol) in DMF (5 mL) were added 4-((tert-butyldimethylsilyl)oxy)-3,5-dimethylaniline (0.1 g, 0.38 mmol), triethyl amine (0.13 mL, 0.96 mmol) and PyBOP (0.25 g, 0.48 mmol) followed by stirring at RT for 16 h. The mixture was diluted with EtOAc (50 mL), washed with water (50 mL) and brine (50 mL), dried over sodium sulphate and concentrated under reduced pressure. The residue was purified by combi-flash to afford the title compound as pale brown solid (0.08 g, 37%). ¹H NMR (400 MHz, DMSO-d₆) δ 10.83 (s, 1H), 8.91 (s, 1H), 8.20 (d, J=4.4 Hz, 1H), 8.03 (s, 1H), 7.59 (d, J=8.3 Hz, 1H), 7.49-7.46 (m, 1H), 7.41-7.40 (m, 3H), 5.86 (s, 2H), 3.97 (s, 3H), 3.94 (s, 3H), 2.32 (s, 3H), 2.19 (s, 6H), 2.13 (s, 3H), 1.01 (s, 9H), 0.19 (s, 6H); LC-MS: m/z 669.3 (M+H)⁺.

### Step-v: 6-(3,5-Dimethylisoxazol-4-yl)-N-(4-hydroxy-3,5-dimethylphenyl)-7-methoxy-1-((3-methoxypyridin-2-yl)methyl)-2-oxo-1,2-dihydroquinoline-3-carboxamide

To a cooled solution of N-(4-((tert-butyldimethylsilyl)oxy)-3,5-dimethylphenyl)-6-(3,5-dimethylisoxazol-4-yl)-7-methoxy-1-((3-methoxypyridin-2-yl)methyl)-2-oxo-1,2-dihydroquinoline-3-carboxamide (0.08 g, 0.12 mmol) in THF (3 mL) was added tetra butyl ammonium fluoride 1.0 M in THF (0.8 mL) followed by stirring at RT for 16 h. The reaction mixture was quenched with saturated NH₄Cl, extracted with EtOAc (50 mL), washed with water (50 mL), dried over sodium sulphate and concentrated under reduced pressure. The residue was purified by column chromatography to afford the title compound as an off white solid (0.03 g, 45%). ¹H NMR (400 MHz, DMSO-d₆): δ10.76 (s, 1H), 8.92 (s, 1H), 8.21 (d, J=4.4 Hz, 1H), 8.16 (s, 1H), 8.03 (s, 1H), 7.60 (d, J=8.3 Hz, 1H), 7.50-7.47 (m, 1H), 7.40 (s, 1H), 7.34 (s, 2H), 5.86 (s, 2H), 3.97 (s, 3H), 3.94 (s, 3H), 2.32 (s, 3H), 2.19 (s, 6H), 2.13 (s, 3H); LC-MS: m/z 555.3 (M+H)⁺.

### Example-XXX: N-(6-amino-5-methylpyridin-3-yl)-6-(3,5-dimethylisoxazol-4-yl)-7-methoxy-1-((3-methoxypyridin-2-yl)methyl)-2-oxo-1,2-dihydroquinoline-3-carboxamide (Compound-112)

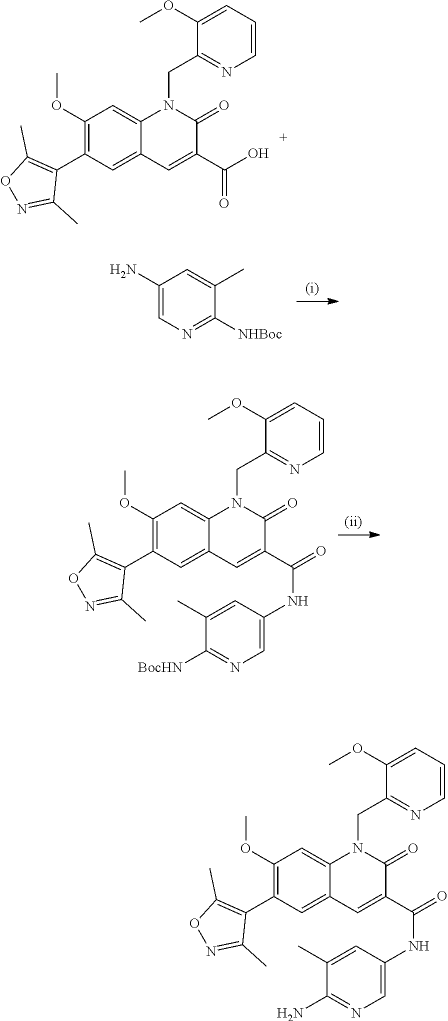 [SMILES: COc1cc2c(cc1-c1c(C)noc1C)cc(C(=O)O)c(=O)n2Cc1ncccc1OC ; Cc1cc(N)cnc1NC(=O)OC(C)(C)C ; COc1cc2c(cc1-c1c(C)noc1C)cc(C(=O)Nc1cnc(NC(=O)OC(C)(C)C)c(C)c1)c(=O)n2Cc1ncccc1OC ; COc1cc2c(cc1-c1c(C)noc1C)cc(C(=O)Nc1cnc(N)c(C)c1)c(=O)n2Cc1ncccc1OC]

### Step i: Synthesis of tert-butyl (5-(6-(3,5-dimethylisoxazol-4-yl)-7-methoxy-1-((3-methoxypyridin-2-yl)methyl)-2-oxo-1,2-dihydroquinoline-3-carboxamido)-3-methyl pyridin-2-yl)carbamate

To a cold solution of 6-(3,5-dimethylisoxazol-4-yl)-7-methoxy-1-((3-methoxypyridin-2-yl)methyl)-2-oxo-1,2-dihydroquinoline-3-carboxylic acid (0.15 g, 0.34 mmol) in DCM (3 mL) were added HATU (0.26 g, 0.68 mmol), tert-butyl (5-amino-3-methylpyridin-2-yl)carbamate (0.09 g, 0.41 mmol, Intermediate-24) and pyridine (0.08 mL, 1.02 mmol) followed by stirring at RT for 16 h. The mixture was diluted with DCM (50 mL), washed with water (50 mL) and brine (50 mL), dried over sodium sulphate and concentrated under reduced pressure. The residue was purified by combi-flash to afford the title compound as off white solid (0.16 g, 72%). ¹H NMR (400 MHz, DMSO-d₆) δ 11.24 (s, 1H), 9.12 (s, 1H), 8.95 (s, 1H), 8.61 (d, J=1.4 Hz, 1H), 8.17 (d, J=4.9 Hz, 1H), 8.07-8.04 (m, 2H), 7.64-7.45 (m, 2H), 7.41 (s, 1H), 5.87 (s, 2H), 3.98 (s, 3H), 3.94 (s, 3H), 2.33 (s, 3H), 2.24 (s, 3H), 2.13 (s, 3H), 1.46 (s, 9H); LC-MS: m/z 641.3 (M+H)⁺.

### Step-ii: N-(6-amino-5-methylpyridin-3-yl)-6-(3,5-dimethylisoxazol-4-yl)-7-methoxy-1-((3-methoxypyridin-2-yl)methyl)-2-oxo-1,2-dihydroquinoline-3-carboxamide

The process of this step was adopted from step-iii of Example-XXVIII with appropriate changes. The desired compound was obtained as yellow solid (0.03 g, 22%). ¹H NMR (400 MHz, DMSO-d₆) δ 10.87 (s, 1H), 8.92 (s, 1H), 8.19 (d, J=2.4 Hz, 1H), 8.16-8.15 (m, 1H), 8.02 (s, 1H), 7.66 (d, J=1.9 Hz, 1H), 7.58 (d, J=7.8 Hz, 1H), 7.47-7.44 (m, 1H), 7.41 (s, 1H), 5.85 (s, 2H), 5.64 (s, 2H), 3.97 (s, 3H), 3.94 (s, 3H), 2.32 (s, 3H), 2.13 (s, 3H), 2.09 (s, 3H); LC-MS: m/z 541.3 (M+H)⁺.

The below compounds were prepared by procedure similar to the one described in Example-XXX with appropriate variations in reactants, quantities of reagents and reaction conditions. The physiochemical characteristics of the compounds are also summarized.

| | | Characterization Data |
|---|---|---|
| No. | Structure | ¹H NMR (400 MHz, DMSO-d₆)/LC-MS: |
| 113 | 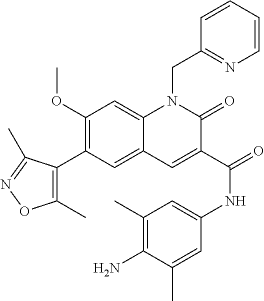 [SMILES: COc1cc2c(cc1-c1c(C)noc1C)cc(C(=O)Nc1cc(C)c(N)c(C)c1)c(=O)n2Cc1ccccn1] | ¹H NMR (400 MHz, DMSO-d₆) δ 11.61 (s, 1H), 8.94 (s, 1H), 8.50 (d, J = 4.4 Hz, 1H), 7.98 (s, 1H), 7.79 (t, J = 7.4 Hz, 1H), 7.44 (d, J = 7.8 Hz, 1H), 7.31-7.29 (m, 1H), 7.22-7.20 (m, 3H), 5.80 (s, 2H), 4.47-4.46 (bs, 2H), 3.79 (s, 3H), 2.67 (s, 3H), 2.26 (s, 3H), 2.08 (s, 6H); LC-MS: m/z 524.3 (M + H)⁺. |

### Example-XXXI: N-(1,5-dimethyl-6-oxo-1,6-dihydropyridin-3-yl)-6-(3,5-dimethylisoxazol-4-yl)-7-methoxy-2-oxo-1-(pyridin-2-ylmethyl)-1,2-dihydroquinoline-3-carboxamide (Compound-114)

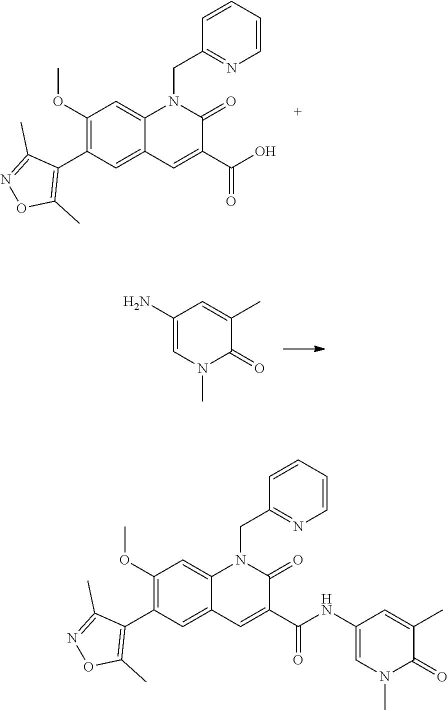

To a solution of 6-(3,5-dimethylisoxazol-4-yl)-7-methoxy-2-oxo-1-(pyridin-2-yl-methyl)-1,2-dihydroquinoline-3-carboxylic acid (0.15 g, 0.37 mmol, Intermediate-1) in DCM (5 mL) were added 5-amino-1,3-dimethylpyridin-2(1H)-one (0.1 g, 0.74 mmol, Intermediate-25), HATU (0.42 g, 1.11 mmol) and pyridine (0.09 mL, 1.11 mmol) followed by stirring at RT for 16 h. The mixture was diluted with DCM (50 mL), washed with water (50 mL) and brine (50 mL), dried over sodium sulphate and concentrated under reduced pressure. The residue was purified by combi-flash to afford the title compound as yellow solid (0.01 g). ¹H NMR (400 MHz, DMSO-d₆) δ 11.55 (s, 1H), 8.96 (s, 1H), 8.51 (d, J=4.4 Hz, 1H), 8.24 (s, 1H), 8.02 (s, 1H), 7.81 (t, J=7.9 Hz, 1H), 7.53 (s, 1H), 7.45 (d, J=7.9 Hz, 1H), 7.33-7.30 (m, 1H), 7.23 (s, 1H), 5.82 (s, 2H), 3.82 (s, 3H), 3.46 (s, 3H), 2.28 (s, 3H), 2.09 (s, 3H), 2.04 (s, 3H); LC-MS: m/z 526.2 (M+H)⁺.

The below compounds were prepared by procedure similar to the one described in Example-XXXI with appropriate variations in reactants, quantities of reagents and reaction conditions. The physiochemical characteristics of the compounds are also summarized.

| | | Characterization Data |
|---|---|---|
| No. | Structure | ¹H NMR (400 MHz, DMSO-d₆)/LC-MS: |
| 115 | 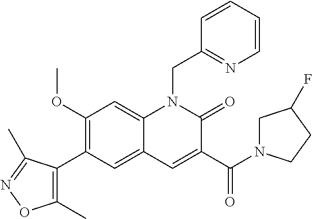 [SMILES: COc1cc2c(cc1-c1c(C)noc1C)cc(C(=O)N1CCC(F)C1)c(=O)n2Cc1ccccn1] | ¹H NMR (400 MHz, DMSO-d₆) δ 8.52 (d, J = 4.4 Hz, 1H), 8.09 (d, J = 6.4 Hz, 1H), 7.82-7.78 (m, 1H), 7.72 (s, 1H), 7.37 (d, J = 7.9 Hz, 1H), 7.31 (dd, J = 7.1 Hz & 5.1 Hz, 1H), 7.16 (d, J = 4.9 Hz, 1H), 5.71 (s, 2H), 5.42-5.20 (m, 1H), 3.78 (s, 3H), 3.76- 3.50 (m, 4H), 2.26 (s, 3H), 2.17-2.09 (m, 2H), 2.07 (s, 3H); LC-MS: m/z 477.2 (M + H)⁺. |

### Example-XXXII: 6-(3,5-Dimethylisoxazol-4-yl)-7-methoxy-1-(pyridin-2-yl-methyl)-3-((tetrahydro-2H-pyran-4-yl)oxy)quinolin-2(1H)-one (Compound-116) & 3-cyclopropoxy-6-(3,5-dimethylisoxazol-4-yl)-7-methoxy-1-(pyridin-2-ylmethyl)quinolin-2(1H)-one (Compound-117)

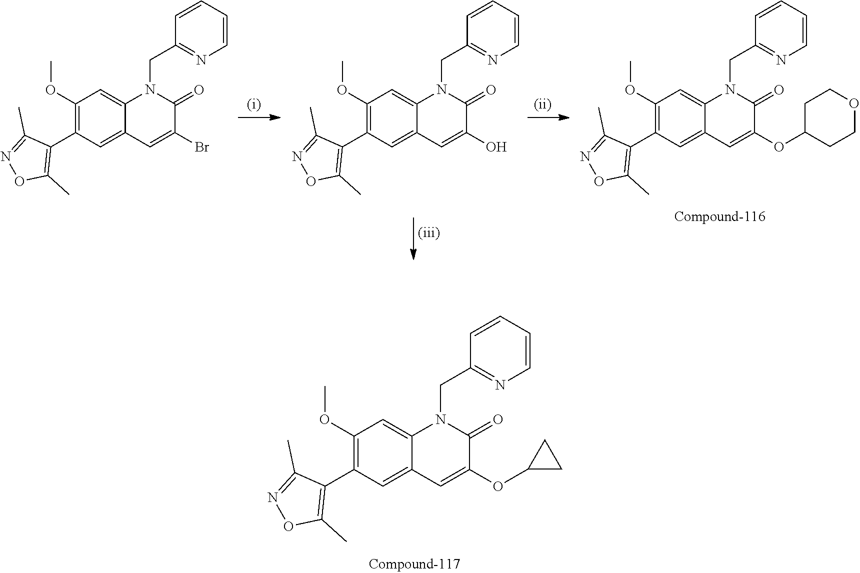 [SMILES: COc1cc2c(cc1-c1c(C)noc1C)cc(Br)c(=O)n2Cc1ccccn1 ; COc1cc2c(cc1-c1c(C)noc1C)cc(O)c(=O)n2Cc1ccccn1 ; COc1cc2c(cc1-c1c(C)noc1C)cc(OC1CCOCC1)c(=O)n2Cc1ccccn1 ; COc1cc2c(cc1-c1c(C)noc1C)cc(OC1CC1)c(=O)n2Cc1ccccn1]

### Step-i: 6-(3,5-Dimethylisoxazol-4-yl)-3-hydroxy-7-methoxy-1-(pyridin-2-yl-methyl)quinolin-2(1H)-one

To a solution of 3-bromo-6-(3,5-dimethylisoxazol-4-yl)-7-methoxy-1-(pyridin-2-ylmethyl)quinolin-2(1H)-one (0.7 g, 1.59 mmol) in 1,4-dioxane (15 mL) and H₂O (3 mL) was added KOH (0.27 g, 4.77 mmol) followed by degassing with nitrogen purging for 20 min. Then Pd₂(dba)₃ (0.15 g, 0.16 mmol) and ^{t}BuXPhos (0.07 g, 0.16 mmol) were added followed by degassing with nitrogen purging for 20 min and heating at 100° C. for 16 h. The mixture was diluted with EtOAc (100 mL), washed with water (100 mL) and brine (100 mL), dried over sodium sulphate, concentrated under reduced pressure and purified by combi-flash to afford the title compound as yellow solid (0.25 g, 42%). ¹H NMR (400 MHz, DMSO-d₆) δ 9.35 (s, 1H), 8.53 (d, J=4.4 Hz, 1H), 7.80-7.76 (m, 1H), 7.45 (s, 1H), 7.34-7.29 (m, 2H), 7.16 (s, 1H), 7.07 (s, 1H), 5.71 (s, 2H), 3.69 (s, 3H), 2.24 (s, 3H), 2.05 (s, 3H); LC-MS: m/z 378.2 (M+H)⁺.

### Step-ii: 6-(3,5-Dimethylisoxazol-4-yl)-7-methoxy-1-(pyridin-2-ylmethyl)-3-((tetrahydro-2H-pyran-4-yl)oxy)quinolin-2(1H)-one (compound 116)

To an ice cold solution of tetrahydro-2H-pyran-4-ol (0.19 g, 1.6 mmol) in THF (3 mL) was added triphenyl phosphine (0.42 g, 1.6 mmol) and DIAD (0.31 mL, 1.6 mmol) followed by stirring for 10 min. 6-(3,5-Dimethylisoxazol-4-yl)-3-hydroxy-7-methoxy-1-(pyridin-2-ylmethyl)quinolin-2(1H)-one (0.15 g, 0.4 mmol) was added followed by stirring at RT for 16 h. The mixture was diluted with EtOAc and washed with water. The organic layer was dried over sodium sulphate and concentrated under reduced pressure. The residue was purified by combi-flash to afford the title compound as an off white solid (0.02 g). ¹H NMR (400 MHz, CDCl₃) δ 8.59 (d, J=4.4 Hz, 1H), 7.64 (t, J=7.8 Hz, 1H), 7.40 (d, J=7.8 Hz, 1H), 7.27 (s, 1H), 7.24-7.20 (m, 1H), 7.18 (s, 1H), 7.03 (s, 1H), 5.73 (s, 2H), 4.64-4.58 (m, 1H), 4.12-4.02 (m, 2H), 3.79 (s, 3H), 3.63-3.57 (m, 2H), 2.27 (s, 3H), 2.13 (s, 3H), 2.12-2.10 (m, 2H), 2.05-1.91 (m, 2H); LC-MS: m/z 462.2 (M+H)⁺.

### Step-iii: 3-Cyclopropoxy-6-(3,5-dimethylisoxazol-4-yl)-7-methoxy-1-(pyridine-2-ylmethyl)quinolin-2(1H)-one (Compound 117)

In a sealed tube, to a solution of 6-(3,5-dimethylisoxazol-4-yl)-3-hydroxy-7-methoxy-1-(pyridin-2-ylmethyl)quinolin-2(1H)-one (0.1 g, 0.265 mmol) in DMF (3 mL) were added cesium carbonate (0.26 g, 0.8 mmol), KI (0.005 g, 0.026 mmol) and bromo cyclopropane (0.21 mL, 2.65 mmol) followed by heating to 170° C. for 16 h. The mixture was diluted with water and extracted with EtOAc. The organic layer was dried over sodium sulphate and concentrated under reduced pressure. The residue was purified by combi-flash to afford the title compound as pale green solid (0.02 g, 18%). ¹H NMR (400 MHz, DMSO-d₆): δ 8.52 (d, J=4.4 Hz, 1H), 7.78-7.76 (m, 1H), 7.58-7.56 (bs, 2H), 7.33-7.30 (m, 2H), 7.08 (s, 1H), 5.67 (s, 2H), 3.89-3.86 (m, 1H), 3.70 (s, 3H), 2.25 (s, 3H), 2.06 (s, 3H), 0.86-0.85 (m, 2H), 0.84-0.83 (m, 2H); LC-MS: m/z 418.2 (M+H)⁺.

Biological Data

In-Vitro Biochemical Data of bicyclic heterocyclic derivatives in time-resolved fluorescence resonance energy transfer (TR-FRET) assay.

The Bet bromodomain TR-FRET assay has been used to identify compounds that bind to Bet BRD4 bromodomain and prevent its interaction with acetylated histone peptides (Chung, C. et al., J. Med. Chem., 54, 3827-3838, 2011).

In the assay, optimized concentration of in-house Bet BRD4 bromodomain protein and 300 nM of acetyl histone peptide substrate were diluted in assay buffer (50 mM HEPES, pH: 7.5, 50 mM NaCl, 500 μM CHAPS) and were added to the positive control and test control wells in a 384 well plate. Substrate control wells have 300 nM of acetyl histone peptide substrate diluted in assay buffer. Buffer blank wells were added with assay buffer. The reaction mixture was allowed for incubation at RT for 30 min. Stock solutions of test compounds at 20 mM DMSO were prepared. Compounds were serially diluted and added to the test wells in 384-well polypropylene plates. The reaction mixture was further incubated for 30 min at RT on a plate shaker. 2 nM of Europium labeled streptavidin and 10 nM of XL-665 labeled antibody diluted in detection buffer (50 mM HEPES, pH: 7.5, 50 mM NaCl, 500 μM CHAPS and 800 mM KF) were added to all the wells excluding the buffer blank wells. The reaction plate was incubated for additional 30 min at RT on plate shaker. The plate was read in Perkin Elmer WALLAC 1420 Multilabel Counter Victor 3 (Ex: 340 nm Em: 615 and 665 nm). The amount of displacement of the peptide was measured as ratio of specific 665 nm energy transfer signal to 615 nm signals. The IC₅₀ of the compounds was determined by fitting the dose response data to sigmoid curve fitting equation using Graph Pad Prism software V5.

The compounds were screened in the above mentioned assay and the results (IC₅₀) are summarized in the table below. The IC₅₀ values of the compounds are set forth in below Table wherein “A” refers to an IC₅₀ value of less than 600 nM, “B” refers to ICso₀ value in range of 600.01 to 1000 nM and “C” refers to IC₅₀ value of in the range of 1000.01 to 10000 nM.

| Group | Compound No. |
|---|---|
| A | 1, 2, 4, 6, 7, 8, 9, 10, 12, 17, 18, 19, 20, 21, 22, 23, 24, 26, 27, |
| | 28, 29, 30, 31, 36, 37, 38, 39, 40, 41, 42, 43, 46, 48, 49, 50, 51, |
| | 52, 55, 56, 57, 58, 59, 60, 63, 64, 66, 67, 70, 71, 72, 73, 75, 77, |
| | 78, 79, 80, 81, 83, 84, 87, 88, 90, 93, 96, 99, 100, 102, 105, 106, |
| | 108, 110, 113, 114, 115, 116, 117. |
| B | 5, 11, 16, 25, 47, 61, 82, 91, 92, 94. |
| C | 3, 13, 14, 15, 32, 33, 34, 35, 44, 45, 53, 54, 62, 68, 69, 74, 76, |
| | 85, 86, 89, 95, 97, 98, 101, 103, 104, 107, 109, 111, 112. |

1. A compound of formula (I) 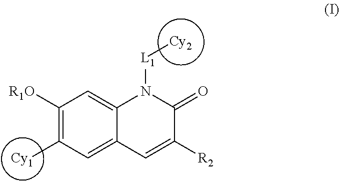
wherein,
Cy₁ is an optionally substituted 5-6 membered monocyclic heterocyclyl ring containing 1-3 hetero atoms independently selected from N or O, which ring is optionally substituted by 1-3 C₁₋₇ alkyl groups;
Cy₂ is an optionally substituted aryl, optionally substituted C₃₋₁₀ cycloalkyl or optionally substituted 5-12 membered monocyclic or bicyclic heterocyclyl ring containing 1-3 hetero atoms independently selected from N, O, or S; wherein the optional substitution at each occurrence is, independently, selected from 1-3 substituents selected from C₁₋₇ alkyl, C₁₋₇ alkoxy, halogen, and —C(O)C₁₋₇ alkyl;
L₁ is —(CR₃R₃ₐ)ₙ;
R₁ is C₁₋₇ alkyl or halo C₁₋₇ alkyl;
R₂ is an optionally substituted aryl, optionally substituted aryl C₁₋₇ alkyl, optionally substituted heterocyclyl, optionally substituted heterocyclyl C₁₋₇ alkyl, —N(Rₐ)R_{b}, —(CH₂)ₘC(O)Rₐ₁, —(CH₂)ₘ₁C(O)ORₐ₂, —(CH₂)ₘ₂C(O)N(Rₐ₃)R_{b1}, —CH(CF₃)R_{d}, —S(O)₂N(Rₐ₄)R_{b2}, —(CRₐ₅R_{b3})ₘ₃C(O)ORₐ₆, —CH(CF₃)OR_{c}, —CH(CF₃)N(Rₐ₇)R_{b4}, or —ORₑ, wherein the optional substitution at each occurrence is, independently, selected from 1-3 substituents selected from C₁₋₇ alkyl, halo C₁₋₇ alkyl, —NHC(O)C₁₋₇ alkyl, amino, halogen, hydroxy, oxo, hydroxy C₁₋₇ alkyl, aryl, —N(H)C(O)C₁₋₇ alkyl, —(CH₂)ₘ₄C(O)OH, or —(CH₂)ₘ₅C(O)NH(hydroxy C₁₋₇ alkyl);
Rₐ, Rₐ₁, Rₐ₂, Rₐ₃, Rₐ₄, Rₐ₅, Rₐ₆, Rₐ₇, R_{b}, R_{b1}, R_{b2}, R_{b3}, and R_{b4} are independently selected from hydrogen, C₁₋₇ alkyl, hydroxy, C₁₋₇ alkoxy, hydroxy C₁₋₇ alkyl, halo C₁₋₇ alkyl, —S(O)₂C₁₋₇ alkyl, optionally substituted aryl, optionally substituted C₃₋₁₀ cycloalkyl, optionally substituted heterocyclyl, or optionally substituted heterocyclyl C₁₋₇ alkyl; wherein the optional substitution at each occurrence is independently selected from 1-3 substituents selected from C₁₋₇ alkyl, halogen, hydroxy, hydroxy C₁₋₇ alkyl, C₁₋₇ alkoxy, cyano, halo C₁₋₇ alkyl, and amino;
R_{c} is selected from C₁₋₇ alkyl or aryl wherein aryl is optionally substituted by 1-3 halogen atoms;
R_{d} is selected from optionally substituted heterocyclyl or optionally substituted aryl, wherein the optional substitution at each occurrence is independently selected from 1-3 substituents selected from C₁₋₇ alkyl and halogen;
Rₑ is selected from optionally substituted C₃₋₇ cycloalkyl or optionally substituted heterocyclyl, wherein the optional substitution at each occurrence is independently selected from 1-3 substituents selected from C₁₋₇ alkyl and halogen;
R₃ and R₃ₐ independently are selected from hydrogen, C₁₋₇ alkyl, hydroxy and halogen, or alternatively R₃ and R₃ₐ together with the carbon atom to which they are attached form a carbonyl (C═O) group;
m, m₁, m₂, m₃, m₄, and m₅ are, independently, an integer selected from 0, 1, or 2; and
n is an integer selected from 1, 2, or 3;
or a pharmaceutically acceptable salt thereof.

2. The compound according to claim 1, wherein Cy₁ is 3,5-dimethylisoxazole.

3. The compound according to claim 1, wherein R₁ is C₁₋₇ alkyl.

4. The compound according to claim 3, wherein R₁ is methyl.

5. The compound according to claim 1, wherein Cy₂ is a 5-12 membered monocyclic or bicyclic ring containing 0-2 hetero atoms independently selected from N and O, which ring is optionally substituted by 1-3 substituents selected from C₁₋₇ alkyl, C₁₋₇ alkoxy, halogen, and —C(O)C₁₋₇ alkyl.

6. The compound according to claim 5, wherein Cy₂ is selected from optionally substituted pyridyl, optionally substituted phenyl, cyclohexyl, morpholinyl, optionally substituted piperazinyl, or optionally substituted chromanyl; wherein the optional substitution at each occurrence is independently selected from 1-3 substituents selected from C₁₋₇ alkyl, C₁₋₇ alkoxy, halogen, and —C(O)C₁₋₇ alkyl.

7. The compound according to claim 5, wherein Cy₂ is optionally substituted by 1-2 substituents selected from C₁₋₇ alkoxy and halogen.

8. The compound according to claim 6, wherein Cy₂ is selected from optionally substituted pyridyl or optionally substituted phenyl, wherein the optional substitution at each occurrence is independently selected from 1-2 substituents selected from C₁₋₇ alkoxy and halogen.

9. The compound according to claim 1, wherein L₁ is —CH₂—, —(CH₂)₂—, —CH₂CH(OH)—, —CH₂CH(CH₃)— or —CH₂C(O)—, wherein the left bond is attached to the quinolin-2(1H)-one ring of formula (I).

10. The compound according to claim 1, wherein R₂ is an optionally substituted 5-12 membered monocyclic or bicyclic ring containing 0-4 hetero atoms independently selected from N and O, which ring is optionally substituted by 1-3 substituents selected from C₁₋₇ alkyl, halogen, amino, hydroxy, —NHC(O)C₁₋₇ alkyl, halo C₁₋₇ alkyl, phenyl, oxo, hydroxy C₁₋₇ alkyl, —(CH₂)ₘ₅C(O)NH(hydroxy C₁₋₇ alkyl), or —(CH₂)ₘ₄C(O)OH.

11. The compound according to claim 10, wherein R₂ is phenyl, isoxazolyl, pyridinyl, pyrazolyl, imidazolyl, morpholinyl, 3,4-dihydroisoquinolinyl, 1,2,3,4-tetrahydroisoquinolinyl, 2-oxoimidazolidinyl, piperidinyl, pyrrolidinyl, indolinyl, 1,2,4-oxadiazol-5-yl or 1H-benzo[d]imidazole, or azetidinyl; and the optional substituents are selected from 1-3 substituents selected from C₁₋₇ alkyl, halogen, amino, hydroxy, NHC(O)C₁₋₇ alkyl, halo C₁₋₇ alkyl, phenyl, oxo, hydroxy C₁₋₇ alkyl, —(CH₂)ₘ₅C(O)NH(hydroxy C₁₋₇ alkyl), or —(CH₂)ₘ₄C(O)OH.

12. The compound according to claim 1, wherein R₂ is —(CH₂)ₘC(O)Rₐ₁.

13. The compound according to claim 12, wherein Rₐ₁ is a 5-12 membered monocyclic or bicyclic ring containing 0-4 hetero atoms independently selected from N and O, which ring is optionally substituted by one hydroxy or halogen group, and m is 0 or 1.

14. The compound according to claim 13, wherein Rₐ₁ is phenyl, piperidinyl, pyrrolidinyl, azetidinyl or indolinyl which rings are optionally substituted by one hydroxy or halogen group, and m is 0 or 1.

15. The compound according to claim 1, wherein R₂ is —(CH₂)ₘ₂C(O)N(Rₐ₃)R_{b1}.

16. The compound according to claim 15, wherein Rₐ₃ is hydrogen or C₁₋₇ alkyl, and R_{b1} is hydrogen, C₁₋₇ alkyl, hydroxy C₁₋₇ alkyl, halo C₁₋₇ alkyl, optionally substituted C₃₋₁₀ cycloalkyl, optionally substituted heterocyclyl, optionally substituted phenyl, or optionally substituted heterocyclyl C₁₋₇ alkyl, wherein heterocyclyl at each occurrence means a 5-12 membered monocyclic or bicyclic ring containing 1-4 hetero atoms independently selected from N, O, and S, and wherein the optional substitution at each occurrence is, independently, selected from 1-3 substituents selected from C₁₋₇ alkyl, hydroxy, halogen, halo C₁₋₇ alkyl, amino, cyano, C₁₋₇ alkoxy, or oxo; and m2 is 0 or 1.

17. The compound according to claim 15, wherein R_{b1} is cyclohexyl, pyridinyl, piperidinyl, 1,3,4-thiadiazolyl, pyrazolyl, phenyl, or imidazolyl C₁₋₇ alkyl, which groups are optionally substituted by 1-3 substituents independently selected from C₁₋₇ alkyl, hydroxy, halogen, halo C₁₋₇ alkyl, amino, cyano, C₁₋₇ alkoxy, or oxo.

18. The compound according to claim 15, wherein Rₐ₃ is hydrogen.

19. The compound according to claim 1, wherein R₂ is —N(Rₐ)R_{b}.

20. The compound according to claim 19, wherein Rₐ is hydrogen and R_{b} is hydrogen, hydroxy C₁₋₇ alkyl, —SO₂-methyl, or optionally substituted 5-12 membered monocyclic or bicyclic ring containing 1-4 hetero atoms independently selected from N, O, and S, and wherein the optional substitution is selected from 1-3 substituents selected from C₁₋₇ alkyl, hydroxy, halogen, halo C₁₋₇ alkyl, or C₁₋₇ alkoxy.

21. The compound according to claim 1, wherein R₂ is —CH(CF₃)R_{d}, —CH(CF₃)OR_{c}, or —CH(CF₃)N(Rₐ₇)R_{b4}.

22. The compound according to claim 21, wherein R_{d} is morpholinyl, R_{c} of is 4-fluorophenyl or C₁₋₇ alkyl, Rₐ₇ is hydrogen, and R_{b4} is hydroxy C₁₋₇ alkyl or 4-fluorophenyl.

23. The compound according to claim 1, wherein n is 1 or 2.

24. The compound according to claim 1, wherein the compound of formula (I) is a compound of formula (IA): 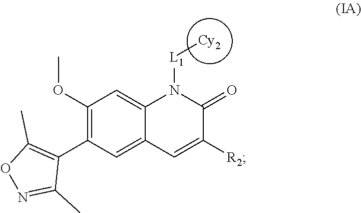
or a pharmaceutically acceptable salt thereof.

25. The compound according to claim 1, wherein the compound of formula (I) is a compound of formula (IB): 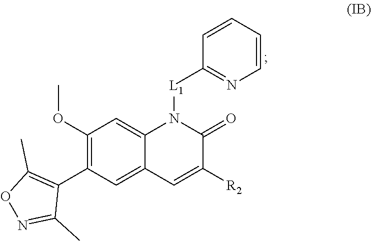
or a pharmaceutically acceptable salt thereof.

26. The compound according to claim 1, wherein the compound of formula (I) is a compound of formula (IC): 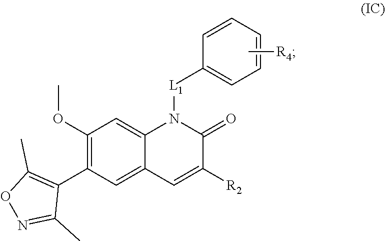
R₄ is hydrogen, C₁₋₇ alkoxy, or halogen, or a pharmaceutically acceptable salt thereof.

27. The compound according to claim 1, wherein L₁ is —CH₂—.

28. The compound according to claim 1, wherein heterocyclyl group, at each occurrence, independently, is a 5-12 membered monocyclic or bicyclic ring containing 1-4 hetero atoms independently selected from N, O, and S.

29. The compound according to claim 1 selected from the group consisting of

| | |
|---|---|
| 1. | 3,6-Bis(3,5-dimethylisoxazol-4-yl)-7-methoxy-1-(pyridin-2- |
| | ylmethyl)-quinolin-2(1H)-one; |
| 2. | 6-(3,5-Dimethylisoxazol-4-yl)-7-methoxy-1-(pyridin-2-ylmethyl)- |
| | 3-(pyridin-4-yl)quinolin-2(1H)-one; |
| 3. | 6-(3,5-Dimethylisoxazol-4-yl)-7-methoxy-3-(naphthalen-2-yl)-1- |
| | (pyridin-2-ylmethyl)quinolin-2(1H)-one; |
| 4. | N-(3-(6-(3,5-dimethylisoxazol-4-yl)-7-methoxy-2-oxo-1-(pyridin- |
| | 2-yl-methyl)-1,2-dihydroquinolin-3-yl)phenyl)acetamide; |
| 5. | 6-(3,5-Dimethylisoxazol-4-yl)-7-methoxy-3-phenyl-1-(pyridin-2- |
| | ylmethyl)-quinolin-2(1H)-one; |
| 6. | 3-(2,4-Difluorophenyl)-6-(3,5-dimethylisoxazol-4-yl)-7-methoxy- |
| | 1-(pyridin-2-ylmethyl)quinolin-2(1H)-one; |
| 7. | 6-(3,5-Dimethylisoxazol-4-yl)-3-(4-hydroxyphenyl)-7-methoxy- |
| | 1-(pyridin-2-ylmethyl)quinolin-2(1H)-one; |
| 8. | 6-(3,5-Dimethylisoxazol-4-yl)-7-methoxy-1-(pyridin-2-ylmethyl)- |
| | 3-(2-(tri-fluoromethyl)phenyl)quinolin-2(1H)-one; |
| 9. | 6-(3,5-Dimethylisoxazol-4-yl)-7-methoxy-3-(1-methyl-1H- |
| | pyrazol-4-yl)-1-(pyridin-2-ylmethyl)quinolin-2(1H)-one; |
| 10. | 6-(3,5-Dimethylisoxazol-4-yl)-7-methoxy-1-(pyridin-2-ylmethyl)- |
| | 3-(pyridin-3-yl)quinolin-2(1H)-one; |
| 11. | 6-(3,5-Dimethylisoxazol-4-yl)-3-(5-(hydroxymethyl)-3-methyl- |
| | isoxazol-4-yl)-7-methoxy-1-(pyridin-2-ylmethyl)quinolin-2(1H)- |
| | one; |
| 12. | 6-(3,5-Dimethylisoxazol-4-yl)-7-methoxy-3-(1H-pyrazol-4-yl)-1- |
| | (pyridin-2-ylmethyl)quinolin-2(1H)-one; |
| 13. | 1-(4-Chlorophenethyl)-6-(3,5-dimethylisoxazol-4-yl)-3-(4- |
| | hydroxyphenyl)-7-methoxyquinolin-2(1H)-one; |
| 14. | 1-(4-Chlorophenethyl)-6-(3,5-dimethylisoxazol-4-yl)-7-methoxy- |
| | 3-(1-methyl-1H-pyrazol-4-yl)quinolin-2(1H)-one; |
| 15. | 6-(3,5-Dimethylisoxazol-4-yl)-3-(1H-imidazol-1-yl)-7-methoxy- |
| | 1-(pyridin-2-ylmethyl)quinolin-2(1H)-one; |
| 16. | 6-(3,5-Dimethylisoxazol-4-yl)-7-methoxy-3-(1H-pyrazol-1-yl)-1- |
| | (pyridin-2-ylmethyl)quinolin-2(1H)-one; |
| 17. | 6-(3,5-Dimethylisoxazol-4-yl)-7-methoxy-3-(4-phenyl-1H- |
| | imidazol-1-yl)-1-(pyridin-2-ylmethyl)quinolin-2(1H)-one; |
| 18. | 6-(3,5-Dimethylisoxazol-4-yl)-7-methoxy-3-morpholino-1- |
| | (pyridin-2-yl-methyl)quinolin-2(1H)-one; |
| 19. | 6-(3,5-Dimethylisoxazol-4-yl)-3-((2-hydroxyethyl)amino)-7- |
| | methoxy-1-(pyridin-2-ylmethyl)quinolin-2(1H)-one; |
| 20. | N-(6-(3,5-dimethylisoxazol-4-yl)-7-methoxy-2-oxo-1-(pyridin-2- |
| | ylmethyl)-1,2-dihydroquinolin-3-yl)methanesulfonamide; |
| 21. | 3-(3,4-Dihydroisoquinolin-2(1H)-yl)-6-(3,5-dimethylisoxazol-4- |
| | yl)-7-methoxy-1-(pyridin-2-ylmethyl)quinolin-2(1H)-one; |
| 22. | 6-(3,5-Dimethylisoxazol-4-yl)-7-methoxy-3-((1-methylpiperidin- |
| | 4-yl)-amino)-1-(pyridin-2-ylmethyl)quinolin-2(1H)-one; |
| 23. | 6-(3,5-Dimethylisoxazol-4-yl)-7-methoxy-3-(2-oxoimidazolidin- |
| | 1-yl)-1-(pyridin-2-ylmethyl)quinolin-2(1H)-one; |
| 24. | 1-(4-Chlorophenethyl)-6-(3,5-dimethylisoxazol-4-yl)-7-methoxy- |
| | 3-(2-oxo-imidazolidin-1-yl)quinolin-2(1H)-one; |
| 25. | 6-(3,5-Dimethylisoxazol-4-yl)-3-(4-hydroxypiperidine-1- |
| | carbonyl)-7-methoxy-1-(pyridin-2-ylmethyl)quinolin-2(1H)-one; |
| 26. | 6-(3,5-Dimethylisoxazol-4-yl)-3-(3-hydroxypyrrolidine-1- |
| | carbonyl)-7-methoxy-1-(pyridin-2-ylmethyl)quinolin-2(1H)-one; |
| 27. | 6-(3,5-Dimethylisoxazol-4-yl)-N-((1r,4r)-4-hydroxycyclohexyl)- |
| | 7-methoxy-2-oxo-1-(pyridin-2-ylmethyl)-1,2-dihydroquinoline-3- |
| | carboxamide; |
| 28. | 6-(3,5-Dimethylisoxazol-4-yl)-N-(5-(hydroxymethyl)-1,3,4- |
| | thiadiazol-2-yl)-7-methoxy-2-oxo-1-(pyridin-2-ylmethyl)-1,2- |
| | dihydroquinoline-3-carboxamide; |
| 29. | 6-(3,5-Dimethylisoxazol-4-yl)-N-ethyl-7-methoxy-2-oxo-1- |
| | (pyridin-2-yl-methyl)-1,2-dihydroquinoline-3-carboxamide; |
| 30. | 6-(3,5-Dimethylisoxazol-4-yl)-N-(3-hydroxypropyl)-7-methoxy- |
| | 2-oxo-1-(pyridin-2-ylmethyl)-1,2-dihydroquinoline-3- |
| | carboxamide; |
| 31. | 6-(3,5-Dimethylisoxazol-4-yl)-7-methoxy-N-methyl-2-oxo-1- |
| | (pyridin-2-yl-methyl)-1,2-dihydroquinoline-3-carboxamide; |
| 32. | 6-(3,5-Dimethylisoxazol-4-yl)-7-methoxy-2-oxo-1-(pyridin-2- |
| | ylmethyl)-N-(5-(trifluoromethyl)-1,3,4-thiadiazol-2-yl)-1,2- |
| | dihydroquinoline-3-carboxamide; |
| 33. | 6-(3,5-Dimethylisoxazol-4-yl)-N-(4-fluorophenyl)-7-methoxy-2- |
| | oxo-1-(pyridin-2-ylmethyl)-1,2-dihydroquinoline-3-carboxamide; |
| 34. | 6-(3,5-Dimethylisoxazol-4-yl)-7-methoxy-2-oxo-1-(pyridin-2- |
| | ylmethyl)-N-(4-(trifluoromethyl)phenyl)-1,2-dihydroquinoline-3- |
| | carboxamide; |
| 35. | 6-(3,5-Dimethylisoxazol-4-yl)-7-methoxy-N-(4-methoxyphenyl)- |
| | 2-oxo-1-(pyridin-2-ylmethyl)-1,2-dihydroquinoline-3- |
| | carboxamide; |
| 36. | 6-(3,5-Dimethylisoxazol-4-yl)-7-methoxy-2-oxo-1-(pyridin-2- |
| | ylmethyl)-N-(2,2,2-trifluoroethyl)-1,2-dihydroquinoline-3- |
| | carboxamide; |
| 37. | 3-(Azetidine-1-carbonyl)-6-(3,5-dimethylisoxazol-4-yl)-7- |
| | methoxy-1-(pyridin-2-ylmethyl)quinolin-2(1H)-one; |
| 38. | 6-(3,5-Dimethylisoxazol-4-yl)-7-methoxy-2-oxo-1-(pyridin-2- |
| | ylmethyl)-N-(pyridin-4-yl)-1,2-dihydroquinoline-3-carboxamide; |
| 39. | N-(3-(1H-imidazol-1-yl)propyl)-6-(3,5-dimethylisoxazol-4-yl)-7- |
| | methoxy-2-oxo-1-(pyridin-2-ylmethyl)-1,2-dihydroquinoline-3- |
| | carboxamide; |
| 40. | 6-(3,5-Dimethylisoxazol-4-yl)-N-(4-hydroxyphenyl)-7-methoxy- |
| | 2-oxo-1-(pyridin-2-ylmethyl)-1,2-dihydroquinoline-3- |
| | carboxamide; |
| 41. | 6-(3,5-Dimethylisoxazol-4-yl)-7-methoxy-2-oxo-N-(pyridin-2-yl)- |
| | 1-(pyridin-2-ylmethyl)-1,2-dihydroquinoline-3-carboxamide; |
| 42. | 6-(3,5-Dimethylisoxazol-4-yl)-7-methoxy-2-oxo-1-(pyridin-2- |
| | ylmethyl)-N-(pyridin-3-yl)-1,2-dihydroquinoline-3-carboxamide; |
| 43. | 6-(3,5-Dimethylisoxazol-4-yl)-7-methoxy-N-(1-methyl-1H- |
| | pyrazol-3-yl)-2-oxo-1-(pyridin-2-ylmethyl)-1,2-dihydroquinoline- |
| | 3-carboxamide; |
| 44. | N-(4-Chlorophenyl)-6-(3,5-dimethylisoxazol-4-yl)-7-methoxy-2- |
| | oxo-1-(pyridin-2-ylmethyl)-1,2-dihydroquinoline-3-carboxamide; |
| 45. | N-(4-Cyanophenyl)-6-(3,5-dimethylisoxazol-4-yl)-7-methoxy-2- |
| | oxo-1-(pyridin-2-ylmethyl)-1,2-dihydroquinoline-3-carboxamide; |
| 46. | 6-(3,5-Dimethylisoxazol-4-yl)-3-(5-hydroxyindoline-1-carbonyl)- |
| | 7-methoxy-1-(pyridin-2-ylmethyl)quinolin-2(1H)-one; |
| 47. | 6-(3,5-Dimethylisoxazol-4-yl)-7-methoxy-N-(1-methylpiperidin- |
| | 4-yl)-2-oxo-1-(pyridin-2-ylmethyl)-1,2-dihydroquinoline-3- |
| | carboxamide; |
| 48. | 6-(3,5-Dimethylisoxazol-4-yl)-N-(4-hydroxy-3-methylphenyl)-7- |
| | methoxy-2-oxo-1-(pyridin-2-ylmethyl)-1,2-dihydroquinoline-3- |
| | carboxamide; |
| 49. | 6-(3,5-Dimethylisoxazol-4-yl)-3-(3-hydroxyazetidine-1-carbonyl)- |
| | 7-methoxy-1-(pyridin-2-ylmethyl)quinolin-2(1H)-one; |
| 50. | 6-(3,5-Dimethylisoxazol-4-yl)-N-(6-hydroxypyridin-3-yl)-7- |
| | methoxy-2-oxo-1-(pyridin-2-ylmethyl)-1,2-dihydroquinoline-3- |
| | carboxamide; |
| 51. | 6-(3,5-Dimethylisoxazol-4-yl)-N-(3-fluoro-4-hydroxyphenyl)-7- |
| | methoxy-2-oxo-1-(pyridin-2-ylmethyl)-1,2-dihydroquinoline-3- |
| | carboxamide; |
| 52. | 2-(6-(3,5-Dimethylisoxazol-4-yl)-7-methoxy-2-oxo-1-(pyridin-2- |
| | ylmethyl)-1,2-dihydroquinolin-3-yl)-N-(4-hydroxyphenyl) |
| | acetamide; |
| 53. | 6-(3,5-Dimethylisoxazol-4-yl)-N-(3-hydroxyphenyl)-7-methoxy- |
| | 2-oxo-1-(pyridin-2-ylmethyl)-1,2-dihydroquinoline-3- |
| | carboxamide; |
| 54. | 6-(3,5-Dimethylisoxazol-4-yl)-N-(4-hydroxyphenyl)-7-methoxy- |
| | N-methyl-2-oxo-1-(pyridin-2-ylmethyl)-1,2-dihydroquinoline-3- |
| | carboxamide; |
| 55. | 6-(3,5-Dimethylisoxazol-4-yl)-N-(4-hydroxy-3,5-dimethylphenyl)- |
| | 7-methoxy-2-oxo-1-(pyridin-2-ylmethyl)-1,2-dihydroquinoline-3- |
| | carboxamide; |
| 56. | 2-(6-(3,5-Dimethylisoxazol-4-yl)-7-methoxy-2-oxo-1-(pyridin-2- |
| | ylmethyl)-1,2-dihydroquinolin-3-yl)-N-ethylacetamide; |
| 57. | 3-(2-(Azetidin-1-yl)-2-oxoethyl)-6-(3,5-dimethylisoxazol-4-yl)-7- |
| | methoxy-1-(pyridin-2-ylmethyl)quinolin-2(1H)-one; |
| 58. | 2-(6-(3,5-Dimethylisoxazol-4-yl)-7-methoxy-2-oxo-1-(pyridin-2- |
| | ylmethyl)-1,2-dihydroquinolin-3-yl)acetamide; |
| 59. | 2-(6-(3,5-Dimethylisoxazol-4-yl)-7-methoxy-2-oxo-1-(pyridin-2- |
| | ylmethyl)-1,2-dihydroquinolin-3-yl)-N-(4-hydroxy-3,5- |
| | dimethylpheny)acetamide; |
| 60. | 6-(3,5-Dimethylisoxazol-4-yl)-N-(2,6-dimethylpyridin-4-yl)-7- |
| | methoxy-2-oxo-1-(pyridin-2-ylmethyl)-1,2-dihydroquinoline-3- |
| | carboxamide, |
| 61. | 6-(3,5-Dimethylisoxazol-4-yl)-N,N-diethyl-7-methoxy-2-oxo-1- |
| | (pyridin-2-ylmethyl)-1,2-dihydroquinoline-3-carboxamide; |
| 62. | 6-(3,5-Dimethylisoxazol-4-yl)-N-(4,6-dimethylpyridin-2-yl)-7- |
| | methoxy-2-oxo-1-(pyridin-2-ylmethyl)-1,2-dihydroquinoline-3- |
| | carboxamide; |
| 63. | N-(6-aminopyridin-3-yl)-6-(3,5-dimethylisoxazol-4-yl)-7-methoxy- |
| | 2-oxo-1-(pyridin-2-ylmethyl)-1,2-dihydroquinoline-3- |
| | carboxamide; |
| 64. | 1-(2,4-Difluorobenzyl)-6-(3,5-dimethylisoxazol-4-yl)-N-(2,6- |
| | dimethyl-pyridin-4-yl)-7-methoxy-2-oxo-1,2-dihydroquinoline-3- |
| | carboxamide; |
| 65. | 6-(3,5-Dimethylisoxazol-4-yl)-N-(2,6-dimethylpyridin-4-yl)-7- |
| | methoxy-1-((3-methoxypyridin-2-yl)methyl)-2-oxo-1,2- |
| | dihydroquinoline-3-carboxamide |
| 66. | 6-(3,5-Dimethylisoxazol-4-yl)-3-(1H-imidazol-2-yl)-7-methoxy-1- |
| | (pyridin-2-ylmethyl)quinolin-2(1H)-one; |
| 67. | 6-(3,5-Dimethylisoxazol-4-yl)-7-methoxy-3-(1-phenyl-1H- |
| | imidazol-2-yl)-1-(pyridin-2-ylmethyl)quinolin-2(1H)-one; |
| 68. | 6-(3,5-Dimethylisoxazol-4-yl)-3-(hydroxydiphenylmethyl)-7- |
| | methoxy-1-(pyridin-2-ylmethyl)quinolin-2(1H)-one; |
| 69. | 6-(3,5-Dimethylisoxazol-4-yl)-7-methoxy-1-(pyridin-2-ylmethyl)- |
| | 3-(2,2,2-trifluoro-1-(4-fluorophenoxy)ethyl)quinolin-2(1H)-one; |
| 70. | 6-(3,5-Dimethylisoxazol-4-yl)-7-methoxy-1-(pyridin-2-ylmethyl)- |
| | 3-(2,2,2-trifluoro-1-morpholinoethyl)quinolin-2(1H)-one; |
| 71. | 6-(3,5-Dimethylisoxazol-4-yl)-7-methoxy-1-(pyridin-2-ylmethyl)- |
| | 3-(2,2,2-trifluoro-1-((2-hydroxyethyl)amino)ethyl)quinolin-2(1H)- |
| | one; |
| 72. | 7-Methoxy-6-(3-methylisoxazol-4-yl)-1-(pyridin-2-ylmethyl)-3- |
| | (2,2,2-trifluoro-1-((4-hydroxyphenyl)amino)ethyl)quinolin-2(1H)- |
| | one; |
| 73. | 6-(3,5-Dimethylisoxazol-4-yl)-3-(1-ethoxy-2,2,2-trifluoroethyl)-7- |
| | methoxy-1-(pyridin-2-ylmethyl)quinolin-2(1H)-one; |
| 74. | 6-(3,5-Dimethylisoxazol-4-yl)-7-methoxy-1-(pyridin-2-ylmethyl)- |
| | 3-(2,2,2-trifluoro-1-isobutoxyethyl)quinolin-2(1H)-one; |
| 75. | 6-(3,5-Dimethylisoxazol-4-yl)-3-(3-ethyl-1,2,4-oxadiazol-5-yl)-7- |
| | methoxy-1-(pyridin-2-ylmethyl)quinolin-2(1H)-one; |
| 76. | 3-Benzoyl-6-(3,5-dimethylisoxazol-4-yl)-7-methoxy-1-(pyridin-2- |
| | ylmethyl)quinolin-2(1H)-one; |
| 77. | 2-(6-(3,5-Dimethylisoxazol-4-yl)-7-methoxy-2-oxo-1-(pyridin-2- |
| | ylmethyl)-1,2-dihydroquinolin-3-yl)acetic acid; |
| 78. | 2-(1-(4-Chlorophenethyl)-6-(3,5-dimethylisoxazol-4-yl)-7- |
| | methoxy-2-oxo-1,2-dihydroquinolin-3-yl)acetic acid; |
| 79. | 2-(6-(3,5-Dimethylisoxazol-4-yl)-7-methoxy-1-(4- |
| | methoxybenzyl)-2-oxo-1,2-dihydroquinolin-3-yl)acetic acid; |
| 80. | 2-(4-(6-(3,5-Dimethylisoxazol-4-yl)-7-methoxy-2-oxo-1-(pyridin- |
| | 2-yl-methyl)-1,2-dihydroquinolin-3-yl)-1H-pyrazol-1-yl)-N-(2- |
| | hydroxyethyl)-acetamide; |
| 81. | 2-(4-(6-(3,5-Dimethylisoxazol-4-yl)-7-methoxy-2-oxo-1-(pyridin- |
| | 2-yl-methyl)-1,2-dihydroquinolin-3-yl)-1H-pyrazol-1- |
| | yl)acetic acid; |
| 82. | 6-(3,5-Dimethylisoxazol-4-yl)-7-methoxy-2-oxo-N-(pyridin-2-yl)- |
| | 1-(pyridin-2-ylmethyl)-1,2-dihydroquinoline-3-sulfonamide; |
| 83. | 6-(3,5-Dimethylisoxazol-4-yl)-3-(6-hydroxy-1H-benzo[d]imidazol- |
| | 2-yl)-7-methoxy-1-(pyridin-2-ylmethyl)quinolin-2(1H)-one; |
| 84. | 3-(Azetidine-1-carbonyl)-1-(4-chlorobenzyl)-6-(3,5- |
| | dimethylisoxazol-4-yl)-7-methoxyquinolin-2(1H)-one; |
| 85. | 3-(Azetidine-1-carbonyl)-6-(3,5-dimethylisoxazol-4-yl)-7- |
| | methoxy-1-(2-morpholinoethyl)quinolin-2(1H)-one; |
| 86. | 3-(Azetidine-1-carbonyl)-6-(3,5-dimethylisoxazol-4-yl)-7- |
| | methoxy-1-(2-(4-pivaloylpiperazin-1-yl)ethyl)quinolin-2(1H)- |
| | one; |
| 87. | 3-(Azetidine-1-carbonyl)-1-benzyl-6-(3,5-dimethylisoxazol-4-yl)- |
| | 7-methoxyquinolin-2(1H)-one; |
| 88. | 3-(Azetidine-1-carbonyl)-1-(4-chlorophenethyl)-6-(3,5-dimethyl- |
| | isoxazol-4-yl)-7-methoxyquinolin-2(1H)-one; |
| 89. | 3-(Azetidine-1-carbonyl)-1-(3,4-dichlorophenethyl)-6-(3,5- |
| | dimethylisoxazol-4-yl)-7-methoxyquinolin-2(1H)-one; |
| 90. | 3-(Azetidine-1-carbonyl)-6-(3,5-dimethylisoxazol-4-yl)-1-(4- |
| | fluorophen-ethyl)-7-methoxyquinolin-2(1H)-one; |
| 91. | 3-(Azetidine-1-carbonyl)-6-(3,5-dimethylisoxazol-4-yl)-7- |
| | methoxy-1-(2-(pyridin-3-yl)ethyl)quinolin-2(1H)-one; |
| 92. | 3-(Azetidine-1-carbonyl)-1-(2-(4-chlorophenyl)propyl)-6-(3,5- |
| | dimethyl-isoxazol-4-yl)-7-methoxyquinolin-2(1H)-one; |
| 93. | 3-(Azetidine-1-carbonyl)-1-(2-(4-chlorophenyl)-2-hydroxyethyl)- |
| | 6-(3,5-di-methylisoxazol-4-yl)-7-methoxyquinolin-2(1H)-one; |
| 94. | 3-(Azetidine-1-carbonyl)-1-(2-cyclohexylethyl)-6-(3,5- |
| | dimethylisoxazol-4-yl)-7-methoxyquinolin-2(1H)-one; |
| 95. | 3-(Azetidine-1-carbonyl)-1-(2-(2,2-dimethylchroman-6-yl)ethyl)- |
| | 6-(3,5-di-methylisoxazol-4-yl)-7-methoxyquinolin-2(1H)-one; |
| 96. | 3-(Azetidine-1-carbonyl)-6-(3,5-dimethylisoxazol-4-yl)-7- |
| | methoxy-1-phenethylquinolin-2(1H)-one; |
| 97. | 3-(Azetidine-1-carbonyl)-1-(3-chlorophenethyl)-6-(3,5- |
| | dimethylisoxazol-4-yl)-7-methoxyquinolin-2(1H)-one; |
| 98. | 6-(3,5-Dimethylisoxazol-4-yl)-N-(4-hydroxy-3,5-dimethylphenyl)- |
| | 7-methoxy-N-methyl-2-oxo-1-(pyridin-2-ylmethyl)-1,2- |
| | dihydroquinoline-3-carboxamide; |
| 99. | 6-(3,5-Dimethylisoxazol-4-yl)-3-((5-hydroxyindolin-1-yl)methyl)- |
| | 7-methoxy-1-(pyridin-2-ylmethyl)quinolin-2(1H)-one; |
| 100. | 3-((1H-tetrazol-5-yl)methyl)-6-(3,5-dimethylisoxazol-4-yl)-7- |
| | methoxy-1-(pyridin-2-ylmethyl)quinolin-2(1H)-one; |
| 101. | 2-(6-(3,5-Dimethylisoxazol-4-yl)-7-methoxy-2-oxo-1-(pyridin-2- |
| | ylmethyl)-1,2-dihydroquinolin-3-yl)-2-methylpropanoic acid; |
| 102. | 2-(1-(4-Chlorophenethyl)-6-(3,5-dimethylisoxazol-4-yl)-7- |
| | methoxy-2-oxo-1,2-dihydroquinolin-3-yl)-2-methylpropanoic acid; |
| 103. | 3-(azetidine-1-carbonyl)-1-(2-(4-chlorophenyl)-2-oxoethyl)-6-(3,5- |
| | dimethylisoxazol-4-yl)-7-methoxyquinolin-2(1H)-one; |
| 104. | 6-(3,5-Dimethylisoxazol-4-yl)-N-(4,6-dimethylpyridin-2-yl)-7- |
| | methoxy-2-oxo-1-(pyridin-2-ylmethyl)-1,2-dihydroquinoline-3- |
| | carboxamide hydrochloride; |
| 105. | 3-(6-Aminopyridin-3-yl)-6-(3,5-dimethylisoxazol-4-yl)-7- |
| | methoxy-1-(pyridin-2-ylmethyl)quinolin-2(1H)-one; |
| 106. | N-(6-Amino-5-methylpyridin-3-yl)-6-(3,5-dimethylisoxazol-4-yl)- |
| | 7-methoxy-2-oxo-1-(pyridin-2-ylmethyl)-1,2-dihydroquinoline-3- |
| | carboxamide; |
| 107. | 6-(3,5-Dimethylisoxazol-4-yl)-3((2,6-dimethylpyridin-4-yl) |
| | amino)-7-methoxy-1-(pyridin-2-ylmethyl)quinolin-2(1H)-one; |
| 108. | 3-(6-Amino-5-methylpyridin-3-yl)-6-(3,5-dimethylisoxazol-4-yl)- |
| | 7-methoxy-1-(pyridin-2-ylmethyl)quinolin-2(1H)-one; |
| 109. | 3-(Azetidine-1-carbonyl)-6-(3,5-dimethylisoxazol-4-yl)-7- |
| | methoxy-1-((3-methoxypyridin-2-yl)methyl)quinolin-2(1H)-one; |
| 110. | 3-(6-Aminopyridin-3-yl)-6-(3,5-dimethylisoxazol-4-yl)-7- |
| | methoxy-1-((3-methoxypyridin-2-yl)methyl)quinolin-2(1H)-one; |
| 111. | 6-(3,5-Dimethylisoxazol-4-yl)-N-(4-hydroxy-3,5-dimethylphenyl)- |
| | 7-methoxy-1-((3-methoxypyridin-2-yl)methyl)-2-oxo-1,2- |
| | dihydroquinoline-3-carboxamide; |
| 112. | N-(6-amino-5-methylpyridin-3-yl)-6-(3,5-dimethylisoxazol-4-yl)- |
| | 7-methoxy-1-((3-methoxypyridin-2-yl)methyl)-2-oxo-1,2- |
| | dihydroquinoline-3-carboxamide |
| 113. | N-(4-amino-3,5-dimethylphenyl)-6-(3,5-dimethylisoxazol-4-yl)-7- |
| | methoxy-2-oxo-1-(pyridin-2-ylmethyl)-1,2-dihydroquinoline-3- |
| | carboxamide |
| 114. | N-(1,5-dimethyl-6-oxo-1,6-dihydropyridin-3-yl)-6-(3,5- |
| | dimethylisoxazol-4-yl)-7-methoxy-2-oxo-1-(pyridin-2-ylmethyl)- |
| | 1,2-dihydroquinoline-3-carboxamide |
| 115. | 6-(3,5-Dimethylisoxazol-4-yl)-3-(3-fluoropyrrolidine-1-carbonyl)- |
| | 7-methoxy-1-(pyridin-2-ylmethyl)quinolin-2(1H)-one |
| 116. | 6-(3,5-Dimethylisoxazol-4-yl)-7-methoxy-1-(pyriolin-2-ylmethyl)- |
| | 3-((tetrahydro-2H-pyran-4-yl)oxy)quinolin-2(1H)-one |
| 117. | 3-Cyclopropoxy-6-(3,5-dimethylisoxazol-4-yl)-7-methoxy-1- |
| | (pyridin-2-yl-methyl)quinolin-2(1H)-one |

or a pharmaceutically acceptable salt or tautomer thereof.

30. A pharmaceutical composition comprising a compound according to claim 1 together with a pharmaceutically acceptable carrier.

31. A method for the treatment or prevention of diseases or disorders where bromodomain inhibition is desired comprising administering to a subject in need thereof a therapeutically effective amount of a compound according to claim 1.

32. The method according to claim 31, wherein the disease or disorder is an autoimmune disease, an inflammatory disease, or cancer.

33-34. (canceled)
